(19) United States
(12) Patent Application Publication
(10) Pub. No.: US 2025/0333744 A1
(43) Pub. Date: Oct. 30, 2025

(54) Polynucleotide Treatments for Charcot-Marie-Tooth Disease

(71) Applicants: Shift Pharmaceuticals Holding Inc., Columbia, MO (US); The Curators of the University of Missouri, Columbia, MO (US)

(72) Inventors: Stephen D. O'Connor, Columbia, MO (US); Christian Lorson, Columbia, MO (US)

(21) Appl. No.: 18/855,533

(22) PCT Filed: Apr. 13, 2023
(86) PCT No.: PCT/US2023/065728

§ 371 Date: Oct. 9, 2024

Related U.S. Application Data
(60) Provisional application No. 63/331,044, filed on Apr. 14, 2022.
(60) Provisional application No. 63/331,045, filed on Apr. 14, 2022.

(51) Int. Cl. C12N 15/113; A61P 21/00; A61P 25/02

(52) U.S. Cl. CPC C12N 15/1138; A61P 21/00; A61P 25/02; C12N 2310/11; C12N 2310/314; C12N 2310/3233; C12N 2320/33

(57) ABSTRACT

Inherited peripheral neuropathies, also known as Charcot-Marie-Tooth disease (CMT), are one of the most common heritable diseases of the nervous system, affecting approximately 1 in 2,500 individuals. This disclosure is directed to therapeutic strategies for the treatment of Charcot-MarieTooth disease (CMT) via targeting PMP22 pre-mRNA with antisense oligonucleotides (ASOs), including methods and compositions for the same.

### REFERENCE TO SEQUENCE LISTING SUBMITTED ELECTRONICALLY

This international application claims the benefit of U.S. Provisional Application 63/331,044, filed on Apr. 14, 2022 and U.S. Provisional Application 63/331,045, filed on Apr. 14, 2022, both of which are incorporated herein in their entireties.

### REFERENCE TO SEQUENCE LISTING SUBMITTED ELECTRONICALLY

The contents of the electronically submitted sequence listing in ST26 format (Name UMC_226824.xml; Size: 242,797 bytes; and Date of Creation: Apr. 13, 2023) filed with this application is incorporated herein by reference in its entirety.

### BACKGROUND

This disclosure is directed to therapeutic strategies for the treatment of Charcot-Marie-Tooth disease (CMT) via targeting PMP22 mRNA with antisense oligonucleotides (ASOs), including methods and compositions for the same.

Inherited peripheral neuropathies, also known as Charcot-Marie-Tooth disease (CMT), are one of the most common heritable diseases of the nervous system, affecting approximately 1 in 2,500 individuals (Krajewski et al., 2000; Braathen, 2012). CMT1A, the most common form of CMT, is a demyelinating neuropathy caused by genetic duplication of the peripheral myelin protein-22 (PMP-22) gene (SEQ ID NO: 1) (Patel et al., 1992; Timmerman et al., 1992; Valentijn et al., 1992; Matsunami et al., 1992). As the name suggests, PMP22 is an essential structural component of the myelin sheath that surrounds axons (Lee et al., 2014; Mittendorf et al., 2017; Snipes et al., 1992). Functionally, myelin acts as a biological insulator that facilitates the efficient transmission of electrical impulses along an axon. While myelin is produced in the CNS by glial cells, Schwann cells (SC) are responsible for the production of PMP22 and myelin in the peripheral nervous system (PNS). While too much PMP22 results in the development of CMT1A, an autosomal dominant disease, it is equally important to maintain sufficient PMP22 expression as the loss of PMP22 results in a distinct neuropathy called hereditary neuropathy with predisposition to pressure palsy (HNPP). Taken together, PMP22 is an essential component of the myelin sheath and the delicate homeostatic balance of this gene should be paramount in the development of effective therapeutics.

Mutations in more than 90 distinct genes cause CMT, the most common of which is a 1.4-Mb duplication on human chromosome 17, classified as CMT1A. The peripheral myelin protein 22 (PMP22) gene, which encodes the major myelin protein, peripheral myelin protein 22, resides within the 1.4-Mb duplicated interval. PMP22 is an intrinsic membrane protein of myelin that alters lipid organization/distribution and is developmentally induced within Schwann cells as they initiate myelination of peripheral nerves.

Studies in rodents have demonstrated that overexpression of PMP22 is sufficient to cause a demyelinating neuropathy (Magyar et al., 1996; Sereda et al., 1996; Huxley et al., 1996), and proof-of-concept conditional knockout studies demonstrated that reduction of PMP22 overexpression led to remyelination (Perea et al., 2001). Interestingly, the deletion of the same 1.4-Mb region results in the loss of a PMP22 allele and causes a distinct neuropathy known as hereditary neuropathy with liability to pressure palsies (Chance et al., 1993), further demonstrating that gene dosage is the critical determinant in these neuropathies. Finally, elevated levels of PMP22 protein have been demonstrated in dermal or sural nerves of CMT1A patients.

Several approaches to reduce PMP22 expression have been proposed, yet no therapy is currently available to patients. For instance, PMP22 overexpression in rodent models can be reduced by high-dose ascorbic acid (Cortese et al., 2020); however, in clinical trials, ascorbic acid did not reduce the level of PMP22 mRNA in skin biopsies from treated CMT 1A patients (Eichinger et al., 2018; Gautier et al., 2021; Kagiava et al., 2018). Progesterone antagonists and GABAB agonists have also been shown to reduce PMP22 mRNA expression (Lee et al., 2020; Massade and Charbel, 2020), but their potential is hampered by diverse effects on the gene-regulation program of Schwann cells and possibly other cell types, which may complicate a chronic treatment of an inherited disease.

CMT1A is monogenic; the disease gene has been identified; and inhibition of PMP22 expression can be accomplished through a variety a molecular mechanisms. Previously, a panel of 2′-O-2-methoxyethyl phosphorothioate based backbone (2′ MOE) ASOs were developed and analyzed as a means to interfere with PMP22 expression (Zhao et al., 2018). In this report by Zhao et al., ASOs were identified that decreased PMP22 expression in several important cellular and in vivo contexts, including K-562 cells and the C22 transgenic mouse model. Importantly, each of these experimental contexts is predicated upon the presence of the human PMP22 gene. In C22 mice, ASO treatment decreased PMP22 expression and significantly improved the CMT phenotype, including neuronal pathology, degree of myelination, and CMAP/MNCV.

Other approaches for altering PMP22 RNA translation have been proposed to knock down the entire process by developing molecules to interfere with the 3′ UTR of the human PMP22 gene (e.g., world wide web at www.jci.org/articles/view/96499; U.S. Pat. No. 11,136,577). In patent application WO2020/132558 A1, the use of gapmers is described to enhance the hybridize to a target piece of RNA and silence the gene through the induction of RNase H cleavage.

These approaches have one aspect in common, namely they entirely eliminate the full length PMP22 RNA expression or destroy the RNA during some stage of the genomic cycle. While this approach can be effective, overdosing of patients with too much drug may lead to secondary diseases and symptoms as described above if too much PMP22 is eliminated.

Thus, there remains a need to develop effective treatments for CMT disease.

### SUMMARY

This disclosure relates to molecules (small molecules, antisense oligonucleotides, antibodies, etc) that bind to the pre-mRNA region of human PMP22 related to exon splicing. The molecules bind to one (or more) of these key regions of the pre-mRNA prior to splicing to induce an exon skipping event during translation/transcription that results in mRNA being produced that is similar to the full-length version of the natural mRNA, but missing one or more exons or a portion thereof. The resulting exon-skipped mRNA is stable and measurable, either in vitro, in vivo, or in situ.

Provided for herein is a composition comprising an antisense oligonucleotide (ASO) that comprises or consists of a complementary region that is complementary, or complementary except for one, two, three, four, or five mismatched nucleotides, to a target region of a PMP22 pre-mRNA. In certain embodiments, binding in a cell of the complementary region of the ASO to the target region of the PMP22 pre-mRNA induces exon skipping during RNA transcription. In certain embodiments, the induced exon skipping reduces full-length PMP22 mRNA production. In certain embodiments, the induced exon skipping produces an exon-skipped PMP22 mRNA.

Also provided for herein is a method of decreasing the amount of full-length PMP22 mRNA expression in a cell. Such method comprises administering to the cell an exon skipping inducing composition comprising an antisense oligonucleotide (ASO) of this disclosure. In certain embodiments, an PMP22 exon-skipped mRNA is produced. In certain embodiments, the amount of PMP22 protein produced in the cell is decreased.

Also provided for herein is a method of producing an exon-skipped PMP22 pre-mRNA. Such method comprises administering to a cell an exon-skipping inducing composition comprising an antisense oligonucleotide (ASO) of any this disclosure. In certain embodiments, the amount of full-length PMP22 mRNA expression in the cell is decreased. In certain embodiments, the amount of functional PMP22 protein produced in the cell is decreased.

Also provided for herein is a method of treating Charcot-Marie-Tooth disease. Such method comprises administering to a subject in need thereof an exon-skipping inducing composition comprising an antisense oligonucleotide (ASO) of any this disclosure.

Also provided for herein is a composition comprising an antisense oligonucleotide (ASO) comprising or consisting of a complementary region that is complementary, or complementary except for one, two, three, four, or five mismatched nucleotides, to a target region of a PMP22 pre-mRNA. In certain embodiments, the target region of the PMP22 pre-mRNA comprises an intron/exon junction of one of the coding exons.

### BRIEF DESCRIPTION OF THE DRAWINGS/FIGURES

FIG. 1. FIG. 1 shows PMP22 pre-mRNA (top), resulting in full length mRNA that contains all of the amino acid coding Exons (middle) and functioning protein (bottom). Introns between each exon are shown as double horizontal lines and the base pair sequences are removed during splicing.

FIG. 2. FIG. 2 shows the PMP22 gene process when an anti-sense oligonucleotide (ASO) is added that hybridizes to a portion of the premRNA and induces a skipping event when the pre-mRNA is converted to mRNA. When an exon skipping anti-sense oligonucleotide is present in the cells during RNA transcription, a skipped exon product is formed which will lead to a non-functioning protein.

FIG. 3A,B,C. FIG. 3 is a schematic of the changes in structure to a pre-mRNA during splicing.

FIG. 4A-D. FIG. 4 shows a schematic of a number of strategies for biding to the pre-mRNA to induce exon skipping.

FIG. 5A,B. FIG. 5 shows the sequence of PMP22 gene around the 5′- and 3′-regions of Exon 3 (all caps) and associated upstream and downstream introns. 5A shows the 5′-end of the exon and 5B shows the 3′-end. A number of different representative 25-mer oligonucleotides ASO sequences of the present disclosure are shown (below the sequence at the top) that may produce a skipping of Exon 3 during conversion from pre-mRNA to mRNA. Various methods of synthesizing ASO are available (both solid phase and solution phases and, e.g., with 5′ to 3′ directionality or with 3′ to 5′ directionality) to one of ordinary skill in the art as long as the final ASO has the sequence disclosed (or including any mismatches as provided for herein) and hybridizes to the target region or a portion of the target region. Six representative morpholino anti-sense oligo sequences that were designed (and synthesized) to bind to the intro/exon junctions are shown where data is presented. 5A: SEQ ID NOs: 2-34; SHC-006 25-mer (SEQ ID NO: 71); SHC-001 24-mer (SEQ ID NO: 72); SHC-005 25-mer (SEQ ID NO: 73). 5B: SEQ ID NOs: 35-70; SCH-010 21-mer (SEQ ID NO: 74); SCH-012 20-mer (SEQ ID NO: 75).

FIG. 6. FIG. 6 shows the results from PCR amplification followed by gel electrophoresis analysis of the selected Exon 3 skipping ASOs from FIG. 5. A scramble injection control (that does not effect PMP22 pre-mRNA or mRNA) is shown as well.

FIG. 7. FIG. 7 shows RT-PCR from C3 mice (liver) treated with SHC-012 (SEQ ID NO: 75), a scramble (2×) ASO, or a water control. Human full-length PMP22 mRNA is detectable in addition to the GAPDH control. SHC-012-induced exon 3 skipping (confirmed by sequence) is present as marked.

FIG. 8. FIG. 8 shows the PCR results (and corresponding change in amount of full-length PMP22 mRNA) for a number of tissues after a single subcutaneous injection of SHC-012 (SEQ ID NO: 75) was given and the animals sacrificed 2 days later. The data in FIG. 8 represents percent remaining. Specifically, for each tissue type we quantitated the total amount of PMP22 full-length mRNA per tissue type and compared to the SHC-012 animals. 20% remaining indicates the 80% of the pre-mRNA was blocked from making full-length mRNA and made exon-skipped mRNA instead.

FIG. 9. FIG. 9 shows the amount of time taken to walk across the dowel apparatus described for different treatment groups (using SHC-012; SEQ ID NO: 75), scramble animals, and wild-type mice. For all groups, 3 animals were in each group and the histogram shows the average values and standard deviations. P values were calculated (to determine confidence levels against the scramble control) for each treatment group. For each group, p<0.05. All data is for average values of all animals that are 12 weeks old.

FIG. 10. FIG. 10 shows the amount of time spent on a rotarod apparatus for the scramble, wild-type, and treatment groups first injected at 5-weeks of age. For all groups, 3 animals were in each group and the histogram shows the average values and standard deviations. P values were calculated (to determine confidence levels against the scramble control) for each treatment group and are shown. All data is for average values of all animals that are 12 weeks old.

FIG. 11A,B. FIG. 11 shows the sequence of PMP22 gene around the 5′- and 3′-regions of Exon 4 (all caps) and associated upstream and downstream introns. 11A is the 5′-end of the exon and 11B is the 3′-end. A number of different 25-mer oligonucleotides ASO sequences of this disclosure are shown (below the sequence at the top) that may produce a skipping of Exon 4 during conversion from pre-mRNA to mRNA. Six representative morpholino anti-sense oligo sequences that were designed (and synthesized) to bind to the intro/exon junctions are shown where data is presented. 11A: SEQ ID NOs: 76-110; SHC-029 21-mer (SEQ ID NO: 146); SHC-028 20-mer (SEQ ID NO: 147); SHC-027 20-mer (SEQ ID NO: 148). 11B: SEQ ID NOs: 111-145; SCH-031 21-mer (SEQ ID NO: 149); SCH-030 20-mer (SEQ ID NO: 150); SCH-032 20-mer (SEQ ID NO: 151).

FIG. 12. FIG. 12 shows the results from PCR amplification followed by gel electrophoresis analysis of the selected Exon 4 skipping ASOs from FIG. 11.

FIG. 13. FIG. 13 shows quantitation results using two different methodologies from the gel results for 3 of the Exon 4 skipping compounds.

FIG. 14. FIG. 14 shows the 5′- and 3′-ends of Exon 3 (top) and 3 different ASO compounds that were designed and tested that bridge the target regions (shown for each). These ASO target regions of the Exon that are non-continuous and will have a different effect on the three dimensional structure of the pre-mRNA than those described above. SEQ ID NOs: 152-162 and SEQ ID NOs: 233-238; SHC-043 (SEQ ID NO: 156); SHC-044 (SEQ ID NO: 159); SHC-045 (SEQ ID NO: 162); SHC-046 (SEQ ID NO: 235); SHC-047 (SEQ ID NO: 238).

FIG. 15. FIG. 15 shows the results from PCR amplification followed by gel electrophoresis analysis of the selected Exon 3 skipping ASOs from FIG. 14.

FIG. 16A,B. FIG. 16 shows the sequence of PMP22 gene around the 5′- and 3′-regions of Exon 2 (all caps) and associated upstream and downstream introns. 15A is the 5′-end of the exon and 15B is the 3′-end. 16A: SEQ ID NOs: 163-197. 16B: SEQ ID NOs: 198-232.

FIG. 17. FIG. 17 shows images of the sciatic nerve for WT, untreated, and treated animals (top) and images from the peroneal portion of the nerve (bottom).

FIG. 18. FIG. 18 shows a TEM image at higher magnification of portions of a Peroneal nerve.

FIG. 19. FIG. 19 shows electrophysiology plots from the sciatic gastric section of sedated mice (top). FIG. 19 (bottom) shows the average values of MUNE and CMAP from 3 measured animals per group.

FIG. 20. FIG. 20 shows results for treatment groups (with the scramble animals' group and wild-type animals shown for comparison at 4 months of age). Each data set of the histogram is an average of 4 days dowel travers time at the end of each month.

FIG. 21. FIG. 21 shows the results of the dowel traverse time experiment (with 3 month old wild-type animals also plotted for comparison).

### DETAILED DESCRIPTION

### Definitions

To the extent necessary to provide descriptive support, the subject matter and/or text of the appended claims is incorporated herein by reference in their entirety.

It will be understood by all readers of this written description that the exemplary embodiments described and claimed herein may be suitably practiced in the absence of any recited feature, element or step that is, or is not, specifically disclosed herein.

Unless defined otherwise, technical and scientific terms used herein have the same meaning as commonly understood by one of ordinary skill in the art to which this disclosure is related.

It is to be noted that the term “a” or “an” entity refers to one or more of that entity; for example, “a compound,” is understood to represent one or more compounds. As such, the terms “a” (or “an”), “one or more,” and “at least one” can be used interchangeably herein.

Furthermore, “and/or” where used herein is to be taken as specific disclosure of each of the specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Numeric ranges are inclusive of the numbers defining the range. Even when not explicitly identified by “and any range in between,” or the like, where a list of values is recited, e.g., 1, 2, 3, or 4, the disclosure specifically includes any range in between the values, e.g., 1 to 3, 1 to 4, 2 to 4, etc.

The headings provided herein are solely for ease of reference and are not limitations of the various aspects or aspects of the disclosure, which can be had by reference to the specification as a whole. Accordingly, the terms defined immediately below are more fully defined by reference to the specification in its entirety.

As used herein, the term “identity,” e.g., “percent identity” to an amino acid sequence or to a nucleotide sequence disclosed herein refers to a relationship between two or more nucleotide sequences or between two or more amino acid sequences. When a position in one sequence is occupied by the same nucleic acid base or amino acid in the corresponding position of the comparator sequence, the sequences are said to be “identical” at that position. The percentage “sequence identity” is calculated by determining the number of positions at which the identical nucleic acid base or amino acid occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. In order to optimally align sequences for comparison, the portion of a nucleotide or amino acid sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using, e.g., the program “BLAST” which is available from the National Center for Biotechnology Information, and which program incorporates the programs BLASTN (for nucleotide sequence comparison) and BLASTP (for amino acid sequence comparison), which programs are based on the algorithm of Karlin and Altschul (Proc. Natl. Acad. Sci. USA 90(12):5873-5877, 1993).

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology, but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

By an “isolated” polypeptide or a fragment, variant, or derivative thereof or the like is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are recombinant polypeptides that have been separated, fractionated, or partially or substantially purified by any suitable technique.

The term “polynucleotide” is intended to encompass a singular nucleic acid as well as plural nucleic acids, and refers to an isolated nucleic acid molecule or construct, e.g., pre-messenger RNA (pre-mRNA), messenger RNA (mRNA), or plasmid DNA (pDNA). A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “coding region” is a portion of nucleic acid comprising codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode a selection marker gene and a gene of interest. In addition, a vector, polynucleotide, or nucleic acid can encode heterologous coding regions, either fused or unfused to a nucleic acid encoding a polypeptide subunit or fusion protein as provided herein. Heterologous coding regions include without limitation specialized elements or motifs, such as a secretory signal peptide or a heterologous functional domain.

As used herein, an “exon” refers to the portion of a DNA or RNA sequence that results in the synthesis of an amino acid sequence.

As used herein, an “intron” refers to the portion of a DNA or RNA sequence that does not result in the synthesis of an amino acid sequence.

In certain aspects, the polynucleotide or nucleic acid is DNA. In the case of DNA, a polynucleotide comprising a nucleic acid that encodes a polypeptide normally can include a promoter and/or other transcription or translation regulatory elements operably associated with one or more coding regions. An operable association or linkage can be when a coding region for a gene product, e.g., a polypeptide, can be associated with one or more regulatory sequences in such a way as to place expression of the gene product under the influence or control of the regulatory sequence(s). Two DNA fragments (such as a polypeptide coding region and a promoter associated therewith) can be “operably associated” or “operably linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA only in predetermined cells. Other transcription regulatory elements, besides a promoter, for example enhancers, operators, repressors, and transcription termination signals, can be operably associated with the polynucleotide to direct cell-specific transcription.

A variety of transcription regulatory regions are known to those skilled in the art. These include, without limitation, transcription regulatory regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription regulatory regions include those derived from vertebrate genes such as actin, heat shock protein, bovine growth hormone and rabbit beta-globin, as well as other sequences capable of controlling gene expression in eukaryotic cells. Additional suitable transcription regulatory regions include tissue-specific promoters and enhancers.

Similarly, a variety of translation regulatory elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other aspects, a polynucleotide can be RNA, for example, in the form of a pre-mRNA or messenger RNA (mRNA).

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain aspects, the native signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse 8-glucuronidase.

A “vector” is nucleic acid molecule as introduced into a host cell, thereby producing a transformed host cell. A vector can include nucleic acid sequences that permit it to replicate in a host cell, such as an origin of replication. A vector can also include one or more selectable marker gene and other genetic elements known in the art. Illustrative types of vectors include plasmids, phages, viruses and retroviruses.

A “transformed” cell, or a “host” cell, is a cell into which a nucleic acid molecule has been introduced by molecular biology techniques. As used herein, the term transformation encompasses those techniques by which a nucleic acid molecule can be introduced into such a cell, including transfection with viral vectors, transformation with plasmid vectors, and introduction of naked DNA by electroporation, lipofection, and particle gun acceleration. A transformed cell or a host cell can be a bacterial cell or a eukaryotic cell.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into pre-mRNA and messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

The term “pharmaceutical composition” refers to a preparation or mixture of substances suitable for administering to a subject, i.e., that is in such form as to permit the biological activity of the active ingredient to be effective, and that contains no additional components that are unacceptably toxic to a subject to which the composition would be administered. Such composition can be sterile. For example, a pharmaceutical composition may comprise an oligomeric compound and a sterile aqueous solution.

As used herein, “pharmaceutically acceptable carriers or diluents” are suitable for administration. Certain such carriers enable pharmaceutical compositions to be formulated as, for example, tablets, pills, dragees, capsules, liquids, gels, syrups, slurries, suspension, and lozenges for the oral ingestion by a subject. In certain embodiments, a pharmaceutically acceptable carrier or diluent is sterile water, sterile saline, sterile buffer solution, or sterile artificial cerebrospinal fluid.

As used herein “pharmaceutically acceptable salts” are physiologically and pharmaceutically acceptable salts of compounds. Pharmaceutically acceptable salts retain the desired biological activity of the parent compound and do not impart undesired toxicological effects thereto.

As used herein, an “antisense compound” is a compound capable of achieving at least one antisense activity. In certain embodiments, an antisense compound comprises an antisense oligonucleotide (ASO) and optionally one or more additional features, such as a conjugate group or terminal group. In certain embodiment, an antisense compound has been engineered and synthesized to contain non-naturally occurring backbone structures (such as changes in the sugars and/or phosphate backbone). In certain embodiments, the antisense compounds have a morpholino backbone.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

For purposes of this disclosure, the entire human PMP22 gene was downloaded from the University of California Santa Cruz genome.ucsc.edu database. Consistent with standard nomenclature, as shown in the Figures, regions of sequence in introns or non-coding portions of the genome appear in lower case letters. Regions of sequence encoding amino acids appear in upper case letters. For the database used and corresponding sequences shown in the Figures, the UCSC database above was used and the option to download the sequences from the Human Assembly December 2013 (GRCh38/hg38) with the protein coding option was selected. If other databases are available and acceptable or become available and acceptable with slight sequence variations, one skilled in the art would understand this description to also cover those variants.

As used herein, the term “complementary” in reference to an oligonucleotide refers to two nucleic acid singles strands or portions of a single strand capable of hybridizing into a double-stranded sequence via hydrogen bonding of complementary bases. Complementary bases include adenine (A) and thymine (T), adenine (A) and uracil (U), cytosine (C) and guanine (G), 5-methyl cytosine (mC) and guanine (G). Complementary oligonucleotides and/or nucleic acids do not need to be complementarity at each positions. Some mismatches can be tolerated. As used herein, “fully complementary” or “100% complementary” in reference to oligonucleotides means that the two oligo-strands have complementary bases at each corresponding position. In certain embodiments, complementary oligonucleotides have only at least 70% complementary bases at each corresponding position.

As used herein, “hybridization,” hybridizing, and the like means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleotides.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “exon skipping” can involve skipping an entire exon or a portion of an exon.

As used herein, an exon-skipping compound” relates to any compound that binds to a pre-mRNA species and induces transcribed mRNA that is stable and measurable, but not full-length (i.e., exon-skipped mRNA).

While a gene or genetic locus may be identified by a particular reference sequence, e.g., the human PMP22 gene (SEQ ID NO: 1), it is understood that the gene corresponding to the reference sequence can comprise various allelic forms or variations in sequence and still be considered by one of ordinary skill in the art to be the same gene.

It is understood that the nucleic acid sequences set forth herein and in each corresponding sequence ID Number (SEQ ID NO) are independent of any modification to a sugar moiety, an inter-nucleoside linkage, or a nucleobase. As such, any nucleic acid of this disclosure, including oligonucleotides, may comprise, independently, one or more modifications to a sugar moiety, an inter-nucleoside linkage, or a nucleobase.

### Overview

Provided herein is a direct therapeutic strategy targeting PMP22 RNA with antisense oligonucleotides (ASOs) comprising a method of using ASOs that bind to and interact with the pre-mRNA of the PMP22 gene cascade and effectively “splice out” certain portions of the mRNA by binding to junctions between the spacer region in the pre-mRNA and the exon regions that convert into full mRNA. In certain embodiments, the ASOs do not prevent the rest of the PMP22 gene exons from being expressed into amino acids. With one or more of the exons removed or partially removed from the mRNA, the resulting altered amino acid sequence is shorter or different than the naturally occurring PMP22 amino acid sequence, causing loss of function of the resulting protein and/or enhanced degradation compared to the naturally occurring protein (i.e., a reduction/decrease of functional PMP22 protein). In other embodiments, the splicing event causes the resulting alternatively spliced mRNA (exon-skipped mRNA) to be degraded within the cell at a faster rate than the full-length mRNA, thus lowering protein production.

For embodiments where a exon-skipped mRNA is produced using an ASO of this disclosure, it is possible to monitor the ratio of the full-length mRNA to exon-skipped mRNA over time, both in the target tissues involved and in the corresponding blood levels. In this manner, the molecular activity of a drug can be specifically measured in subjects (e.g., laboratory animals and/or patients including human patients over time). The amount of exon-skipped mRNA can be correlated to various downstream effects of the drug that can have positive outcomes on patients, such as to lower the effect of functional protein levels and the phenotypic response of the patient or laboratory animal to the drug. In this manner, embodiments of the present disclosure can be used as a diagnostic method to confirm the activity of a drug in a specific subject, alter the amount of the drug given to a patient to achieve a desirable response, and/or alter the dosing frequency of the drug to achieve a desirable response, etc.

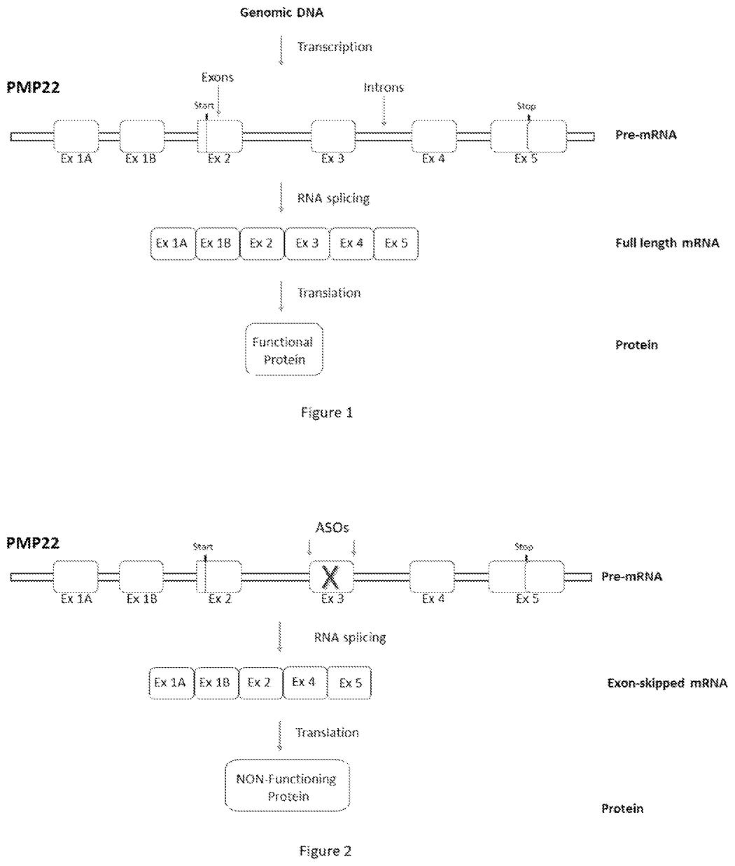

Normal full-length mRNA production occurs in cells and leads to fully functionable PMP22 protein (FIG. 1). In FIG. 1, the Exons (2-5) within the marked start regions contain the nucleic acid base pairs that code for the amino acids that make up function, full-length PMP22 protein. The top portion of FIG. 1 shows pre-mRNA that contains the exon regions as well as the intron regions. The introns are spliced out of the pre-mRNA to produce mRNA shown below the pre-mRNA. Translation then occurs producing proteins form Exons 2-5 between the start and stop portions as noted. CMT1A patients have 2 copies of the PMP22 gene from birth, which leads to overproduction of full-length PMP22 mRNA and full-length PMP22 protein and is a major contributing factor in disease development.

Previously described approaches attempt to alter PMP22 protein levels by “knocking down” or “silencing” mRNA. Some methods have described the addition of drug-like molecules that either act with regulatory portion of the pre-mRNA or mRNA to stop all production. Other methods have proposed a gap-mer like strategy to enhance mRNA cleavage through RNase H, destroying the mRNA to lower PMP22 production. These methods have a distinct disadvantage in their molecular effectiveness can only be monitored by measuring the total amount of full-length mRNA or protein against control animals or patients to monitor activity. Since the presence of at least a certain amount of PMP22 protein being produced is required for normal physical well-being, removing too much PMP22 protein can result in numerous problematic conditions including but not limited to a distinct neuropathy known as hereditary neuropathy with liability to pressure palsies, loss of peripheral never functioning, and worsening of physical symptoms, or even death. Thus, to achieve a satisfactory clinical outcome, it is critical to remove a significant amount of PMP22 protein without removing all of the protein present in a patient's cells. This exact goal is unlikely to be unachievable with a single dose schedule for all patients and all lifestyles, since numerous factors, including age, weight, physical lifestyle, natural history, stage of disease progression, etc., will affect the total PMP22 protein production and stability over time in different patients. Since there is huge natural variation in mRNA levels and protein levels in patients due to a variety of physical or behavioral factors, monitoring the molecular activity using total mRNA reduction approaches on a patient-by-patient basis is simply not possible.

One embodiment of this disclosure is shown in FIG. 2 and relates to any compounds that interact with the pre-mRNA shown in FIG. 2 to eliminate one (or more) of the exons during translation from pre-mRNA to mRNA and allow the other exons to be included, creating a stable, measurable new exon-skipped mRNA that is similar to full-length mRNA but does not code for fully functioning PMP22 protein. FIG. 2 schematically shows a compound that would eliminate Exon 3, but other examples are described below for Exon 2 and Exon 4.

The “exon-skipping” event during splicing could result in either a completely concatenated non-functioning protein or out-of-frame translation.

Thus, disclosed herein is a method where the amount of full-length PMP22 mRNA, which will lead to effective protein, and the presence of exon-skipped mRNA, that will lead to non-function protein, can be compared within a given subject, making this monitoring possible with the practical natural variation of background mRNA and protein levels. In certain embodiments, this can be done in animals studies, both simplifying these studies and providing more information within a given study. And, in certain embodiments, this can be done in human patients receiving treatment.

This approach varies from other previous drug approaches to genetic disorders in which it is either simply not possible to monitor or not monitorable in a practical senses since no internal control within the patient sample is present. Thus, factors such as sample collection, patient variance, variance in analytical methods, etc., make these studies impossible or not practical. In contrast, the approach of this disclosure leads to two measurable markers within the patient sample and the ratio of those two markers is a defining factor of the drugs activity. In certain embodiments, the amount of exon-skipped mRNA in an animal or patient sample is used to track therapeutic activity. In another embodiment, the amount of exon-skipped mRNA is compared to the amount of full-length mRNA and that ratio is used to track the therapeutic activity.

For CMT1A, it is desirable to target PMP22 protein production. Further, it would be highly desirable to do so by altering the patient's dosing schedule. Dosing requirements may be highly dependent on a number of patient factors including weight, age, disease progression, lifestyle, etc. Previously described approaches for treating CMT1A (and other monogenic and multi-genetic) diseases do not allow an accurate methodology for tracking the mRNA production and thus total (or partial) protein production from that mRNA.

For most classical drug development, the overall strategy is to study drug effects versus concentration delivered over a large number of cellular and animal experiments to correlate the drugs effect at altering phenotypic responses. Drugs can then be studies in humans (typically at multiple dosing strategies mainly focuses on safety and toxicity profiling) in order to come up with a single recommended dose across a target patient population. This often leads to drugs that show a modest effect in clinical setting, but often leads to showing no measurable phenotypic effect whatsoever in a subset of the patient population. Often, these unresponsive patients are simply being dosed ineffectively (either not enough or too much of the drug) based on semi-correlated previous studies.

When ASOs of this disclosure are added to cells (or subject, e.g., animals or humans for in vivo treatment), a portion of the ASOs interact with the pre-mRNA, forcing that exon (or more exons) to be skipped during transcription. An mRNA with the “skipped” exon (“exon-skipped mRNA”) is still formed but produces non-functioning protein. One important advantage of this approach is that the exon-skipped mRNA molecule can be quantitated as a measurement of drug activity at a molecular level and therefore is not dependent on certain other environmental and experimental parameters (that are always present) which can confound the results obtained

Provided for herein is a new class of therapeutics that elicit a molecular response in subjects wherein both the native unaffected, full-length mRNA and the exon-skipped mRNA can be monitored during the course of treatment and related to downstream protein production. In certain embodiments, samples can be taken from target tissues of interest. In certain embodiments, samples can be taken from other (more accessible) portions of the body, such as blood plasma. This approach allows monitoring of the effect of the therapeutic compositions of this disclosure, both during animals studies and in actual patients during clinical deployment.

While PCR is an elegantly sensitive and specific method for analyzing the amount of RNA present—for example in both the target tissues of interest and in corresponding plasma levels—since RNA is not stable and the amount of RNA present in the blood may be significantly lower than the amount and nature of the RNA in the tissue, PCR monitoring can become problematic. Monitoring protein production in the target tissue and corresponding plasma levels can be even more problematic, since the protein levels may be even more variable based on environmental factors including patient activity and inherent protein stability differences in the target tissues versus other portion of the body during biodistribution.

An important aspect of this disclosure is the unique approach of targeting the junction between the introns and exons of the pre-mRNA prior to transcription. The junction points between introns and exons are highly susceptible for ASO targeting (compared to the non-overlapping regions of only intro or only exon) for a number of reasons. First, the binding of the ASO to the target it highly dependent on the 3-dimensional availability of the RNA to receive the ASO. Oligonucleotides in cells have complex 3D structures and this unique position will “open” the structure and make the binding site of our proposed drugs available. Second, the binding of any ASO to a genetic target will be in “competition” with the proteins and enzymes (and other molecules) for those binding sites and can be displaced by said proteins. Even if the ASO compounds binds to the target, if it displaced by the functioning enzyme (during transcription, translation, etc.) it will effectively not function effectively. Surprisingly, it has been discovered that these junction points are much more available for active binding and competitive strength (to stay bound) as compared to non-junction sites.

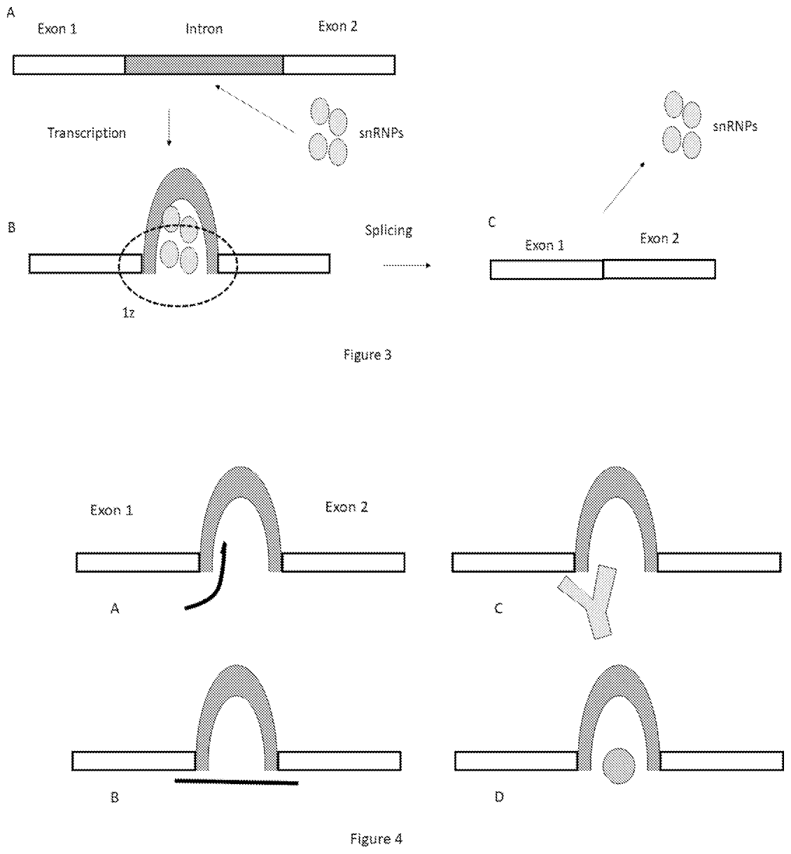

FIG. 3 and FIG. 4 describe an overall targeting approach. FIG. 3A shows a schematic of Exon 1, the intron region, then Exon 2 (referred to as the pre-mRNA). Prior to the elimination of introns to join to sequential exons to one another during the conversion of pre-mRNA to mRNA, the pre-mRNA forms a three dimensional structure in the presence of snRNPs to create an intermediate structure shown in FIG. 3B where the 3′-end of one Exon1 is brought into close proximity to the 5′-end of Exon 2. Once this complex 3D structure is created, biological molecules (such as enzyme) can act on the structure (in the presence of snRNPs and other cofactors) to remove the intron region of the pre-mRNA and chemically join two adjacent exons (this is shown in FIG. 3 as Exon 1 and Exon 2, however other consecutive exons within a gene can are joined in a similar manner). This disclosure relates to binding molecules to selective regions within this genome to disrupt a specific exon-intron-exon junction and allow the remainder of the transcription to occur (either in part or whole). This region is shown (lz, dotted circle in FIG. 3B) and the 3D structure is critical in this region in order for the splicing process to occur efficiently. This region is critical due to a number of factors including the enzymes recognizing the 3D structure, the chemical nature of the 3D structure, the close proximity of the ends of the two exons which will be joined, etc. Once splicing occurs, the two ends of Exon 1 and 2 are joined (FIG. 3C) forming the mRNA.

In certain embodiments an anti-sense oligonucleotide (ASO) is introduced that specifically binds or hybridizes to a region of the intron and/or exon itself that is the target of the exon skipping. In such embodiments, the ASO added comprises a sequence that is complimentary to the pre-mRNA in a region close to the proximal site described (l z in FIG. 3) in order to disrupt exon inclusion and produce a final mRNA product that does not include the exon targeted but does include other exons within the gene targeted where no drug has bound. The presence of the ASO within the nucleus of the cell (where RNA translation occurs) will form an equilibrium with the pre-mRNA that will be directly affected by the amount of the drug present, the amount of pre-mRNA present, binding efficiency to the region targeted, strength of the attachment once bound, etc. In certain embodiments of this disclosure, an amount of drug is introduced that does not force exon skipping of all the pre-mRNA present in the cell. Thus, in any given tissue sample, both full-length PMP22 mRNA and exon-skipped mRNA will be produced and the amount of skipping that occurs will be dependent on factors such as the chemical composition of the drug and the amount of drug added. Other factors may affect the ratio of the exon skipping. Specifically, certain embodiments of the disclosure relate to molecules that do not completely eliminate all full-length mRNA production. ASOs that are complimentary to continuous regions of the pre-mRNA can target a 3′-end of an exon, a 5′-end of an exon, or other regions of the intron or exon, provided that they induce a measurable exon skipping event where shortened, non-full length exon-skipped mRNA is produced.

The length of the ASO shown in FIG. 4B can vary depending on the desired effect. In this disclosure, ASOs are disclosed that specifically target the PMP22 pre-mRNA, thus a minimum length of ASO bases is preferable that is specific to the PMP22 mRNA but does not also specifically bind to other areas of the human genome. Additionally, very long ASOs can be problematic in their use, since long strands of DNA, RNA, etc. may fold onto themselves and not be available for binding to the target pre-mRNA. Manufacturing concerns also come under consideration with very long ASOs. Thus, in certain embodiments, an ASO is between any of about 8, 9, 10, 11, 12, 13, 14, 15, 16, 17, 18, 19, 20, 21, 22, 23, 24, 25, 26, 27, 28, or 29 and any of about 9, 10, 11, 12, 13, 14, 15, 16, 17, 18, 19, 20, 21, 22, 23, 24, 25, 26, 27, 28, 29, or 30 nucleotides long. In certain embodiments, an ASO is between about 12 and 30 nucleotides long. In certain embodiments, an ASO is between about 15 and 25 nucleotides long. In certain embodiments, an ASO is between about 18 and 25 nucleotides long.

The ASOs of this disclosure do not necessarily have to perfectly match (i.e., be 100% complementary to) all of the target pre-mRNA bases for hybridization. For example, in certain embodiments an 18-mer ASO maybe designed and synthesized where one or one or more of the bases is not complimentary to the target pre-mRNA, but will still hybridize/possess antisense activity. For example in certain embodiments, two or two or more of the ASO/target region base pairs can be considered “mis-matches” where they are not complimentary. In certain embodiments, an ASO comprises a complementary region that is complementary, or complementary except for one, two, three, four, or five mismatched nucleotides, to a target region of the pre-mRNA. In certain embodiments, an ASO comprises a complementary region that is at least 70%, 75%, 80%, 85%, 90%, 95%, 98%, or 99% complementary to a target region of the pre-mRNA.

Another embodiment of this disclosure is shown in FIG. 4B, where an ASO is used that does not bind to a continuous portion of the pre-mRNA, but rather spans two different portions of the pre-mRNA. For example, in FIG. 4B, the ASO shown can have bases that are complimentary to the 3′-end of Exon 1 and bases that are complimentary to the 5′-end of Exon 2. This ASO could bind once the 3D structure is formed (as is shown in FIG. 4B) and disrupt the actual splicing event. The ASO could also bind and effect the entire formation of the 3D structure and its corresponding chemical nature. Numerous other ASO configurations are also envisioned, provided that they induce exon skipping in one more of the exons present. In some embodiments, the ASO overlaps both a portion of the intro and exon both. In other embodiments, the ASO only overlaps an exon portion of the target region. In other embodiments, the ASO only overlaps a portion of the intro near the target region.

Another embodiment is shown in FIG. 4C, where an antibody specific to a given intron/exon junction is added that produces a exon skipping event. Still another embodiment is shown in FIG. 4D, where a small molecule is used to induce the exon skipping event.

Importantly, the molecules described in this patent must be specific to certain portions of a genome and not randomly bind to intro/exon junction regions, which would cause undesirable side effects and potentially disrupt normal biological processes not targeted. In certain embodiments, the molecules act by preventing the formation and stabilization of the 3D structure shown. In other embodiments, the molecules bind to the region and physically or chemically block the cofactors and enzymes from completing the splicing event.

It is contemplated that the methods and compositions disclosed herein will allow ASOs to be more fully studied in animals to understand the various effects of drug dosing in order to further define safe and effective treatment strategy for human patients. It is also contemplated that other factors related to drug dosing (such as weight or disease progression) can be studied in animals, allowing patients to be “stratified” or “categorized” during clinical trials and receive a different dose depending on the particular description of different patients. It is also contemplated that mRNA levels can be monitored in patients as a companion diagnostic during treatment and their dosing can be altered on a patient-by-patient basis based on their molecular response to the drug treatment.

In certain embodiments, the use of morpholino anti-sense oligonucleotides (or other non-charged backbone chemistry oligonucleotides) enhances the strength of the bonding of an ASO once it has hybridized to the target region of the pre-mRNA. Since non-charged backbone structures have no ionic repulsion competing with base pair binding (such as with 2′Me-O ASOs or miRNA drugs), once the ASO is bound, it resists displacement by the transcription proteins (and other cofactors) and leads to significant improvement. Morpholino backbones are well studied in the literature and used by Sarepta Therapeutics for certain FDA approved ASO products for other uses (such as Eteplirsen).

Other non-natural amino acid backbones for the synthesis of ASOs of this disclosure are also contemplated. For example, 2′Me-O modifications to the ASO sugar and backbone can be made in order to stabilize ASOs from enzymatic degradation. Numerous of backbone chemistries, sugar modification, terminal modifications, etc. can also be used in the methods of this disclosure to form a stable ASO having antisense activity (see all of the examples listed in US 2019/0062741 A1). Certain embodiments encompass any ASO structure that hybridizes to the complimentary pre-mRNA to enable exon skipping.

Certain embodiments of this disclosure provide for a composition comprising an antisense oligonucleotide (ASO) that comprises or consists of a complementary region that is complementary, or complementary except for one, two, three, four, or five mismatched nucleotides, to a target region of a PMP22 pre-mRNA. In certain embodiments, the complementary region is 70%, 75%, 80%, 85%, 90%, 95%, 98%, or 99% complementary to a target region of the pre-mRNA. It is understood that the ASO need not be complementary to the full length of the target region but is complementary to a sufficient portion of the target region to hybridize, i.e., the defined target region can be longer than the ASO complementary region and longer than the entire ASO, i.e., the complementary region of the ASO is complementary to a subset of the target region sequence. In certain embodiments, the complementary region and the target region are the same length. As described in greater detail elsewhere herein, binding in a cell of the complementary region of the ASO to the target region of the PMP22 pre-mRNA induces exon skipping during RNA transcription. Binding of the ASO to the target region can reduce full-length PMP22 mRNA production. It can also lead to the production of exon-skipped PMP22 mRNA. Binding of the ASO to the target region can lead to a reduction in functional PMP22 protein and/or production of non-functional PMP22 protein. Both the reduction in full-length mRNA and the production of exon-skipped mRNA can be detected and measured. The reduction in functional protein and/or the production of non-functional protein can also be detected and measured. Further the correlation and/or ratio between full-length and exon-skipped mRNAs and functional and non-functional proteins can be determined and calculated for purposes such as disclosed in detail elsewhere herein.

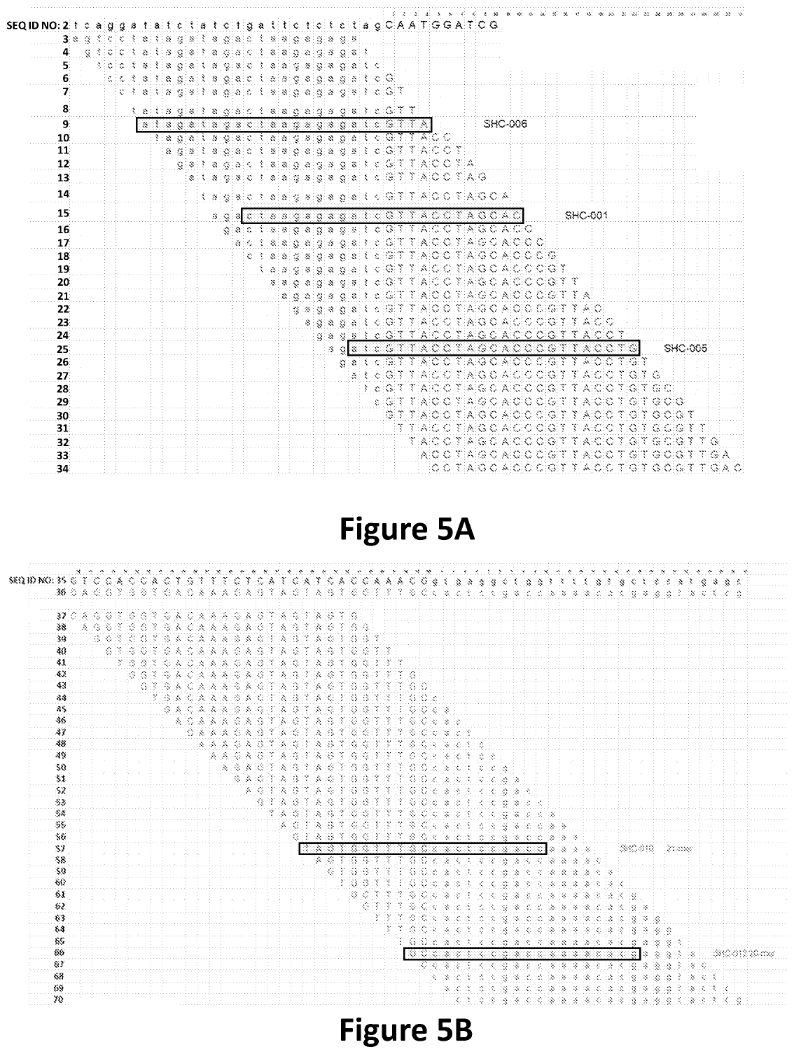

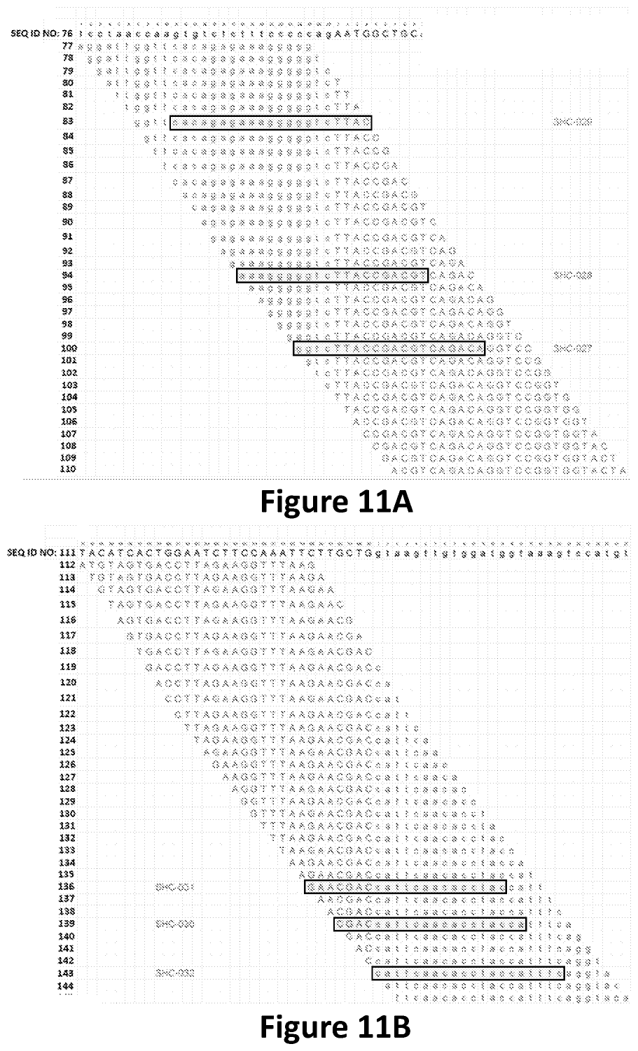

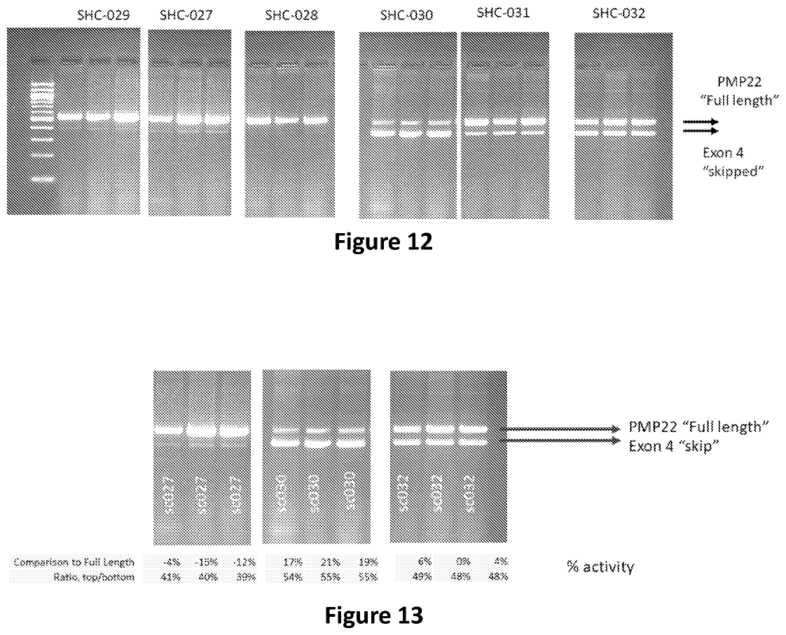

In certain embodiments, the ASO comprises or consists of a complementary region of at least about 12, 13, 14, 15, 16, 17, 18, 19, 20, 21, 22, 23, or 24 contiguous nucleotides that are complementary, or complementary except for one, two, three, four, or five mismatched nucleotides, to the PMP22 pre-mRNA target region. One of ordinary skill in the art would recognize that the length of the ASO's complementary region can vary depending on, for example, the PMP22 pre-mRNA target region sequence and/or the particular application or conditions of administration. Illustrative examples of ASOs with a complementary region of “contiguous nucleotides” are disclosed in Example 1 and Example 4 that follow (e.g., FIG. 5A,B and FIG. 11A,B).

In certain embodiments, the PMP22 pre-mRNA target region comprises two separate segments of the PMP22 pre-mRNA such as described in Example 5. In certain embodiments, the ASO comprises or consists of a complementary region of at least about 6, 8, 9, 10, 11, or 12 nucleotides that are complementary, or complementary except for one, two, or three mismatched nucleotides, to a first segment of contiguous sequence of the PMP22 pre-mRNA target region and the ASO also comprises or consists of a complementary region of at least about 6, 8, 9, 10, 11, or 12 nucleotides that are complementary, or complementary except for one, two, or three mismatched nucleotides, to a second segment of contiguous sequence of the PMP22 pre-mRNA target region. Thus, the complementary region of the ASO hybridizes to the first and second segments of the PMP22 pre-mRNA and also spans a region of the PMP22 pre-mRNA that it is not complementary/does not hybridize to.

In certain embodiments of an ASO of this disclosure, whether the PMP22 pre-mRNA target region is one contiguous segment or two separate segments, the ASO comprises or consists of a complementary region between any of about 12, 13, 14, 15, 16, 17, 18, 19, 20, 21, 22, 23, 24, 25, 30, 35, 40, or 45 and any of about 13, 14, 15, 16, 17, 18, 19, 20, 21, 22, 23, 24, 25, 26, 27, 28, 29, 30, 35, 40, 45, or 50 nucleotides that are complementary, or complementary except for one, two, three, four, or five mismatched nucleotides, to the PMP22 pre-mRNA target region. In certain embodiments, the ASO comprises or consists of a complementary region of 16, 17, 18, 19, 20, 21, 22, 23, 24, or 25 nucleotides that are complementary, or complementary except for one, two, three, four, or five mismatched nucleotides, to the PMP22 pre-mRNA target region.

In certain embodiments of any ASO of this disclosure, whether the pre-mRNA target region is one contiguous segment or two separate segments, the ASO has a length of any of about 8, 9, 10, 11, 12, 13, 14, 15, 16, 17, 18, 19, 20, 21, 22, 23, 24, 25, 30, 35, 40, 45, 50, 60, or 75 and any of about 13, 14, 15, 16, 17, 18, 19, 20, 21, 22, 23, 24, 25, 26, 27, 28, 29, 30, 35, 40, 45, 50, 60, 75, or 100 nucleotides.

In certain embodiments, the ASO is a modified and/or synthetic oligonucleotide as known in the art and/or defined elsewhere herein. For example, the ASO can be a phosphorodiamidate morpholino oligomer (PMO).

Although the ASO causes PMP22 pre-mRNA exon skipping, in certain embodiments this can be done in a manner in which downstream exons are still expressed as they would be from full-length PMP22 pre-mRNA, absent the portion from skipped exon. In certain embodiments, however, the exon skipping forces early termination of protein translation and/or downstream exons to be out of frame.

It has been discovered that exon skipping and the production of exon-skipped PMP22 mRNA has certain advantages over prior methods that do not disclose producing exon-skipped PMP22 mRNA. Thus, in certain embodiments, the target region of the PMP22 pre-mRNA spans an intron/exon junction of at least one of the coding exons (e.g., PMP22 Exon 2, Exon 3, Exon 4, and Exon 5). The targeted intron/exon junction can be at the 3′-end and/or the 5′-end of an exon. For example, in certain embodiments, the target region of the PMP22 pre-mRNA comprises the 3′-end of an exon. And, in certain embodiments the target region of the PMP22 pre-mRNA comprises the 5′-end of an exon. In certain embodiments, the target region of the PMP22 pre-mRNA spans an intron/exon junction comprising or consisting of at least 2, 3, 4, 5, 6, 7, 8, 9, 10, 11, or 12 nucleotides of the intron and a portion of the exon (e.g., FIG. 5A,B and FIG. 11A,B). In certain embodiments, the target region of the PMP22 pre-mRNA consists of 2, 3, 4, 5, 6, 7, 8, 9, 10, 11, or 12 nucleotides of the intron and a portion of the exon. Likewise, in certain embodiments, the target region of the PMP22 pre-mRNA spans an intron/exon junction comprising or consisting of at least 2, 3, 4, 5, 6, 7, 8, 9, 10, 11, or 12 nucleotides of the exon and a portion of the intron (e.g., FIG. 5A,B and FIG. 11A,B). In certain embodiments, the target region of the PMP22 pre-mRNA consists of 2, 3, 4, 5, 6, 7, 8, 9, 10, 11, or 12 nucleotides of the exon and a portion of the intron. In certain embodiments, the exon portion of the intron/exon junction comprises PMP22 Exon 3 (FIG. 5A,B). In certain embodiments, the exon portion of the intron/exon junction comprises PMP22 Exon 4 (FIG. 11A,B).

In certain embodiments, the PMP22 pre-mRNA target region comprises the 5′-end of Exon 3. For example, wherein the PMP22 pre-mRNA target region comprises or consists of SEQ ID NO: 2 or a portion or subset/fragment thereof.

In certain embodiments, the PMP22 pre-mRNA target region comprises the 3′-end of Exon 3. For example, wherein the PMP22 pre-mRNA target region comprises or consists of SEQ ID NO: 35 or a portion or subset/fragment thereof.

In certain embodiments, the PMP22 pre-mRNA target region comprises the 5′-end of Exon 4. For example, wherein the PMP22 pre-mRNA target region comprises or consists of SEQ ID NO: 76 or a portion or subset/fragment thereof.

In certain embodiments, the PMP22 pre-mRNA target region comprises the 3′-end of Exon 4. For example, wherein the PMP22 pre-mRNA target region comprises or consists of SEQ ID NO: 111 or a portion or subset/fragment thereof.

In certain embodiments, the PMP22 pre-mRNA target region comprises the 5′-end of Exon 2. For example, wherein the PMP22 pre-mRNA target region comprises or consists of SEQ ID NO: 163 or a portion or subset/fragment thereof.

In certain embodiments, the PMP22 pre-mRNA target region comprises the 3′-end of Exon 2. For example, wherein the PMP22 pre-mRNA target region comprises or consists of SEQ ID NO: 198 or a portion or subset/fragment thereof.

In certain embodiments, the ASO comprises or consists of a complementary region that is complementary, or complementary except for one, two, three, four, or five mismatched nucleotides, to SEQ ID NO: 2 (Exon 3, 5′-end), SEQ ID NO: 35 (Exon 3, 3′-end), SEQ ID NO: 76 (Exon 4, 5′-end), SEQ ID NO: 111 (Exon 4, 3′-end), SEQ ID NO: 163 (Exon 2, 5′-end), and/or SEQ ID NO: 198 (Exon 2, 3′-end). In certain embodiments, the ASO comprises or consists of a nucleotide sequence of SEQ ID NOs: 3-34 (Exon 3, 5′-end), SEQ ID NOs: 37-70 (Exon 3, 3′-end), SEQ ID NOs: 77-110 (Exon 4, 5′-end), SEQ ID NOs: 112-145 (Exon 4, 3′-end), SEQ ID NOs: 164-197 (Exon 2, 5′-end), or SEQ ID NOs: 199-232 (Exon 2, 3′-end), or a subset/fragment thereof sufficient to hybridize to PMP22 pre-mRNA. In certain embodiments, the ASO comprises or consists of a nucleotide sequence of SEQ ID NOs: 3-34 (Exon 3, 5′-end), SEQ ID NOs: 37-70 (Exon 3, 3′-end), SEQ ID NOs: 77-110 (Exon 4, 5′-end), SEQ ID NOs: 112-145 (Exon 4, 3′-end), SEQ ID NOs: 164-197 (Exon 2, 5′-end), or SEQ ID NOs: 199-232 (Exon 2, 3′-end), except for having one, two, or three nucleotide substitutions, or a subset/fragment thereof sufficient to hybridize to PMP22 pre-mRNA.

In certain embodiments, the ASO comprises or consists of the nucleic acid sequence of: SEQ ID NO: 71 (SHC-006 25-mer), SEQ ID NO: 72 (SHC-001 24-mer), SEQ ID NO: 73 (SHC-005 25-mer), SEQ ID NO: 74 (SHC-010 21-mer), SEQ ID NO: 75 (SHC-012 20-mer), SEQ ID NO: 146 (SHC-029 21-mer), SEQ ID NO: 147 (SHC-028 20-mer), SEQ ID NO: 148 (SHC-027 20-mer), SEQ ID NO: 149 (SHC-031 21-mer), SEQ ID NO: 150 (SHC-030 20-mer), SEQ ID NO: 151 (SHC-032 20-mer), SEQ ID NO: 156, SEQ ID NO: 159, SEQ ID NO: 162, SEQ ID NO: 235, or SEQ ID NO: 238. In certain embodiments, the ASO comprises or consists of the nucleic acid sequence of: SEQ ID NO: 71 (SHC-006 25-mer), SEQ ID NO: 72 (SHC-001 24-mer), SEQ ID NO: 73 (SHC-005 25-mer), SEQ ID NO: 74 (SHC-010 21-mer), SEQ ID NO: 75 (SHC-012 20-mer), SEQ ID NO: 146 (SHC-029 21-mer), SEQ ID NO: 147 (SHC-028 20-mer), SEQ ID NO: 148 (SHC-027 20-mer), SEQ ID NO: 149 (SHC-031 21-mer), SEQ ID NO: 150 (SHC-030 20-mer), SEQ ID NO: 151 (SHC-032 20-mer), SEQ ID NO: 156, SEQ ID NO: 159, SEQ ID NO: 162, SEQ ID NO: 235, or SEQ ID NO: 238, except for having one, two, or three nucleotide substitutions.

Provided for herein is a method of decreasing the amount of full-length PMP22 mRNA expression in a cell comprising administering to the cell a composition comprising an antisense oligonucleotide (ASO) of this disclosure. In certain embodiments, decreasing the amount of full-length PMP22 mRNA comprises targeting a junction between an intron and an exon within a PMP22 pre-mRNA as described in greater detail elsewhere herein. As used anywhere herein, “administering to the cell” is understood to cover all situations where the ASO is placed in contact with the cell in a manner that the cell may take-up the ASO so that the ASO may exert its antisense activity. For example, administering to the cell includes exposing cells in an in vitro experiment to the ASO, such as to cells grown in tissue culture. Administering to the cell also includes providing the ASO to a subject, such as a research animal in an in vivo experiment, such that at least one cell of the subject, through administration locally, systemically, etc., is contacted with the ASO. Administering to the cell also includes providing the ASO to a patient, such as treating a human patient, such that at least one cell of the patient, through administration locally, systemically, etc., is contacted with the ASO. Thus, the subject cell may reside in a tissue, organ, body part, biological fluid, whole organism, and the like.

In certain embodiments of decreasing the amount of full-length PMP22 mRNA, the amount of full-length PMP22 mRNA in the cell is decreased by at least about 5%, 10%, 15%, 20%, 25%, 30%, 40%, 50%, 60%, 75%, or 95% in response to the ASO. The amount of full-length PMP22 mRNA and/or its decrease in any method of this disclosure can be compared against the amount of full-length PMP22 mRNA in an untreated cell, subject, patient, etc., to which the ASO composition has not been administered, such as described in the Examples that follow. In certain embodiments it is not contemplated or desired to fully eliminate full-length PMP22 mRNA (or functional PMP22 protein), and thus the amount of full-length PMP22 mRNA in the cell is decreased by not more than about 25%, 30%, 40%, 50%, 60%, 70%, 80%, 90%, or 95% in response to the ASO. In certain embodiments, the amount of full-length PMP22 mRNA in the cell is decreased by from any of about 5%, 10%, 15%, 20%, 25%, 30%, 40%, 50%, 60%, or 75% to any of about 25%, 30%, 40%, 50%, 60%, 70%, 80%, 90%, or 95% in response to the ASO. As discussed in greater detail elsewhere herein, in certain embodiments, a PMP22 exon-skipped mRNA is produced. Thus, in certain embodiments, the amount/reduction of full-length PMP22 mRNA can be compared against the amount of PMP22 exon-skipped mRNA to determine a correlation, calculate a ratio, etc. In certain embodiments, the amount of functional PMP22 protein produced in the cell is decreased. This decrease in functional protein in any method of this disclosure can be determined against an untreated control cell, subject, patient, etc. to which the ASO composition has not been administered. In certain embodiments, the amount of functional PMP22 protein in the cell is decreased by at least about 5%, 10%, 15%, 20%, 25%, 30%, 40%, 50%, 60%, 75%, or 95% in response to the ASO. As noted, in some embodiments it is not contemplated or desired to completely eliminate PMP22 protein. Thus, in some embodiments, the amount of functional PMP22 protein in the cell is decreased by not more than about 25%, 30%, 40%, 50%, 60%, 70%, 80%, 90%, or 95% in response to the ASO. In certain embodiments, the amount of functional PMP22 protein in the cell is decreased by from any of about 5%, 10%, 15%, 20%, 25%, 30%, 40%, 50%, 60%, or 75% to any of about 25%, 30%, 40%, 50%, 60%, 70%, 80%, 90%, or 95% in response to the ASO.

Provided for herein is a method of producing an exon-skipped PMP22 pre-mRNA comprising administering to a cell a composition comprising an antisense oligonucleotide (ASO) of this disclosure. In certain embodiments, the method comprises targeting a junction between an intron and an exon within a PMP22 pre-mRNA as described in greater detail elsewhere herein. In certain embodiments, the amount of full-length PMP22 mRNA expression in the cell is decreased. In certain embodiments, the amount of full-length PMP22 mRNA in the cell is decreased by at least about 5%, 10%, 15%, 20%, 25%, 30%, 40%, 50%, 60%, 75%, or 95% in response to the ASO. In certain embodiments it is not contemplated or desired to fully eliminate full-length PMP22 mRNA (or functional PMP22 protein), and thus the amount of full-length PMP22 mRNA in the cell is decreased by not more than about 25%, 30%, 40%, 50%, 60%, 70%, 80%, 90%, or 95% in response to the ASO. In certain embodiments, the amount of full-length PMP22 mRNA in the cell is decreased by from any of about 5%, 10%, 15%, 20%, 25%, 30%, 40%, 50%, 60%, or 75% to any of about 25%, 30%, 40%, 50%, 60%, 70%, 80%, 90%, or 95% in response to the ASO. In certain embodiments, the amount/reduction of full-length PMP22 mRNA can be compared against the amount of PMP22 exon-skipped mRNA produced to determine a correlation, calculate a ratio, etc. Further, in certain embodiments, the amount of functional PMP22 protein produced in the cell is decreased. In certain embodiments, the amount of functional PMP22 protein in the cell is decreased by at least about 5%, 10%, 15%, 20%, 25%, 30%, 40%, 50%, 60%, 75%, or 95% in response to the ASO. As noted, in some embodiments it is not contemplated or desired to completely eliminate PMP22 protein. Thus, in some embodiments, the amount of functional PMP22 protein in the cell is decreased by not more than about 25%, 30%, 40%, 50%, 60%, 70%, 80%, 90%, or 95% in response to the ASO. In certain embodiments, the amount of functional PMP22 protein in the cell is decreased by from any of about 5%, 10%, 15%, 20%, 25%, 30%, 40%, 50%, 60%, or 75% to any of about 25%, 30%, 40%, 50%, 60%, 70%, 80%, 90%, or 95% in response to the ASO.

Provided for herein is a method of treating Charcot-Marie-Tooth disease, such as Charcot-Marie-Tooth disease type 1A, comprising administering to a subject in need thereof a composition comprising an antisense oligonucleotide (ASO) of this disclosure. In certain embodiments, the subject is a model system for Charcot-Marie-Tooth disease such as a research animal. In certain embodiments, the subject is a human patient. In certain embodiments, the composition is administered orally, locally, systemically, e.g., subcutaneously, perineurally, etc. In certain embodiments, the method comprises targeting a junction between an intron and an exon within a PMP22 pre-mRNA as described in greater detail elsewhere herein. In certain embodiments, a PMP22 exon-skipped mRNA is produced and in certain embodiments can be compared to the amount/decrease in full-length PMP22 mRNA as described in detail elsewhere herein. In certain embodiments, the amount of full-length PMP22 mRNA in the cell is decreased. In certain embodiments, the amount of full-length PMP22 mRNA in the cell is decreased by at least about 5%, 10%, 15%, 20%, 25%, 30%, 40%, 50%, 60%, 75%, or 95% in response to the ASO. In certain embodiments it is not contemplated or desired to fully eliminate full-length PMP22 mRNA (or functional PMP22 protein), and thus the amount of full-length PMP22 mRNA in the cell is decreased by not more than about 25%, 30%, 40%, 50%, 60%, 70%, 80%, 90%, or 95% in response to the ASO. In certain embodiments, the amount of full-length PMP22 mRNA in the cell is decreased by from any of about 5%, 10%, 15%, 20%, 25%, 30%, 40%, 50%, 60%, or 75% to any of about 25%, 30%, 40%, 50%, 60%, 70%, 80%, 90%, or 95% in response to the ASO. In certain embodiments, the amount of functional PMP22 protein in the cell is decreased. In certain embodiments, the amount of functional PMP22 protein in the cell is decreased by at least about 5%, 10%, 15%, 20%, 25%, 30%, 40%, 50%, 60%, 75%, or 95% in response to the ASO. As noted, in some embodiments it is not contemplated or desired to completely eliminate PMP22 protein. Thus, in some embodiments, the amount of functional PMP22 protein in the cell is decreased by not more than about 25%, 30%, 40%, 50%, 60%, 70%, 80%, 90%, or 95% in response to the ASO. In certain embodiments, the amount of functional PMP22 protein in the cell is decreased by from any of about 5%10%, 15%, 20%, 25%, 30%, 40%, 50%, 60%, or 75% to any of about 25%, 30%, 40%, 50%, 60%, 70%, 80%, 90%, or 95% in response to the ASO.

In certain embodiments, the ASO is administered as a pharmaceutically acceptable salt. In certain embodiments, the ASO is administered in a pharmaceutically acceptable carrier or diluent.

In certain embodiments, at least one symptom of the disease is alleviated. In certain embodiments, the rate of progression of at least one symptom of the disease is decreased. In certain embodiments, the method of treatment results in no side-effects or fewer or less severe side-effects in comparison to other CMT treatments. In certain embodiments, the correlation or ratio between the amount of exon-skipped PMP22 mRNA produced and the amount/reduction of full-length PMP22 mRNA can be used to adjust the dosage of the ASO treatment to increase its effectiveness and/or to decrease side-effects.

Also provided for herein is a composition comprising an antisense oligonucleotide (ASO) comprising or consisting of a complementary region that is complementary, or complementary except for one, two, three, four, or five mismatched nucleotides, to a target region of a PMP22 pre-mRNA, wherein the target region of the PMP22 pre-mRNA comprises an intron/exon junction of one of the coding exons (i.e., PMP22 Exon 2, Exon 3, Exon 4, or Exon 5). It is understood that the ASO need not be complementary to the full length of the target region but is complementary to a sufficient portion of the target region to hybridize, i.e., the defined target region can be longer than the ASO complementary region and longer than the entire ASO. In certain embodiments, the ASO comprises or consists of a complementary region of at least about 12, 13, 14, 15, 16, 17, 18, 19, 20, 21, 22, 23, or 24 contiguous nucleotides that are complementary, or complementary except for one, two, three, four, or five mismatched nucleotides, to the PMP22 pre-mRNA target region. In certain embodiments, the ASO comprises or consists of a complementary region between any of about 12, 13, 14, 15, 16, 17, 18, 19, 20, 21, 22, 23, 24, 25, 30, 35, 40, or 45 and any of about 13, 14, 15, 16, 17, 18, 19, 20, 21, 22, 23, 24, 25, 26, 27, 28, 29, 30, 35, 40, 45, or 50 contiguous nucleotides that are complementary, or complementary except for one, two, three, four, or five mismatched nucleotides, to the PMP22 pre-mRNA target region. In certain embodiments, the ASO comprises or consists of a complementary region of 16, 17, 18, 19, 20, 21, 22, 23, 24, or 25 contiguous nucleotides that are complementary, or complementary except for one, two, three, four, or five mismatched nucleotides, to the PMP22 pre-mRNA target region. In certain embodiments, the target region of the PMP22 pre-mRNA comprises the 3′-end of an exon. In certain embodiments, the target region of the PMP22 pre-mRNA comprises the 5′-end of an exon. In certain embodiments, the target region of the PMP22 pre-mRNA comprises an intron/exon junction comprising at least 2, 3, 4, 5, 6, 7, 8, 9, 10, 11, or 12 nucleotides of the intron and a portion of the exon. In certain embodiments, the target region of the PMP22 pre-mRNA comprises or consists of 2, 3, 4, 5, 6, 7, 8, 9, 10, 11, or 12 nucleotides of the intron and a portion of the exon. Likewise, in certain embodiments, the target region of the PMP22 pre-mRNA comprises an intron/exon junction comprising at least 2, 3, 4, 5, 6, 7, 8, 9, 10, 11, or 12 nucleotides of the exon and a portion of the intron. In certain embodiments, the target region of the PMP22 pre-mRNA comprises or consists of 2, 3, 4, 5, 6, 7, 8, 9, 10, 11, or 12 nucleotides of the exon and a portion of the intron. In certain embodiments, the exon portion of the intron/exon junction comprises PMP22 Exon 3. In certain embodiments, the exon portion of the intron/exon junction comprises PMP22 Exon 4. In certain embodiments, the ASO is a modified and/or synthetic oligonucleotide as known to those of ordinary skill in the art and/or as disclosed herein. For example, in certain embodiments, the ASO is a phosphorodiamidate morpholino oligomer (PMO). In certain embodiments, the PMP22 pre-mRNA target region comprises or consists of SEQ ID NO: 2 (Exon 3, 5′-end), SEQ ID NO: 35 (Exon 3, 3′-end), SEQ ID NO: 76 (Exon 4, 5′-end), SEQ ID NO: 111 (Exon 4, 3′-end), SEQ ID NO: 163 (Exon 2, 5′-end), and/or SEQ ID NO: 198 (Exon 2, 3′-end).

In certain embodiments, a tailored method of treatment can be used for patients that depends on the age of the patient and severity of the symptoms (e.g., Example 9). In younger subjects and/or when symptoms are not severe, small doses of the compositions of this disclosure can have a positive and complete impact on axon improvement. However, as shown in Example 9 for example, the 5 mg/kg group showed only a small amount of improvement at the 30 day mark compared to the 50 mg/kg groups (2 and 4) that were tested at day 30. But, after the second treatment of Group 5 and subsequent testing at 60 days, the dowel walking time was greatly improved. This indicates that older subjects and/or subjects with greater levels of symptoms or disease progression time will likely require either their dosing to be higher than other patients or a loading dose regiment (where injections are given a few time in the first few months of treatment followed by a standard course of treatment after that) may be desirable. For other subjects where the disease has not progressed, a loading dose period may not be required. From a biological perspective, this observation is very surprising, since the PMP22 mRNA levels (as shown in Table 4) seem to be equally effective no matter what dosing is given once the animals have normalized for at least 30 days. It does appear that animals tested and sacrificed after only 1 week (Group 1) have not yet had time to normalize their PMP22 mRNA levels. Additionally, Group 1 showed no improvement in performance which indicates the myelin sheaths surrounding the axons have not yet improved even though the PMP22 levels have been effected.

Therefore, certain embodiments provide for prescribing a dosing regimen on a subject-to-subject basis based on their symptom progression or age. In certain embodiments, the treatment amount of a composition of this disclosure administered to a subject is based both on their disease progression and overall body weight. In another embodiment, the disease progression is based on age of the patient, or the physical performance of the patient, or both. In another embodiment, subjects with greater symptom severity and/or older subjects are given a loading dose of the treatment described for a period of time which is then later changed to a lower dose. The loading period can be the same amount of drug given to the subject simply more often, or it can be a larger dose of the composition initially administered. After a period of time (which may be based on time or may be based on physical improvements or some biomarker improvement), the dosing amount and/or dosing frequency can be lowered.

### EXAMPLES

### Example 1—Skipping of Exon 3

A number of compounds were designed (FIG. 5) to bind to the PMP22 pre-mRNA and promote exon skipping (FIG. 2). Some of these compounds were designed to interact with the 5′-end of the intron/exon region while other compounds interact with the 3′-region. The target regions within the PMP22 pre-mRNA are shown at the top of FIG. 5A and FIG. 5B while the specific 25-mer ASOs described are shown below. Nucleotide bases are shown in lower case (for intron regions) and upper case for protein coding exon regions (for example, g and G are identical structures). These compounds were designed to bind to the pre-mRNA and induce skipping of Exon 3. While the designed ASOs in FIG. 5 are 25 base pairs long, one of ordinary skill in the art would understand the length of the complementary region of the ASO can vary and be either longer or shorter and still be effective as long as the ASO is able to specifically hybridize to the target region of PMP22 pre-mRNA. For example, some of the ASOs tested were of shorter length. In FIG. 5A, the sequence of PMO SHC-001 (SEQ ID NO: 72) is ctaagagagatcGTTACCTAGCAC, which is 22 base pairs long and a subset of the 25-mer (SEQ ID NO: 15) shown in FIG. 5.

A number of these compounds were synthesized with morpholino backbone chemistry and were studied both in cellular assays and mouse models to determine their effectiveness at blocking PMP22 mRNA production and producing a measurable exon-skipped product to confirm mode of action and activity. As noted above, the length of these compounds varied (not all compounds were the full 25 bases long, some were subsets of the 25 base pair ASO shown).

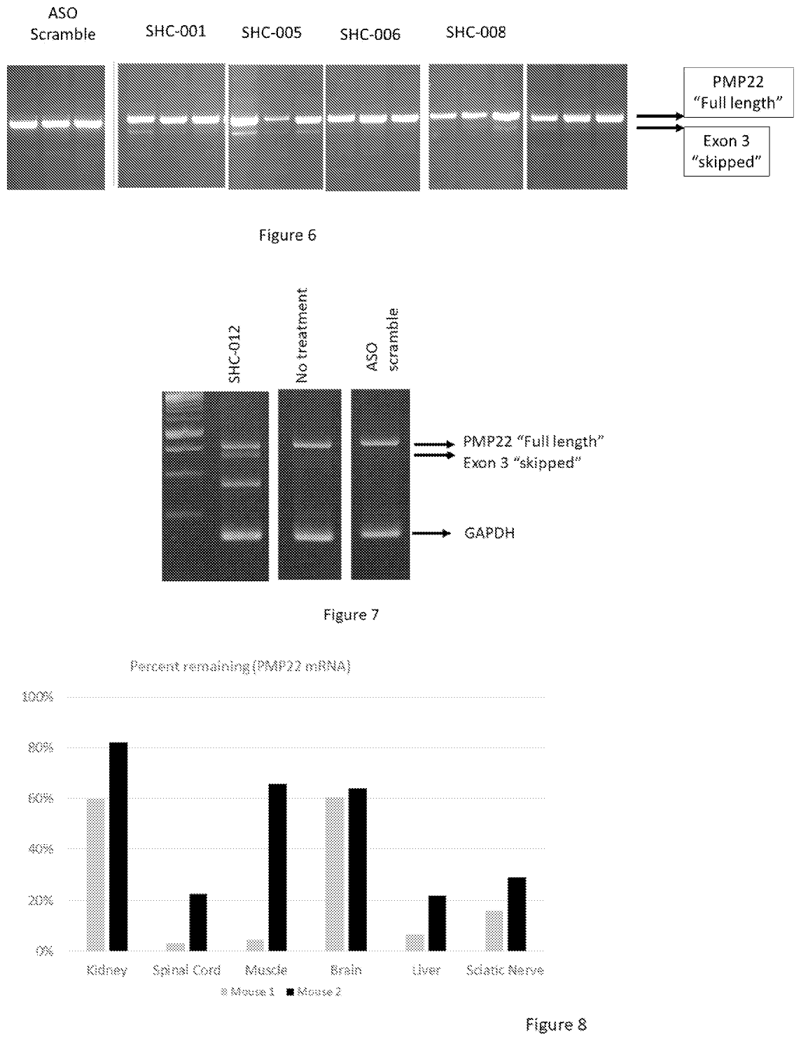

Compounds from FIG. 5 were screened in HEK293 cellular assays where the cells express the human PMP22 gene. Cells were grown and the test compounds were added. After a 48-hour incubation period, transfected cells were harvested for total RNA for end-point RT-PCR. The results from the cellular assays for compounds SHC-006 (SEQ ID NO: 71), SHC-001 (SEQ ID NO: 72), SHC-005 (SEQ ID NO: 73), SHC-010 (SEQ ID NO: 74), SHC-012 (SEQ ID NO: 75) are shown in FIG. 6.

Each of these compounds effectively reduced the total amount of full-length PMP22 mRNA (and thus will lower the amount of PMP22 protein produced) and produces a stable exon-skipped mRNA product that has been confirmed by sequencing to contain the exact sequence of PMP22 full length mRNA minus the sequences of Exon 3. Similar results were also obtained using two additional cellular contexts, K-562 cells (myelogenous leukemia cell line) and CLR-3392 cells, which are a human Schwann cell line (data not shown). These cells do not transfect nearly as well as the HEK293 cells, but allowing for reduced transfection efficacy, similar levels of exon-skipping were observed.

Schwann cells are the primary producer of PMP22 and are therefore the primary target in any therapeutic envisioned for CMT1A. Schwann cells are heavily myelinated, and their biochemistry is greatly affected by their surrounding tissues, making cellular studies (while feasible) quite challenging as compared to some other target cells. However, disclosed herein is an optimized method to do this and developed a robust assay for inducing PMP22 exon-skipping in the pre-mRNA.

The DNA of both the full-length mRNA and exon-skipped mRNA gives a well-defined molecular read-out that allows examination of “target engagement” and molecular efficacy with a relatively straightforward RT-PCR assay: inducing exon-skipping of exon 3 from the human PMP22. This is advantageous from a drug development perspective as a reliable target is available that can be readily monitored in an internally controlled RT-PCR reaction for the concomitant decrease of full-length PMP22 and the increase of exon-skipped PMP22.

### Example 2

In vivo activity was tested using C3 mice (3 copies of human PMP22). C3 mice were bred and genotyped according to the published protocol from Jackson Labs. At 5 weeks, animals were injected subcutaneously with a single 6.7 mg of SHC-012 (SEQ ID NO: 75) or a scramble control PMO. Untreated C3 mice were included as a control. Animals were sacrificed after 24 hours after the injection and tissues were collected (sciatic nerve, kidney, liver, brain, spinal cord, etc.) and analyzed for PMP22 mRNA reduction/expression and the exon skipped version of the PMP22 mRNA. RT-PCR was used to evaluate PMP22 expression (3% agarose gel). GAPDH was included in the reaction as a control to allow for comparison between lanes (FIG. 7). As the liver will naturally receive a large dose of any peripherally delivered therapeutic, we examined liver PMP22 expression and found the accumulation of exon-skipped PMP22 products with a reduction in full-length PMP22 expression (FIG. 7). The SHC-012 animal has a significant reduction in PMP22 full length mRNA and a measurable amount of exon3 skipped PMP22 mRNA (confirmed by sequencing).

Additionally, other tissues from these animals were analyzed by the same PCR methods and gel analysis (FIG. 8) and the inventors compared the full-length and skipped-exon amounts to determine overall reduction in these tissues. The evidence demonstrates PMP22 reduction and/or exon-skipping in several tissues, including peripheral nervous tissues (sciatic nerve) from C3 mice with a single injection (in the small of the back).

Importantly, when peripheral nervous tissue was examined (e.g., surrogate for in vivo Schwann cells), a clear reduction in full-length PMP22 expression was observed. It is noted that the C3 mice express the human genomic PMP22 gene which allows ASOs to target important regulatory sequences in and around the various exons.

### Example 3—Longer Term Animal Study

C3 mice (3× human PMP22 gene) were selected as the animal model since they have been shown to exhibit CMT like behaviors, have been well studied, and most importantly for this project, express the human genomic PMP22 gene (Huxley C, Passage E, Manson A, Putzu G, Figarella-Branger D, Pellissier J F, Fontes M. Construction of a mouse model of Charcot-Marie-Tooth disease type 1A by pronuclear injection of human YAC DNA. Human molecular genetics. 1996; 5(5):563-9. Epub 1996/05/01. doi: 10.1093/hmg/5.5.563. PubMed PMID: 8733121). This is essential since the ASOs rely on sequence identity within the exon and intronic regions.

Two animal groups were studied: 5-week old animals (early symptomatic disease stage exhibiting partial behavioral changes already) and 3-day old animals (that received treatment prior to measurable CMT behavioral changes). SCH-012 was injected for all animals. At 5 weeks, C3 animals (n=3) were injected subcutaneously with 3.3, 17, or 50 mg/kg of SCH-012 or a scramble control PMO once/week for 5 weeks. A second set of C3 mice were also tested where the initial injections were performed (SQ on the upper back) starting at 3 days of age (to simulate prior to disease onset in humans) with weekly injections occurring equal to 1 mg/kg and 10 mg/kg per animal (for once/week for 12 weeks). Untreated (n=4) WT mice were included as a control and tested as the others. No adverse reactions were observed, and all animals remained healthy through the study period, gaining weight at similar rates throughout all dose ranges and showed no overt signs of toxicity. As anticipated, all animals recovered smoothly, rapidly and without complication from each injection and showed no immediate or delayed ambulatory, behavioral or neurological defects from the injection procedure.

To allow for a training and acclimation period, all animals started testing procedures at 4 weeks of age (˜×4 per week) using a standard rotarod mechanical system and a dowel walk one week prior to initial injection. For the dowel walk, a cylindrical 10 mm wooden dowel was suspended and the mice allowed to walk (unaided and undirected) from one end of the setup to the other. The time to traverse, number of slips during the walk, and any falls during the walk were recorded. The initial training period was not used for data analysis. After injection, animals continued to undergo daily testing to monitor overall balance, leg performance, tail stability, etc. which have been shown to be measurable phenotypic responses to disease progression in this model (Huxley C, Passage E, Manson A, Putzu G, Figarella-Branger D, Pellissier J F, Fontes M. Construction of a mouse model of Charcot-Marie-Tooth disease type 1A by pronuclear injection of human YAC DNA. Human molecular genetics. 1996; 5(5):563-9. Epub 1996/05/01. doi: 10.1093/hmg/5.5.563. PubMed PMID: 8733121). As previously reported, untreated animals began to exhibit very noticeable walking problems between 6-8 week of age.

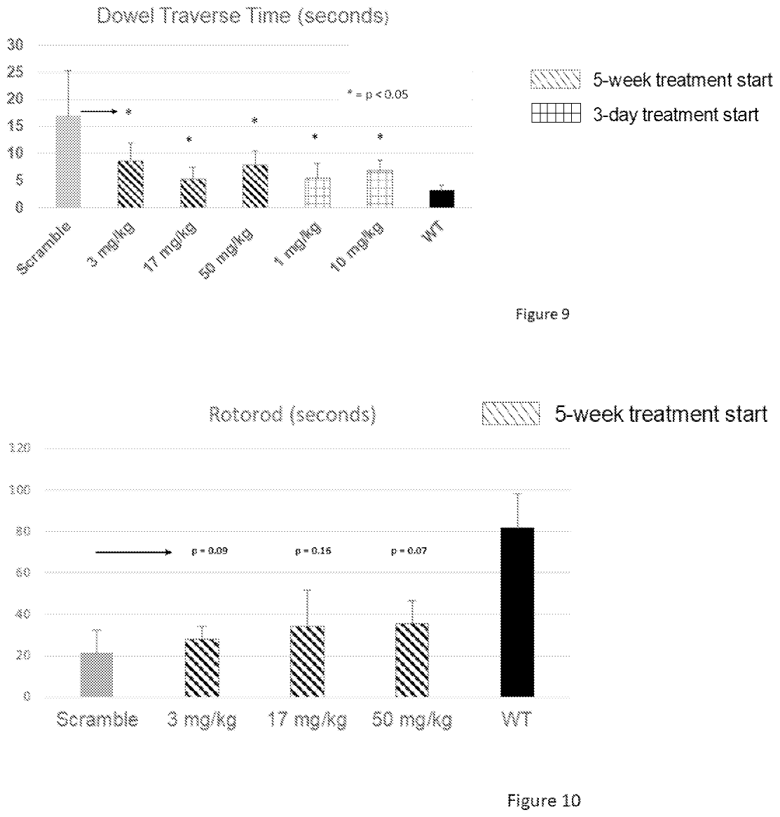

All of the data (from both test groups) was analyzed at 12-weeks of age for all animals for direct comparison, to determine the time required to traverse the dowel, as well as the number of slips, falls, hesitation, etc. Untreated animals learned to traverse more slowly over time and had less slips as the experiments progressed but visibly were walking more carefully often “hugging” the dowel. Video evidence demonstrated a very clear difference between treated and untreated animals with noticeable behavioral differences. FIG. 9 shows the performance (phenotypic) data for animals studied as compared to control and wild-type by comparing the amount of time required to walk across the dowel unaided. The number of slips (and falls) on the dowel showed a similar trend. Five consecutive testing days were analyzed at ˜12-weeks of age to compute averages and standard deviations across all animals within the dosing group for time across the dowel and number of slips. Rotarod testing was also performed but showed a much higher variance per test. The selected concentrations were initially chosen (based on the cellular data and our previous experience with biodistribution of similar molecules) to achieve a low dose (3 mg/kg), medium dose (17 mg/kg), and high dose (50 mg/kg), with the understanding that completely eliminating PMP22 would be deleterious. All animals performed significantly better than the control group (also highly noticeable from the video data) with the 17 mg/kg (5 week) and both groups of animals at P3 performing near wild-type animals.

Animals throughout the study were also tested on a commercial rotarod system and the performance data is shown in FIG. 10.

For both experiments, the p values were calculated (versus the scramble control animal) and are shown in FIG. 9 and FIG. 10.

The data from Examples 2 and 3 clearly indicate the alteration of the PMP22 mRNA by SCH-012 as described in this disclosure has the desired phenotypic effect of the animals used. Namely to make them walk better and alleviate certain symptoms caused by their overproduction of PMP22 protein.

Surprisingly, this approach shows a much greater effectiveness at alleviating symptoms in CMT1A mouse models than the traditional knockdown approach. As an example, referring to FIG. 1D of the paper by Zhao etal. (The Journal of Clinical Investigation, Volume 128, No 1, January 2018, pp 359-368), weekly injections of 100 mg/kg of their most potent ASO which targets the 3′ UTR of the human PMP22 gene into C22 humanized CMT1A mouse models (containing 7 copies of the human PMP22 gene) were required to obtain statistically significant results in rotarod testing. No statistical difference was determined was obtained with 50 mg/kg per week. For the present analysis of animals studied starting at the same age, significant improvement was noted with animals injected as low as 3.3 mg/kg per week. For animals that started treatment at a younger age, only 1 mg/kg was required (see FIG. 9 and FIG. 10). While the C3 animal model used here has only 3 copies of the PMP22 gene and the C22 animals had 7 copies (a 2.3 fold increase of target RNA), 50 mg/kg is 15 fold higher than 3.3 mg/kg and 50 fold higher than the 1 mg/kg animals. Clearly, the exon skipping approach described here has a significant advantage over the 3′ UTR strategy in terms of efficacy per dose.

Improving the critical disease-associated pathology in axons is an important surrogate marker of efficacy. To determine if the molecular change observed in PMP22 resulted in an improvement in axonal pathology, a variety of axons from wild-type were examined, treated, and scramble-treated C3 mice. Animals from the 17 mg/kg treatment group, wild-type animals, and scramble control group C3 animals were sacrificed at 20 weeks of age (6 weeks after the treatment stopped for the 17 mg/kg treatment group above. Nerves were removed from all of the animals (sciatic, peroneal, ulnar, and tibial nerves) and cross-sectional portion of the nerves were prepared for Transmission Electron Microscopy (TEM) analysis.

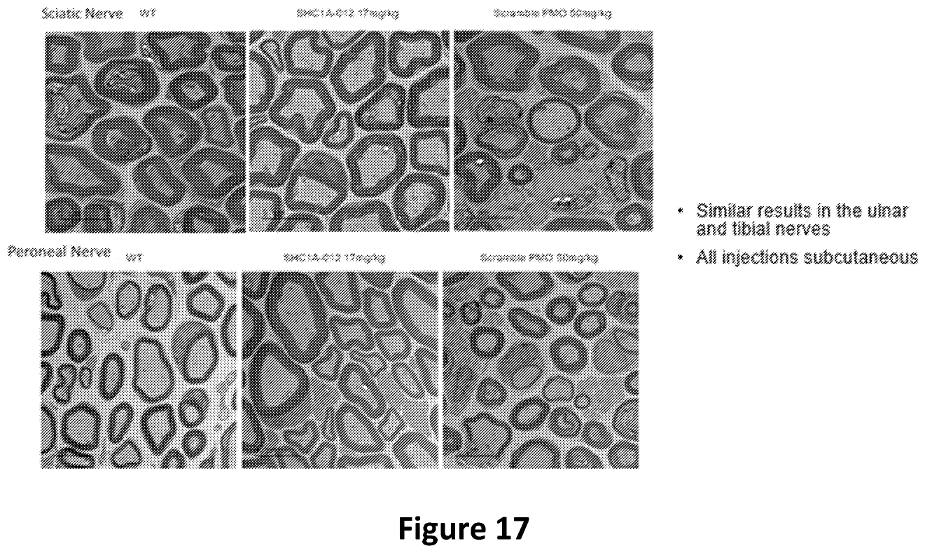

FIG. 17 (top) shows images of the sciatic nerve for WT, untreated and treated animals; FIG. 17 (bottom) shows images from the peroneal portion of the nerve.

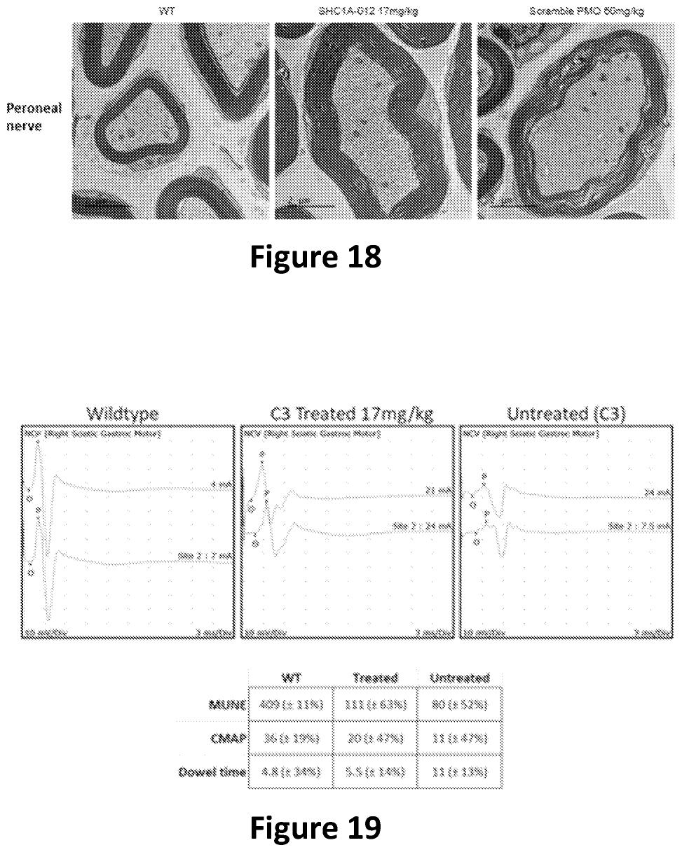

FIG. 18 is a TEM image at higher magnification of portions of a Peroneal nerve from each animal.

Analysis of the sections revealed that SHC-012 treatment significantly improved the pathology from multiple nerves (sciatic is shown) compared to scramble-treated axons based upon demyelination, thickness, structural integrity and the frequency of dying axons. Wild-type tissues were still better than the SHC-012 treated axons, however, it is clear that the ASO was improving these important structural hallmarks of disease. Similar results were also seen when sections from the ulnar and tibial nerves were analyzing, indicating that the subcutaneous injections used (on the scruff of the animals backs) were effectively distributing the molecules throughout the animal's bodies sufficiently to have a positive impact.

Based upon the improvement in the myelin sheath, the functional impact of this improvement was next examined. Electrophysiology was performed to measure functional recovery in C3-treated mice. In this cohort of treated C3 mice (and untreated C3 mice as a control) from the same treatment group described above, improvements in the CMAP and MUNE were detected in the treated mice (FIG. 19).

In FIG. 19 (top), electrophysiology plots from the sciatic gastric section of sedated mice are shown. The table at the bottom of FIG. 19 shows the average values of MUNE and CMAP from 3 measured animals per group. The “dowel time” which is a measure of general fitness, balance and mobility (described below) is included and illustrates that improved dowel performance tracks with improved electrophysiological measures.

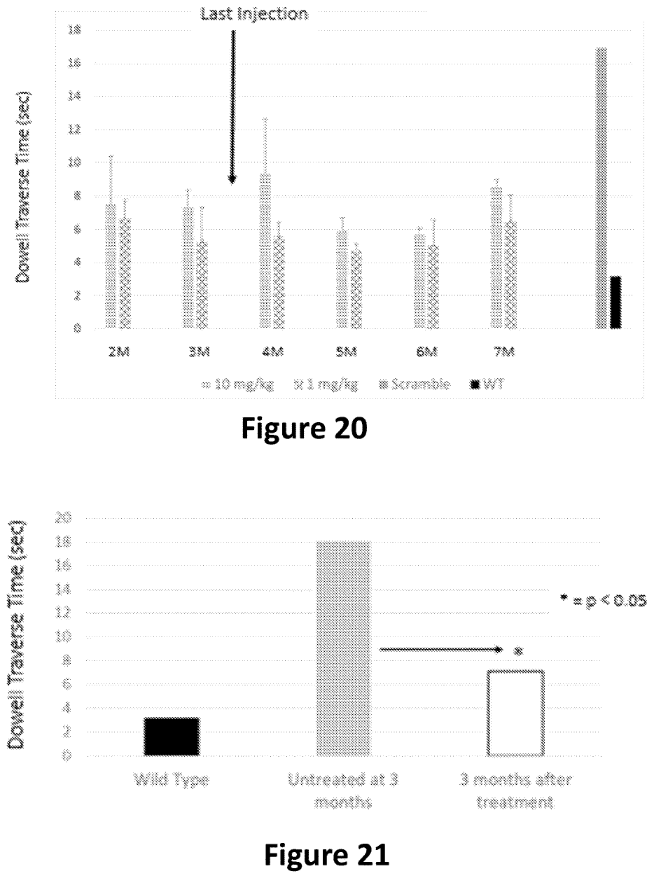

A key specification of the envisioned CMT1A treatment is that treatment will only be required 2-4 times per year for each patient. This is potentially possible because PMO ASOs are resistant to nucleases and are very stable once cellular uptake has occurred. To examine the duration and durability of SHC-012 activity, animals from the 1 mg/kg treatment and 10 mg/kg treatment groups above were monitored for 5 months after their last treatment (FIG. 20). The half-life of PMO molecules (once they reach cells such as Schwann cells) is ˜3-4 months for most tissue; thus, our hypothesis was that treatment benefit would persist for extended periods of time. Remarkably, the C3 animals at 5 months post-treatment have continued to perform well on the dowel test, with little or no reduction in activity after 4 months from last treatment (extended testing is still ongoing).

FIG. 20 shows the results for both treatment groups (with the scramble animals' group and wild-type animals shown for comparison at 4 months of age). Each data set of the histogram is an average of 4 days dowel travers time at the end of each month. Thus, the last data set plotted is for animals that are 7 months old.

### Example 4

A number of ASOs were designed for exon skipping of Exon 4 in the PMP22 RNA (FIG. 11A and FIG. 11B). 25 base pair long ASOs are shown but shorter subsets are also envisioned and disclosed. Both 5′-end and 3′-end region ASOs were designed. Referring to FIG. 3, these ASOs were designed at the 3′- and 5′-end of Exon 4. Select ASOs were tested in cellular assays and the results shown in FIG. 12 (SHC-027 (SEQ ID NO: 148), SHC-028 (SEQ ID NO: 147), SHC-029 (SEQ ID NO: 146), SHC-030 (SEQ ID NO: 150), SHC-031 (SEQ ID NO: 149), and SHC-032 (SEQ ID NO: 151)). Significant amount of Exon 4 skipped product was observed (as well as reduction in full length PMP22 mRNA) and was confirmed by sequencing.

It was important to be able to quantitate the presence of both the exon-skipped product and full-length PMP22 (FIG. 12). The bands within the gel were analyzed and the resulting calculation of “activity” of the compounds was determined using two methods (FIG. 13). In the first method, the amount of full-length PMP22 mRNA in the standard samples (not shown on this figure) was compared to the amount of resulting PMP22 full-length mRNA. This method is the only method possible for knockout and RNA silencing approaches as previously described (since there is only 1 measurable mRNA present in the sample). Due to variance in this experiment, the data for this method produces the results shown in FIG. 13 in the first row at the bottom, namely, SHC-027 had a negative % activity and SHC-032 had very low activity (<10%). Both of these compounds would have been incorrectly identified as being non-effective or only slightly effective. However, by comparing the amount of full-length mRNA to the amount of exon-skipped mRNA (the lower bands in the gel confirmed by sequencing), we were able to very accurately identify the relative activity of compounds (these have been adjusted for differences in sequence length of the two mRNAs). Also, it can be noted that even though each lane of the gel is a completely separately run cellular assay (these are note multiple injections from a single well, rather injections from different assays), the standard deviation of this method is much tighter as compared to the previously described methods of lowering PMP22 production. For the quantitation on the top row (comparison to full-length), the value is simply the measure peak divided by the full-length standard peak. For the Ratio method, the exon-skipped peak is divided by the same of the total mRNA adjusted to size.

### Example 5

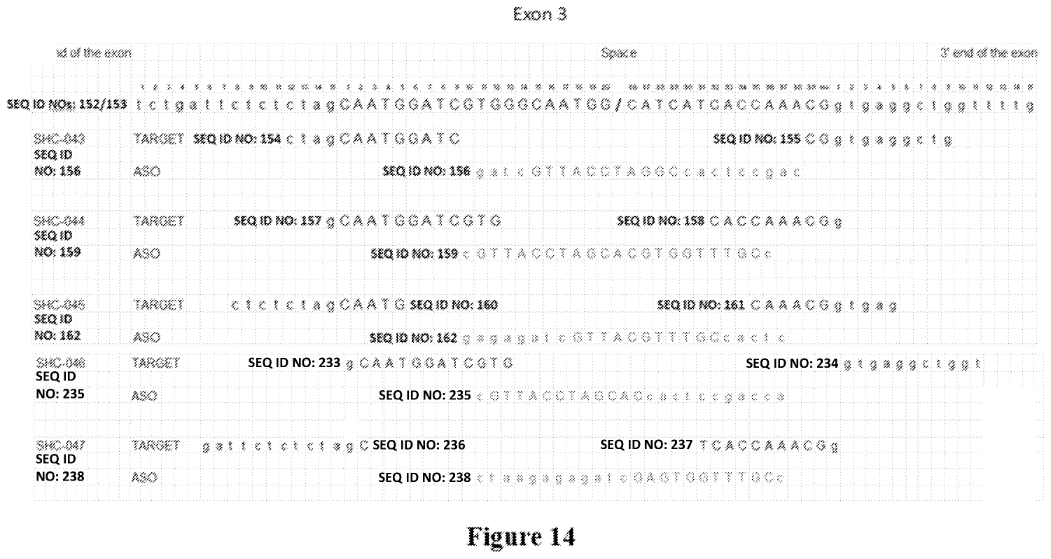

A number of phosphorodiamidate morpholino oligomer PMOs (using morpholino backbones) were designed and synthesized that did not contain a continuous sequence against the PMP22 pre-mRNA, but rather contained bridging portions as described above. FIG. 14 shows the sequence of the 5′- and 3′-ends of Exon 3 at the top, along with the intro portions that are adjacent to the Exons. A gap appears in the top (with the word Space and “/” below) indicating that the other portions of Exon 3 are not shown that would join position 20 (a G) with position 86 (an C). PMOs SHC-043 (SEQ ID NO: 156), SHC-044 (SEQ ID NO: 159), and SHC-045 (SEQ ID NO: 162) were designed as shown against portions of both the 3′- and 5′-ends. These PMOs are continuous molecules (as seen in the FIG. 14) but will hybridize to non-contiguous portions of the PMP22 pre-mRNA. Other bridging ASOs are also envisioned, provided they can hybridize to PMP22 pre-mRNA and induce both the production of an exon-skipped mRNA product and reduction of the full-length mRNA.

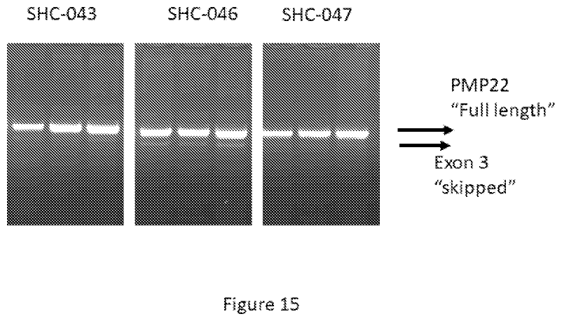

These compounds were used in cellular assays (as described above) to test their performance and the results are shown in FIG. 15, again showing both the full-length and exon-skipped product that can be separately quantitated to determine activity.

### Example 6

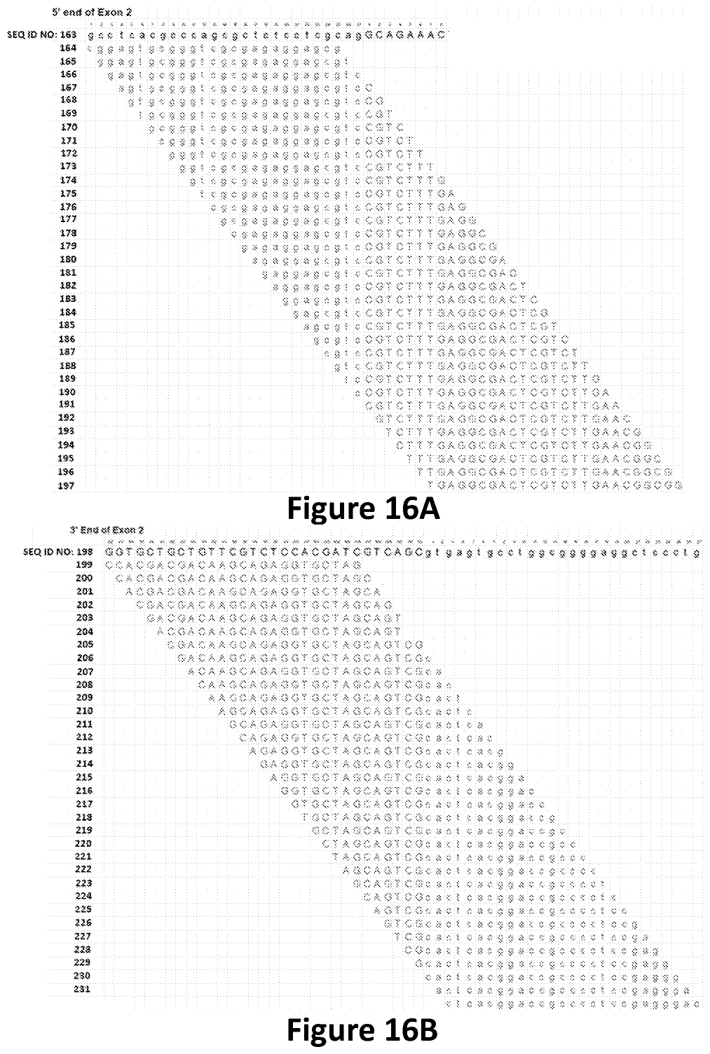

A number of ASOs were designed for exon skipping of Exon 2 in the PMP22 pre-mRNA (FIG. 16). As disclosed elsewhere herein, shorter length and mis-matched ASOs are also contemplated. These ASOs were designed at the 3′- and 5′-end of Exon 2.

### Example 7

For CMT1A, it is desirable to monitor the activity at a molecular level of a given drug over time without having to sacrifice the animal (during research studies) or perform a major biopsy on a human patient, in order to access the tissue of interest and determine if the pre-mRNA, mRNA, or protein for PMP22 has been effected. CMT1A is a disease where the peripheral nerve tissue is the desired tissue to affect the level of PMP22 and within this tissue the myelinated sheets around the tissue and the PMP22 protein is largely produced within the Schwann cells within this region. Thus, in order to effective measure the effect of any drug on the Schwann cells, a major tissue biopsy must be performed and/or the animal must be sacrificed in order to assess these tissues. These measurements are not practical or possible in human patients during drug clinical trials or treatment. Thus, measuring the activity over time is not possible in humans and difficult in animals. Often, for laboratory experiments, large groups of animals will be studied and periodically sacrificed to assess these tissues.

While the majority of the pre-mRNA and mRNA for PMP22 is located within the cells and tissue of interest, a very small, but detectable amount of both materials will be present in the blood stream or other biological fluids that are accessible without major biopsy or autopsy. This material in the blood (while not synthesized there), will be present from cells within the body dying and RNA leaking out into the blood stream non-specifically. Other materials may be present from damaged cells or non-specific cellular expulsion. For previously described drug approaches where the total mRNA is reduced as the mode of action of the drug, it is impractical to measure a slight lowering of total mRNA in the blood stream from the effect of the drug in a large background variance due to natural fluctuations (from exercise, food, metabolism, disease, damage, etc.). These variance in total natural mRNA can be quite large over even a 24 hour period, thus quantitating a small decline due to the drug is impractical. In certain embodiments, there will be nearly zero amount of exon-skipped PMP22 in the blood stream without the presence of an exon-skipping drug. Thus, if the amount of exon-skipped mRNA is quantitated in the blood stream, it will be directly proportional to the activity of the drug. Even more quantitative will be to compare the ratio of the amount of full-length PMP22 mRNA to exon-skipped mRNA within the blood stream at any given time to confirm and quantitate the drug activity. For example, a day after a drug is given to a patient, the drug has had sufficient time to be effectively distributed throughout the body and into the tissues and cells of interest. A near maximal amount of new PMP22 protein production at the cellular level should be achieved for a given drug composition and dose at this time. The concentration of exon-skipped mRNA in the blood than can be periodically monitored from the blood over time and correlated to drug activity. Even more preferrable would be to quantitate both the full-length and exon-skipped mRNA and correlate that ratio to drug activity. This method gives an internal control to the measurement which simplifies that assay measurement and increases accuracy. As that value goes down over time (indicative of new cells being created that do not contain the drug, loss of activity of the drug, etc.), an additional dose of the drug chosen can be given at that time to re-equilibrated exon skipping to the desired value.

The method of this disclosure of measuring the exon-skipped mRNA in the blood can also be used to adjust the initial dose on a patient-by-patient basis or during dose escalation studies during clinical trials. For CMT 1A (and other diseases) the variance in actual measurements (versus true molecular biology activity) could be disastrous for certain patients. Incorrect results could lead to underdosing of patients (thus missing the drug activity window) or significant overdosing of patients (leading to potentially dangerous side effects). Due to a number of factors, two different patients may require different amounts of drug to achieve desired levels of PMP22 reduction. For instance, if it is desirable to block 50% of PMP22 protein production, a physically smaller patient may require less drug than a heavier patient. Patients with different metabolisms or lifestyles may also require different amounts of drug per treatment to achiever optimal molecular changes. By producing a stable mRNA product within the animal or patient that is not naturally occurring (as described in this disclosure), these measurement are made possible.

### Example 8

A key consideration for the treatment of CMT is to determine how the treatment may be effective for older patients that have progressed in the disease for a longer period of time and may have more axon damage than other patients. C3 animals were allowed to progress until 3 months of age and monitored for their walking ability across a suspended dowel as described above. The amount of time to cross the dowel was monitored prior to their treatment. The animals where then treated with 17 mg/kg of SCH-012 weekly for 6 weeks then allowed to progress (untreated) for an additional 6 weeks and again monitored for walking capability.

FIG. 21 shows the results of this experiment (with 3 month old wild-type animals also plotted for comparison). As can be seen, the group of animals were performing very poorly at 3 months of age (average dowel travers time of 18.1 seconds with a standard deviation of 7.1 seconds). After treatment and a recovery period, the same animals significantly improved to and average walking time of 7.1 seconds with a standard deviation of 2.4 seconds). For each bar in the graph, animals were measured for 4 days (2 days apart) and the averages of those time points are shown.

### Example 9

Another group of C3 animals were allowed to progress untreated until 12 months of age. At 12 months of age, wild-type animals will also begin to show some degradation in walking and balance ability, so wild-type animals were also studied. At 12 months of age, C3 animals were treated (Table 1). For treatment groups 1, 2, 3, 5, 6, and 7, five animals were studied in each group and for groups 4 and 5, four animals were studied.

TABLE 1

| | Animal | | | Testing | Testing | Testing | |
|---|---|---|---|---|---|---|---|
| GROUP | Type | Treatment | Treatment Days | Time 1 | Time 2 | Time 3 | Sacrifice |
| 1 | C3 | SHC1A-012 50 mg/kg | Day 1 | Day 0 | Day 8 | | Day 8 |
| 2 | C3 | SHC1A-012 50 mg/kg | Day 1 | Day 0 | Day 30 | | Day 30 |
| 3 | C3 | SHC1A-012 50 mg/kg | Day 1 | Day 0 | | Day 60 | Day 60 |
| 4 | C3 | SHC1A-012 50 mg/kg - 2 | Day 1, Day 31 | Day 0 | Day 30 | Day 60 | Day 60 |
| 5 | C3 | SHC1A-012 5 mg/kg - 2 | Day 1, Day 31 | Day 0 | Day 30 | Day 60 | Day 60 |
| 6 | C3 | 50 mg/kg Scramble | Day 1 | Day 0 | | | |
| 7 | WT | None | None | Day 0 | | | |

The raw performance results of the testing are shown in Table 2. For the dowl walking time, the same apparatus was used as before, and the times (in seconds) were recorded per animal two days in a row and the average for the group calculated using both days. For grip strength, each data set is the front paw grip strength (in grams for a 25 Newton pull setting) for the entire group tested on one day.

TABLE 2

| | | Dowel Walking Time | | | Grip Strength | | |
|---|---|---|---|---|---|---|---|
| GROUP | Treatment | Time 1 | Time 2 | Time 3 | Time 1 | Time 2 | Time 3 |
| 1 | SHC1A-012 50 mg/kg | 14.2 | 15.8 | | 53 | 54 | |
| 2 | SHC1A-012 50 mg/kg | 15.1 | 11.2 | | 38 | 58 | |
| 3 | SHC1A-012 50 mg/kg | 13.4 | 11.6 | | 40 | 56 | |
| 4 | SHC1A-012 50 mg/kg - 2 | 16.1 | 10.8 | 7.3 | 20 | 38 | 72 |
| 5 | SHC1A-012 5 mg/kg - 2 | 14.3 | 13.9 | 8.5 | 40 | 44 | 44 |
| 6 | 50 mg/kg Scramble | 16.5 | 8.8 | | 43 | 43 | |
| 7 | None | 7.2 | 6.5 | | 99 | 92 | |

Table 3 shows the data from Table 2 where the average percent improvement (compared to the first day for each group separately) is shown. For example, for Group 2 dowel walking time, the animals in the group showed on average a 26% improvement in walking performance from before treatment and after treatment (since a reduction in time is an improvement for walking). For the animals in groups 4 and 5 that had two performance testing days (30 days and 60 days), both time point percent improvements are compared to the original pre-testing data. For example, for Group 4 grip strength, the animals showed a 90% improvement after 1 treatment (Time 1-2) and a 256% improvement at the 60 day mark compared to the pre-treatment value. Positive increases in grip strength demonstrate improvement.

TABLE 3

| | Percent Improvement | Dowel Walking Time | | Grip Strength | |
|---|---|---|---|---|---|
| GROUP | Treatment | Time 1-2 | Time 2-3 | Time 1-2 | Time 2-3 |
| 1 | SHC1A-012 50 mg/kg | 11% | | 3% | |
| 2 | SHC1A-012 50 mg/kg | −26% | | 55% | |
| 3 | SHC1A-012 50 mg/kg | −13% | | 39% | |
| 4 | SHC1A-012 50 mg/kg - 2 | −33% | −55% | 90% | 256% |
| 5 | SHC1A-012 5 mg/kg - 2 | −3% | −40% | 10% | 11% |
| 6 | 50 mg/kg Scramble | −46% | | 0% | |
| 7 | None | −10% | | −7% | |

After the sacrifice times shown in Table 1 in this example, nerves were extracted for quantitative PCR analysis to determine the total amount of human PMP22 mRNA present in the nerves. Effective SHC-012 performance should lower the amount of human PMP22 mRNA as compared to the scramble treated animals. The data from all animals is shown in Table 4. For percent reduction, the amount of PMP22 mRNA in the scramble group was determined and the percent reduction of each treatment group (compared to that scramble control) was calculated.

TABLE 4
Human PMP22% Reduction

| GROUP | Treatment | Sacrifice Time | | |
|---|---|---|---|---|
| 1 | SHC1A-012 50 mg/kg | 1 | week | 61% |
| 2 | SHC1A-012 50 mg/kg | 2 | Months | 96% |
| 3 | SHC1A-012 50 mg/kg | 1 | Month | 96% |
| 4 | SHC1A-012 50 mg/kg - 2 | 2 | Months | 86% |
| 5 | SHC1A-012 5 mg/kg - 2 | 2 | Months | 95% |

For the performance data shown above, wild-type animals were also monitored to assess whether the different treatment days had any effect on the values. The numbers in Table 3 demonstrate a small effect which is likely simply due to testing variance (10% improvement in walking time but 7% detrimental results in grip strength).

Certain embodiments of the present disclosure can be defined in any of the following numbered paragraphs:
1. A composition comprising an antisense oligonucleotide (ASO), wherein the ASO comprises or consists of a complementary region that is complementary, or complementary except for one, two, three, four, or five mismatched nucleotides, to a target region of a PMP22 pre-mRNA;
wherein binding in a cell of the complementary region of the ASO to the target region of the PMP22 pre-mRNA induces exon skipping during RNA transcription, thereby reducing full-length PMP22 mRNA production and producing exon-skipped PMP22 mRNA.
2. The composition of paragraph 1,
wherein the ASO comprises or consists of a complementary region of at least about 12, 13, 14, 15, 16, 17, 18, 19, 20, 21, 22, 23, or 24 contiguous nucleotides that are complementary, or complementary except for one, two, three, four, or five mismatched nucleotides, to the PMP22 pre-mRNA target region; or
wherein the PMP22 pre-mRNA target region comprises two separate segments of the PMP22 pre-mRNA, and optionally, wherein the ASO comprises or consists of a complementary region of at least about 6, 8, 9, 10, 11, or 12 nucleotides that are complementary, or complementary except for one, two, or three mismatched nucleotides, to a first segment of contiguous sequence of the PMP22 pre-mRNA target region and wherein the ASO comprises or consists of a complementary region of at least about 6, 8, 9, 10, 11, or 12 nucleotides that are complementary, or complementary except for one, two, or three mismatched nucleotides, to a second segment of contiguous sequence of the PMP22 pre-mRNA target region.
3. The composition of paragraph 1 or 2, wherein the ASO comprises or consists of a complementary region between any of about 12, 13, 14, 15, 16, 17, 18, 19, 20, 21, 22, 23, 24, 25, 30, 35, 40, or 45 and any of about 13, 14, 15, 16, 17, 18, 19, 20, 21, 22, 23, 24, 25, 26, 27, 28, 29, 30, 35, 40, 45, or 50 nucleotides that are complementary, or complementary except for one, two, three, four, or five mismatched nucleotides, to the PMP22 pre-mRNA target region;
optionally, wherein the ASO comprises or consists of a complementary region of 12, 13, 14, 15, 16, 17, 18, 19, 20, 21, 22, 23, 24, or 25 nucleotides that are complementary, or complementary except for one, two, three, four, or five mismatched nucleotides, to the PMP22 pre-mRNA target region.
4. The composition of any one of paragraphs 1 to 3, wherein the ASO is a modified and/or synthetic oligonucleotide;
optionally, wherein the ASO is a phosphorodiamidate morpholino oligomer (PMO).
5. The composition of any one of paragraphs 1 to 4, wherein downstream exons are still expressed.
6. The composition of any one of paragraphs 1 to 4, wherein the exon skipping forces downstream exons to be out of frame.
7. The composition of any one of paragraphs 1 to 6, wherein the target region of the PMP22 pre-mRNA spans an intron/exon junction of one of the coding exons;
optionally, wherein the exon portion of the intron/exon junction comprises PMP22 Exon 3; and/or
optionally, wherein the exon portion of the intron/exon junction comprises PMP22 Exon 4.
8. The composition of any one of paragraphs 1 to 7, wherein the target region of the PMP22 pre-mRNA comprises the 5′-end of an exon.
9. The composition of any one of paragraphs 1 to 7, wherein the target region of the PMP22 pre-mRNA comprises the 3′-end of an exon.
10. The composition of any one of paragraphs 7 to 9, wherein the target region of the PMP22 pre-mRNA spans an intron/exon junction comprising or consisting of at least 2, 3, 4, 5, 6, 7, 8, 9, 10, 11, or 12 nucleotides of the intron and a portion of the exon;
optionally, where the target region of the PMP22 pre-mRNA consists of 2, 3, 4, 5, 6, 7, 8, 9, 10, 11, or 12 nucleotides of the intron and a portion of the exon.
11. The composition of any one of paragraphs 7 to 10, wherein the target region of the PMP22 pre-mRNA spans an intron/exon junction comprising or consisting of at least 2, 3, 4, 5, 6, 7, 8, 9, 10, 11, or 12 nucleotides of the exon and a portion of the intron;
optionally, where the target region of the PMP22 pre-mRNA consists of 2, 3, 4, 5, 6, 7, 8, 9, 10, 11, or 12 nucleotides of the exon and a portion of the intron.
12. The composition of any one of paragraphs 1 to 11, wherein the PMP22 pre-mRNA target region comprises or consists of SEQ ID NO: 2 (Exon 3, 5′-end), SEQ ID NO: 35 (Exon 3, 3′-end), SEQ ID NO: 76 (Exon 4, 5′-end), SEQ ID NO: 111 (Exon 4, 3′-end), SEQ ID NO: 163 (Exon 2, 5′-end), and/or SEQ ID NO: 198 (Exon 2, 3′-end), or a portion or subset/fragment thereof.
13. The composition of paragraph 12, wherein the ASO comprises or consists of a complementary region that is complementary, or complementary except for one, two, three, four, or five mismatched nucleotides, to SEQ ID NO: 2 (Exon 3, 5′-end), SEQ ID NO: 35 (Exon 3, 3′-end), SEQ ID NO: 76 (Exon 4, 5′-end), SEQ ID NO: 111 (Exon 4, 3′-end), SEQ ID NO: 163 (Exon 2, 5′-end), and/or SEQ ID NO: 198 (Exon 2, 3′-end);
optionally, wherein the ASO comprises or consists of a nucleotide sequence of SEQ ID NOs: 3-34 (Exon 3, 5′-end), SEQ ID NOs: 37-70 (Exon 3, 3′-end), SEQ ID NOs: 77-110 (Exon 4, 5′-end), SEQ ID NOs: 112-145 (Exon 4, 3′-end), SEQ ID NOs: 164-197 (Exon 2, 5′-end), or SEQ ID NOs: 199-232 (Exon 2, 3′-end), or fragment thereof sufficient to hybridize to PMP22 pre-mRNA; or
wherein the ASO comprises or consists of a nucleotide sequence of SEQ ID NOs: 3-34 (Exon 3, 5′-end), SEQ ID NOs: 37-70 (Exon 3, 3′-end), SEQ ID NOs: 77-110 (Exon 4, 5′-end), SEQ ID NOs: 112-145 (Exon 4, 3′-end), SEQ ID NOs: 164-197 (Exon 2, 5′-end), or SEQ ID NOs: 199-232 (Exon 2, 3′-end), except for having one, two, or three nucleotide substitutions, or fragment thereof sufficient to hybridize to PMP22 pre-mRNA.
14. The composition of paragraph 1:
   wherein the ASO comprises or consists of the nucleic acid sequence of:
SEQ ID NO: 71 (SHC-006 25-mer),
SEQ ID NO: 72 (SHC-001 24-mer),
SEQ ID NO: 73 (SHC-005 25-mer),
SEQ ID NO: 74 (SHC-010 21-mer),
SEQ ID NO: 75 (SHC-012 20-mer),
SEQ ID NO: 146 (SHC-029 21-mer),
SEQ ID NO: 147 (SHC-028 20-mer),
SEQ ID NO: 148 (SHC-027 20-mer),
SEQ ID NO: 149 (SHC-031 21-mer),
SEQ ID NO: 150 (SHC-030 20-mer),
SEQ ID NO: 151 (SHC-032 20-mer),
SEQ ID NO: 156,
SEQ ID NO: 159,
SEQ ID NO: 162,
SEQ ID NO: 235, or
SEQ ID NO: 238;
   or wherein the ASO comprises or consists of the nucleic acid sequence of:
SEQ ID NO: 71 (SHC-006 25-mer),
SEQ ID NO: 72 (SHC-001 24-mer),
SEQ ID NO: 73 (SHC-005 25-mer),
SEQ ID NO: 74 (SHC-010 21-mer),
SEQ ID NO: 75 (SHC-012 20-mer),
SEQ ID NO: 146 (SHC-029 21-mer),
SEQ ID NO: 147 (SHC-028 20-mer),
SEQ ID NO: 148 (SHC-027 20-mer),
SEQ ID NO: 149 (SHC-031 21-mer),
SEQ ID NO: 150 (SHC-030 20-mer),
SEQ ID NO: 151 (SHC-032 20-mer),
SEQ ID NO: 156,
SEQ ID NO: 159,
SEQ ID NO: 162,
SEQ ID NO: 235, or
SEQ ID NO: 238,
   except for having one, two, or three nucleotide substitutions.
15. A method of decreasing the amount of full-length PMP22 mRNA expression in a cell, the method comprising administering to the cell a composition comprising an antisense oligonucleotide (ASO) of any one of paragraphs 1 to 14;
optionally, wherein an PMP22 exon-skipped mRNA is produced; and/or
optionally, wherein the amount of functional PMP22 protein produced in the cell is decreased.
16. The method of paragraph 15,
wherein the amount of full-length PMP22 mRNA in the cell is decreased by at least about 5%, 10%, 15%, 20%, 25%, 30%, 40%, 50%, 60%, 75%, or 95% in response to the ASO,
and/or
wherein the amount of PMP22 mRNA in the cell is decreased by not more than about 25%, 30%, 40%, 50%, 60%, 70%, 80%, 90%, or 95% in response to the ASO.
17. The method of paragraph 15 or 16, wherein the amount of full-length PMP22 mRNA in the cell is decreased by from any of about 5%, 10%, 15%, 20%, 25%, 30%, 40%, 50%, 60%, or 75% to any of about 25%, 30%, 40%, 50%, 60%, 70%, 80%, 90%, or 95% in response to the ASO.
18. The method of any one of paragraphs 15 to 17,
wherein the amount of functional PMP22 protein in the cell is decreased by at least about 5%, 10%, 15%, 20%, 25%, 30%, 40%, 50%, 60%, 75%, or 95% in response to the ASO,
and/or
wherein the amount of functional PMP22 protein in the cell is decreased by not more than about 25%, 30%, 40%, 50%, 60%, 70%, 80%, 90%, or 95% in response to the ASO.
19. The method of paragraph 17 or 18, wherein the amount of functional PMP22 protein in the cell is decreased by from any of about 5%, 10%, 15%, 20%, 25%, 30%, 40%, 50%, 60%, or 75% to any of about 25%, 30%, 40%, 50%, 60%, 70%, 80%, 90%, or 95% in response to the ASO.
20. The method of any one of paragraphs 15 to 19, wherein the method comprises targeting a junction between an intron and an exon within a PMP22 pre-mRNA.
21. A method of producing an exon-skipped PMP22 pre-mRNA, the method comprising administering to a cell a composition comprising an antisense oligonucleotide (ASO) of any one of paragraphs 1 to 14.
22. The method of paragraph 21, wherein the amount of full-length PMP22 mRNA expression in the cell is decreased;
optionally, wherein the amount of functional PMP22 protein produced in the cell is decreased.
23. The method of paragraph 21 or 22, wherein the method comprises targeting a junction between an intron and an exon within a PMP22 pre-mRNA.
24. A method of treating Charcot-Marie-Tooth disease, the method comprising administering to a subject in need thereof a composition comprising an antisense oligonucleotide (ASO) of any one of paragraphs 1 to 14.
25. The method of paragraph 24, wherein the ASO is administered as a pharmaceutically acceptable salt.
26. The method of paragraph 24 or 25, wherein the ASO is administered in a pharmaceutically acceptable carrier or diluent.
27. The method of any one of paragraphs 24 to 26, wherein at least one symptom of the disease is alleviated.
28. The method of any one of paragraphs 24 to 26, wherein the rate of progression of at least one symptom of the disease is decreased.
29. The method of any one of paragraphs 24 to 28, wherein the dosing regimen for administering the composition is based on the age of the subject.
30. The method of any one of paragraphs 24 to 29, wherein the dosing regimen for administering the composition is based on the symptom progression of the subject.
31. The method of any one of paragraphs 24 to 30, wherein the dosing regimen for administering the composition is based on the overall body weight of the subject.
32. The method of any one of paragraphs 24 to 31, wherein the dosing regimen for administering the composition is based on the physical performance of the subject.
33. The method of any one of paragraphs 24 to 32, wherein a subject is given a higher dose or a loading dose of the composition based on greater symptom severity and/or older age for a period of time, which is later changed to a lower does.
34. A composition comprising an antisense oligonucleotide (ASO), wherein the ASO comprises or consists of a complementary region that is complementary, or complementary except for one, two, three, four, or five mismatched nucleotides, to a target region of a PMP22 pre-mRNA;
wherein the target region of the PMP22 pre-mRNA comprises an intron/exon junction of one of the coding exons.
35. The composition of paragraph 34,
wherein the ASO comprises or consists of a complementary region of at least about 12, 13, 14, 15, 16, 17, 18, 19, 20, 21, 22, 23, or 24 contiguous nucleotides that are complementary, or complementary except for one, two, three, four, or five mismatched nucleotides, to the PMP22 pre-mRNA target region; or
wherein the PMP22 pre-mRNA target region comprises two separate segments of the PMP22 pre-mRNA, and optionally, wherein the ASO comprises or consists of a complementary region of at least about 6, 8, 9, 10, 11, or 12 nucleotides that are complementary, or complementary except for one, two, or three mismatched nucleotides, to a first segment of contiguous sequence of the PMP22 pre-mRNA target region and wherein the ASO comprises or consists of a complementary region of at least about 6, 8, 9, 10, 11, or 12 nucleotides that are complementary, or complementary except for one, two, or three mismatched nucleotides, to a second segment of contiguous sequence of the PMP22 pre-mRNA target region.
36. The composition of paragraph 34 or 35, wherein the ASO comprises or consists of a complementary region between any of about 12, 13, 14, 15, 16, 17, 18, 19, 20, 21, 22, 23, 24, 25, 30, 35, 40, or 45 and any of about 13, 14, 15, 16, 17, 18, 19, 20, 21, 22, 23, 24, 25, 26, 27, 28, 29, 30, 35, 40, 45, or 50 contiguous nucleotides that are complementary, or complementary except for one, two, three, four, or five mismatched nucleotides, to the PMP22 pre-mRNA target region;
optionally, wherein the ASO comprises or consists of a complementary region of 16, 17, 18, 19, 20, 21, 22, 23, 24, or 25 contiguous nucleotides that are complementary, or complementary except for one, two, three, four, or five mismatched nucleotides, to the PMP22 pre-mRNA target region.
37. The composition of any one of paragraphs 34 to 36,
wherein the exon portion of the intron/exon junction comprises PMP22 Exon 3; and/or
wherein the exon portion of the intron/exon junction comprises PMP22 Exon 4.
38. The composition of any one of paragraphs 34 to 37, wherein the target region of the PMP22 pre-mRNA comprises the 3′-end of an exon.
39. The composition of any one of paragraphs 34 to 37, wherein the target region of the PMP22 pre-mRNA comprises the 5′-end of an exon.
40. The composition of any one of paragraphs 34 to 39, wherein the target region of the PMP22 pre-mRNA comprises an intron/exon junction comprising at least 2, 3, 4, 5, 6, 7, 8, 9, 10, 11, or 12 nucleotides of the intron and a portion of the exon;
optionally, where the target region of the PMP22 pre-mRNA comprises or consists of 2, 3, 4, 5, 6, 7, 8, 9, 10, 11, or 12 nucleotides of the intron and a portion of the exon.
41. The composition of any one of paragraphs 34 to 40, wherein the target region of the PMP22 pre-mRNA comprises an intron/exon junction comprising at least 2, 3, 4, 5, 6, 7, 8, 9, 10, 11, or 12 nucleotides of the exon and a portion of the intron;
optionally, where the target region of the PMP22 pre-mRNA comprises or consists of 2, 3, 4, 5, 6, 7, 8, 9, 10, 11, or 12 nucleotides of the exon and a portion of the intron.
42. The composition of any one of paragraphs 34 to 41, wherein ASO is a modified and/or synthetic oligonucleotide;
optionally, wherein the ASO is a phosphorodiamidate morpholino oligomer (PMO).
43. The composition of any one of paragraphs 34 to 42, wherein the PMP22 pre-mRNA target region comprises or consists of SEQ ID NO: 2 (Exon 3, 5′-end), SEQ ID NO: 35 (Exon 3, 3′-end), SEQ ID NO: 76 (Exon 4, 5′-end), SEQ ID NO: 111 (Exon 4, 3′-end), SEQ ID NO: 163 (Exon 2, 5′-end), and/or SEQ ID NO: 198 (Exon 2, 3′-end).

The breadth and scope of the present disclosure should not be limited by any of the above-described exemplary embodiments, but should be defined only in accordance with the following claims and their equivalents.

### REFERENCES

Krajewski K M, Lewis R A, Fuerst D R, Turansky C, Hinderer S R, Garbern J, Kamholz J, Shy M E. Neurological dysfunction and axonal degeneration in Charcot-Marie-Tooth disease type 1A. Brain: a journal of neurology. 2000; 123 (Pt 7):1516-27. Epub 2000/06/27. doi: 10.1093/brain/123.7.1516. PubMed PMID: 10869062.
Braathen G J. Genetic epidemiology of Charcot-Marie-Tooth disease. Acta Neurol Scand Suppl. 2012(193):iv-22. Epub 2012/11/01. doi: 10.1111/ane.12013. PubMed PMID: 23106488.
Patel P I, Roa B B, Welcher A A, Schoener-Scott R, Trask B J, Pentao L, Snipes G J, Garcia C A, Francke U, Shooter E M, Lupski J R, Suter U. The gene for the peripheral myelin protein PMP-22 is a candidate for Charcot-Marie-Tooth disease type 1A. Nature genetics. 1992; 1(3):159-65. Epub 1992/06/01. doi: 10.1038/ng0692-159. PubMed PMID: 1303228.
Timmerman V, Nelis E, Van Hul W, Nieuwenhuijsen B W, Chen K L, Wang S, Ben Othman K, Cullen B, Leach R J, Hanemann C O, et al. The peripheral myelin protein gene PMP-22 is contained within the Charcot-Marie-Tooth disease type 1A duplication. Nature genetics. 1992; 1(3):171-5. Epub 1992/06/01. doi: 10.1038/ng0692-171. PubMed PMID: 1303230.
Valentijn L J, Bolhuis P A, Zorn I, Hoogendijk J E, van den Bosch N, Hensels G W, Stanton V P, Jr., Housman D E, Fischbeck K H, Ross D A, et al. The peripheral myelin gene PMP-22/GAS-3 is duplicated in Charcot-Marie-Tooth disease type 1A. Nature genetics. 1992; 1(3):166-70. Epub 1992/06/11. doi: 10.1038/ng0692-166. PubMed PMID: 1303229.
Matsunami N, Smith B, Ballard L, Lensch M W, Robertson M, Albertsen H, Hanemann C O, Muller H W, Bird T D, White R, et al. Peripheral myelin protein-22 gene maps in the duplication in chromosome 17p11.2 associated with Charcot-Marie-Tooth 1A. Nature genetics. 1992; 1(3):176-9. Epub 1992/06/01. doi: 10.1038/ng0692-176. PubMed PMID: 1303231.
Lee S, Amici S, Tavori H, Zeng W M, Freeland S, Fazio S, Notterpek L. PMP22 is critical for actin-mediated cellular functions and for establishing lipid rafts. The Journal of neuroscience: the official journal of the Society for Neuroscience. 2014; 34(48):16140-52. Epub 2014/11/28. doi: 10.1523/JNEUROSCI.1908-14.2014. PubMed PMID: 25429154; PMCID: PMC4244477.
Mittendorf K F, Marinko J T, Hampton C M, Ke Z, Hadziselimovic A, Schlebach J P, Law C L, Li J, Wright E R, Sanders C R, Ohi M D. Peripheral myelin protein 22 alters membrane architecture. Sci Adv. 2017; 3(7):e1700220. Epub 2017/07/12. doi: 10.1126/sciadv.1700220. PubMed PMID: 28695207; PMCID: PMC5498104.
Snipes G J, Suter U, Welcher A A, Shooter E M. Characterization of a novel peripheral nervous system myelin protein (PMP-22/SR13). The Journal of cell biology. 1992; 117(1):225-38. Epub 1992/04/01. doi: 10.1083/jcb.117.1.225. PubMed PMID: 1556154; PMCID: PMC2289391.
Magyar J P, Martini R, Ruelicke T, Aguzzi A, Adlkofer K, Dembic Z, Zielasek J, Toyka K V, Suter U. Impaired differentiation of Schwann cells in transgenic mice with increased PMP22 gene dosage. The Journal of neuroscience: the official journal of the Society for Neuroscience. 1996; 16(17):5351-60. Epub 1996/09/01. PubMed PMID: 8757248; PMCID: PMC6578876.
Sereda M, Griffiths I, Puhlhofer A, Stewart H, Rossner M J, Zimmerman F, Magyar J P, Schneider A, Hund E, Meinck H M, Suter U, Nave K A. A transgenic rat model of Charcot-Marie-Tooth disease. Neuron. 1996; 16(5):1049-60. Epub 1996/05/01. doi: 10.1016/s0896-6273(00)80128-2. PubMed PMID: 8630243.
Huxley C, Passage E, Manson A, Putzu G, Figarella-Branger D, Pellissier J F, Fontes M. Construction of a mouse model of Charcot-Marie-Tooth disease type 1A by pronuclear injection of human YAC DNA. Human molecular genetics. 1996; 5(5):563-9. Epub 1996/05/01. doi: 10.1093/hmg/5.5.563. PubMed PMID: 8733121.
Perea J, Robertson A, Tolmachova T, Muddle J, King R H, Ponsford S, Thomas P K, Huxley C. Induced myelination and demyelination in a conditional mouse model of Charcot-Marie-Tooth disease type 1A. Human molecular genetics. 2001; 10(10):1007-18. Epub 2001/05/02. doi: 10.1093/hmg/10.10.1007. PubMed PMID: 11331611.
Chance P F, Alderson M K, Leppig K A, Lensch M W, Matsunami N, Smith B, Swanson P D, Odelberg S J, Disteche C M, Bird T D. DNA deletion associated with hereditary neuropathy with liability to pressure palsies. Cell. 1993; 72(1):143-51. Epub 1993/01/15. doi: 10.1016/0092-8674(93)90058-x. PubMed PMID: 8422677.
Zhao H T, Damle S, Ikeda-Lee K, Kuntz S, Li J, Mohan A, Kim A, Hung G, Scheideler M A, Scherer S S, Svaren J, Swayze E E, Kordasiewicz H B. PMP22 antisense oligonucleotides reverse Charcot-Marie-Tooth disease type 1A features in rodent models. The Journal of clinical investigation. 2018; 128(1):359-68. Epub 2017/12/05. doi: 10.1172/JC196499. PubMed PMID: 29202483; PMCID: PMC5749515.
Boutary, Suzan et al. “Squalenoyl siRNA PMP22 nanoparticles are effective in treating mouse models of Charcot-Marie-Tooth disease type 1 A.” Communications biology vol. 4,1 317. 9 Mar. 2021, doi:10.1038/s42003-021-01839-2.
Cornett, Kayla M D et al. “Natural history of Charcot-Marie-Tooth disease during childhood.” Annals of neurology vol. 82,3 (2017): 353-359. doi:10.1002/ana.25009 18.Cortese, Andrea et al. “Targeted next-generation sequencing panels in the diagnosis of Charcot-Marie-Tooth disease.” Neurology vol. 94,1 (2020): e51-e61. doi:10.1212/WNL.0000000000008672.
Eichinger, Katy et al. “The Charcot-Marie-Tooth Functional Outcome Measure (CMT FOM).” Neurology vol. 91,15 (2018): e1381-e1384. doi:10.1212/WNL.0000000000006323.
Gautier, Benoit et al. “AAV2/9-mediated silencing of PMP22 prevents the development of pathological features in a rat model of Charcot-Marie-Tooth disease 1 A.” Nature communications vol. 12,1 2356. 21 Apr. 2021, doi:10.1038/s41467-021-22593-3 21. Kagiava, A et al. “Intrathecal gene therapy in mouse models expressing CMT1X mutations.” Human molecular genetics vol. 27,8 (2018): 1460-1473. doi:10.1093/hmg/ddy056.
Lee, Ji-Su et al. “Targeted PMP22 TATA-box editing by CRISPR/Cas9 reduces demyelinating neuropathy of Charcot-Marie-Tooth disease type 1A in mice.” Nucleic acids research vol. 48,1 (2020): 130-140. doi:10.1093/nar/gkz1070.
Massade, Liliane, and Charbel Massaad. “Antisense RNA Targeting PMP22 for the Treatment of Charcot Marie-Tooth 1A Disease.” EPO Patent 18306241.3. 1 Apr. 2020.
Morelli, Kathryn H et al. “Allele-specific RNA interference prevents neuropathy in Charcot-Marie-Tooth disease type 2D mouse models.” The Journal of clinical investigation vol. 129,12 (2019): 5568-5583. doi:10.1172/JC1130600.
Reynolds, Angela et al. “Rational siRNA design for RNA interference.” Nature biotechnology vol. 22,3 (2004): 326-30. doi:10.1038/nbt936 26. Sandelius, Asa et al. “Plasma neurofilament light chain concentration in the inherited\peripheral neuropathies.” Neurology vol. 90,6 (2018): e518-e524. doi:10.1212/WNL.0000000000004932.
Schorling, Elisabeth et al. “Cost of illness in Charcot-Marie-Tooth neuropathy: Results from Germany.” Neurology vol. 92,17 (2019): e2027-e2037. doi:10.1212/WNL.0000000000007376
Stavrou, Marina et al. “Emerging Therapies for Charcot-Marie-Tooth Inherited Neuropathies.” International journal of molecular sciences vol. 22,11 6048. 3 Jun. 2021, doi:10.3390/ijms22116048.
Stavrou, Marina et al. “Genetic mechanisms of peripheral nerve disease.” Neuroscience letters vol. 742 (2021): 135357. doi:10.1016/j.neulet.2020.135357.
Tozza, S et al. “Motor performance deterioration accelerates after 50 years of age in Charcot-Marie-Tooth type 1A patients.” European journal of neurology vol. 25,2 (2018): 301-306. doi:10.111 1/ene.13494.
Wang, Hongge et al. “MicroRNAs as Biomarkers of Charcot-Marie-Tooth Disease Type 1A.” Neurology vol. 97,5 (2021): e489-e500. doi:10.1212/WNL.0000000000012266.
Wang, Hongge et al. “Transmembrane protease serine 5: a novel Schwann cell plasma marker for CMT1A.” Annals of clinical and translational neurology vol. 7,1 (2020):69-82. doi:10.1002/acn3.50965.
Passage E, Norreel J C, Noack-Fraissignes P, Sanguedolce V, Pizant J, Thirion X, Robaglia-Schlupp A, Pellissier J F, Fontes M. Ascorbic acid treatment corrects the phenotype of a mouse model of Charcot-Marie-Tooth disease. Nat Med. 2004; 10(4):396-401. doi: 10.1038/nm1023. PubMed PMID: 15034573.
Gess B, Baets J, De Jonghe P, Reilly M M, Pareyson D, Young P. Ascorbic acid for the treatment of Charcot-Marie-Tooth disease. Cochrane Database Syst Rev. 2015(12):CD011952. Epub 2015/12/15. doi: 10.1002/14651858.CD011952. PubMed PMID: 26662471; PMCID: PMC6823270.
Pareyson D, Reilly M M, Schenone A, Fabrizi G M, Cavallaro T, Santoro L, Vita G, Quattrone A, Padua L, Gemignani F, Visioli F, Laura M, Radice D, Calabrese D, Hughes R A, Solari A, Cmt T, groups C-T. Ascorbic acid in Charcot-Marie-Tooth disease type 1A (CMT-TRIAAL and CMT-TRAUK): a double-blind randomised trial. Lancet Neurol. 2011; 10(4):320-8. doi: 10.1016/S1474-4422(11)70025-4. PubMed PMID: 21393063; PMCID: PMC3154498.
Pareyson D, Schenone A, Fabrizi G M, Santoro L, Padua L, Quattrone A, Vita G, Gemignani F, Visioli F, Solari A, Group C-T. A multicenter, randomized, double-blind, placebo-controlled trial of long-term ascorbic acid treatment in Charcot-Marie-Tooth disease type 1A (CMT-TRIAAL): the study protocol [EudraCT no.: 2006-000032-27]. Pharmacological research: the official journal of the Italian Pharmacological Society. 2006; 54(6):436-41. Epub 2006/10/13. doi: 10.1016/j.phrs.2006.09.001. PubMed PMID: 17029975.
Meyer zu Horste G, Prukop T, Liebetanz D, Mobius W, Nave K A, Sereda M W. Antiprogesterone therapy uncouples axonal loss from demyelination in a transgenic rat model of CMT1A neuropathy. Ann Neurol. 2007; 61(1):61-72. doi: 10.1002/ana.21026. PubMed PMID: 17262851.
Sereda M W, Meyer zu Horste G, Suter U, Uzma N, Nave K A. Therapeutic administration of progesterone antagonist in a model of Charcot-Marie-Tooth disease (CMT-1A). Nat Med. 2003; 9(12):1533-7. doi: 10.1038/nm957. PubMed PMID: 14608378.
Prukop T, Stenzel J, Wernick S, Kungl T, Mroczek M, Adam J, Ewers D, Nabirotchkin S, Nave K A, Hajj R, Cohen D, Sereda M W. Early short-term PXT3003 combinational therapy delays disease onset in a transgenic rat model of Charcot-Marie-Tooth disease 1A (CMT1A). PLoS One. 2019; 14(1):e0209752. doi: 10.1371/journal.pone.0209752. PubMed PMID: 30650121; PMCID: PMC6334894. [Pharnext, the employer of Serguei Nabirotchkin, Rodolphe Hajj and Daniel Cohen. DC, RH, MWS, TP and KAN submitted a patent based on this work: Full patent name: Early treatment of CMT disease Number: US2018/0000813. MWS, TP and KAN act as consultants to Pharnext. PXT3003 achieved a Phase 3 trial in CMT1A adult patients. There are no further patents related to this study, or marketed products to declare. This does not alter our adherence to all the PLOS ONE policies on sharing data and materials, as detailed online in the guide for authors.]
Sociali G, Visigalli D, Prukop T, Cervellini I, Mannino E, Venturi C, Bruzzone S, Sereda M W, Schenone A. Tolerability and efficacy study of P2X7 inhibition in experimental Charcot-Marie-Tooth type 1A (CMT1A) neuropathy. Neurobiol Dis. 2016; 95:145-57. doi: 10.1016/j.nbd.2016.07.017. PubMed PMID: 27431093.
Verhamme C, King R H, ten Asbroek A L, Muddle J R, Nourallah M, Wolterman R, Baas F, van Schaik I N. Myelin and axon pathology in a long-term study of PMP22-overexpressing mice. Journal of neuropathology and experimental neurology. 2011; 70(5):386-98. Epub 2011/04/14. doi: 10.1097/NEN.0b013e318217eba0. PubMed PMID: 21487305.
Landis S C, Amara S G, Asadullah K, Austin C P, Blumenstein R, Bradley E W, Crystal R G, Darnell R B, Ferrante R J, Fillit H, Finkelstein R, Fisher M, Gendelman H E, Golub R M, Goudreau J L, Gross R A, Gubitz A K, Hesterlee S E, Howells D W, Huguenard J, Kelner K, Koroshetz W, Krainc D, Lazic S E, Levine M S, Macleod M R, McCall J M, Moxley R T, 3rd, Narasimhan K, Noble L J, Perrin S, Porter J D, Steward 0, Unger E, Utz U, Silberberg S D. A call for transparent reporting to optimize the predictive value of preclinical research. Nature. 2012; 490(7419):187-91. Epub 2012/10/13. doi: 10.1038/naturel1556. PubMed PMID: 23060188; PMCID: 3511845.
FDA. Current Good Manufacturing Practice for Phase 1 Investigational Drugs. 2008.
FDA. CMC Regulatory Considerations for Oligonucleotide Drug Products: FDA Perspective. 2019.
FDA. IND Submissions for Individualized Antisense Oligonucleotide Drug Products: Administrative and Procedural Recommendations. 2020.
Fledrich R, Schlotter-Weigel B, Schnizer T J, Wichert S P, Stassart R M, Meyer zu Horste G, Klink A, Weiss B G, Haag U, Walter M C, Rautenstrauss B, Paulus W, Rossner M J, Sereda M W. A rat model of Charcot-Marie-Tooth disease 1A recapitulates disease variability and supplies biomarkers of axonal loss in patients. Brain: a journal of neurology. 2012; 135(Pt 1):72-87. Epub 2011/12/23. doi: 10.1093/brain/awr322. PubMed PMID: 22189569.
Gautier B, Hajjar H, Soares S, Berthelot J, Deck M, Abbou S, Campbell G, Ceprian M, Gonzalez S, Fovet C M, Schutza V, Jouvenel A, Rivat C, Zerah M, Francois V, Le Guiner C, Aubourg P, Fledrich R, Tricaud N. AAV2/9-mediated silencing of PMP22 prevents the development of pathological features in a rat model of Charcot-Marie-Tooth disease 1 A. Nature communications. 2021; 12(1):2356. Epub 2021/04/23. doi: 10.1038/s41467-021-22593-3. PubMed PMID: 33883545; PMCID: PMC8060274.
Duchesne M, Danigo A, Richard L, Vallat J M, Attarian S, Gonnaud P M, Lacour A, Pereon Y, Stojkovic T, Nave K A, Bertrand V, Nabirotchkin S, Cohen D, Demiot C, Magy L. Skin Biopsy Findings in Patients With CMT1A: Baseline Data From the CLN-PXT3003-01 Study Provide New Insights Into the Pathophysiology of the Disorder. Journal of neuropathology and experimental neurology. 2018; 77(4):274-81. doi: 10.1093/jnen/nly001. PubMed PMID: 29408953.
Svaren J, Moran J J, Wu X, Zuccarino R, Bacon C, Bai Y, Ramesh R, Gutmann L, Anderson D M, Pavelec D, Shy M E. Schwann cell transcript biomarkers for hereditary neuropathy skin biopsies. Annals of neurology. 2019; 85(6):887-98. Epub 20190422. doi: 10.1002/ana.25480. PubMed PMID: 30945774; PMCID: PMC7050273.
Wang H, Davison M, Wang K, Xia T H, Call K M, Luo J, Wu X, Zuccarino R, Bacha A, Bai Y, Gutmann L, Feely S M E, Grider T, Rossor A M, Reilly M M, Shy M E, Svaren J. MicroRNAs as Biomarkers of Charcot-Marie-Tooth Disease Type 1A. Neurology. 2021; 97(5):e489-e500. Epub 20210524. doi: 10.1212/WNL.0000000000012266. PubMed PMID: 34031204; PMCID: PMC8356381.
Park N Y, Kwak G, Doo H M, Kim H J, Jang S Y, Lee Y I, Choi B O, Hong Y B. Farnesol Ameliorates Demyelinating Phenotype in a Cellular and Animal Model of Charcot-Marie-Tooth Disease Type 1A. Curr Issues Mol Biol. 2021; 43(3):2011-21. Epub 20211113. doi: 10.3390/cimb43030138. PubMed PMID: 34889893.
Morrow J M, Evans M R B, Grider T, Sinclair C D J, Thedens D, Shah S, Yousry T A, Hanna M G, Nopoulos P, Thornton J S, Shy M E, Reilly M M. Validation of MRC Centre MRI calf muscle fat fraction protocol as an outcome measure in CMT1A. Neurology. 2018; 91(12):el125-e9. Epub 20180817. doi: 10.1212/WNL.0000000000006214. PubMed PMID: 30120135; PMCID: PMC6161551.
Scharner J, Qi S, Rigo F, Bennett C F, Krainer A R. Delivery of GalNAc-Conjugated Splice-Switching ASOs to Non-hepatic Cells through Ectopic Expression of Asialoglycoprotein Receptor. Mol Ther Nucleic Acids. 2019; 16:313-25. doi: 10.1016/j.omtn.2019.02.024. PubMed PMID: 30965276; PMCID: PMC6453860.
Brennan K M, Bai Y, Shy M E. Demyelinating CMT—what's known, what's new and what's in store?Neuroscience letters. 2015; 596:14-26. Epub 20150124. doi: 10.1016/j.neulet.2015.01.059. PubMed PMID: 25625223.
Morrow J M, Sinclair C D, Fischmann A, Machado P M, Reilly M M, Yousry T A, Thornton J S, Hanna M G. MRI biomarker assessment of neuromuscular disease progression: a prospective observational cohort study. Lancet neurology. 2016; 15(1):65-77. Epub 2015/11/10. doi: 10.1016/S1474-4422(15)00242-2. PubMed PMID: 26549782; PMCID: PMC4672173.
Fridman V, Sillau S, Acsadi G, Bacon C, Dooley K, Burns J, Day J, Feely S, Finkel R S, Grider T, Gutmann L, Herrmann D N, Kirk C A, Knause S A, Laura M, Lewis R A, Li J, Lloyd T E, Moroni I, Muntoni F, Pagliano E, Pisciotta C, Piscosquito G, Ramchandren S, Saporta M, Sadjadi R, Shy R R, Siskind C E, Sumner C J, Walk D, Wilcox J, Yum S W, Zuchner S, Scherer S S, Pareyson D, Reilly M M, Shy M E, Inherited Neuropathies Consortium-Rare Diseases Clinical Research N. A longitudinal study of CMT1A using Rasch analysis based CMT neuropathy and examination scores. Neurology. 2020; 94(9):e884-e96. Epub 2020/02/13. doi: 10.1212/WNL.0000000000009035. PubMed PMID: 32047073; PMCID: PMC7238948.

What is claimed is:

1. A composition comprising an antisense oligonucleotide (ASO), wherein the ASO comprises or consists of a complementary region that is complementary, or complementary except for one, two, three, four, or five mismatched nucleotides, to a target region of a PMP22 pre-mRNA;
wherein binding in a cell of the complementary region of the ASO to the target region of the PMP22 pre-mRNA induces exon skipping during RNA transcription, thereby reducing full-length PMP22 mRNA production and producing exon-skipped PMP22 mRNA.

2. The composition of claim 1,
wherein the ASO comprises or consists of a complementary region of at least about 12, 13, 14, 15, 16, 17, 18, 19, 20, 21, 22, 23, or 24 contiguous nucleotides that are complementary, or complementary except for one, two, three, four, or five mismatched nucleotides, to the PMP22 pre-mRNA target region; or
wherein the PMP22 pre-mRNA target region comprises two separate segments of the PMP22 pre-mRNA, and optionally, wherein the ASO comprises or consists of a complementary region of at least about 6, 8, 9, 10, 11, or 12 nucleotides that are complementary, or complementary except for one, two, or three mismatched nucleotides, to a first segment of contiguous sequence of the PMP22 pre-mRNA target region and wherein the ASO comprises or consists of a complementary region of at least about 6, 8, 9, 10, 11, or 12 nucleotides that are complementary, or complementary except for one, two, or three mismatched nucleotides, to a second segment of contiguous sequence of the PMP22 pre-mRNA target region.

3. The composition of claim 1 or 2, wherein the ASO comprises or consists of a complementary region between any of about 12, 13, 14, 15, 16, 17, 18, 19, 20, 21, 22, 23, 24, 25, 30, 35, 40, or 45 and any of about 13, 14, 15, 16, 17, 18, 19, 20, 21, 22, 23, 24, 25, 26, 27, 28, 29, 30, 35, 40, 45, or 50 nucleotides that are complementary, or complementary except for one, two, three, four, or five mismatched nucleotides, to the PMP22 pre-mRNA target region;
optionally, wherein the ASO comprises or consists of a complementary region of 12, 13, 14, 15, 16, 17, 18, 19, 20, 21, 22, 23, 24, or 25 nucleotides that are complementary, or complementary except for one, two, three, four, or five mismatched nucleotides, to the PMP22 pre-mRNA target region.

4. The composition of any one of claims 1 to 3, wherein the ASO is a modified and/or synthetic oligonucleotide;
optionally, wherein the ASO is a phosphorodiamidate morpholino oligomer (PMO).

5. The composition of any one of claims 1 to 4, wherein downstream exons are still expressed.

6. The composition of any one of claims 1 to 4, wherein the exon skipping forces downstream exons to be out of frame.

7. The composition of any one of claims 1 to 6, wherein the target region of the PMP22 pre-mRNA spans an intron/exon junction of one of the coding exons;
optionally, wherein the exon portion of the intron/exon junction comprises PMP22 Exon 3; and/or
optionally, wherein the exon portion of the intron/exon junction comprises PMP22 Exon 4.

8. The composition of any one of claims 1 to 7, wherein the target region of the PMP22 pre-mRNA comprises the 5′-end of an exon.

9. The composition of any one of claims 1 to 7, wherein the target region of the PMP22 pre-mRNA comprises the 3′-end of an exon.

10. The composition of any one of claims 7 to 9, wherein the target region of the PMP22 pre-mRNA spans an intron/exon junction comprising or consisting of at least 2, 3, 4, 5, 6, 7, 8, 9, 10, 11, or 12 nucleotides of the intron and a portion of the exon;
optionally, where the target region of the PMP22 pre-mRNA consists of 2, 3, 4, 5, 6, 7, 8, 9, 10, 11, or 12 nucleotides of the intron and a portion of the exon.

11. The composition of any one of claims 7 to 10, wherein the target region of the PMP22 pre-mRNA spans an intron/exon junction comprising or consisting of at least 2, 3, 4, 5, 6, 7, 8, 9, 10, 11, or 12 nucleotides of the exon and a portion of the intron;
optionally, where the target region of the PMP22 pre-mRNA consists of 2, 3, 4, 5, 6, 7, 8, 9, 10, 11, or 12 nucleotides of the exon and a portion of the intron.

12. The composition of any one of claims 1 to 11, wherein the PMP22 pre-mRNA target region comprises or consists of SEQ ID NO: 2 (Exon 3, 5′-end), SEQ ID NO: 35 (Exon 3, 3′-end), SEQ ID NO: 76 (Exon 4, 5′-end), SEQ ID NO: 111 (Exon 4, 3′-end), SEQ ID NO: 163 (Exon 2, 5′-end), and/or SEQ ID NO: 198 (Exon 2, 3′-end), or a portion or subset/fragment thereof.

13. The composition of claim 12, wherein the ASO comprises or consists of a complementary region that is complementary, or complementary except for one, two, three, four, or five mismatched nucleotides, to SEQ ID NO: 2 (Exon 3, 5′-end), SEQ ID NO: 35 (Exon 3, 3′-end), SEQ ID NO: 76 (Exon 4, 5′-end), SEQ ID NO: 111 (Exon 4, 3′-end), SEQ ID NO: 163 (Exon 2, 5′-end), and/or SEQ ID NO: 198 (Exon 2, 3′-end);
optionally, wherein the ASO comprises or consists of a nucleotide sequence of SEQ ID NOs: 3-34 (Exon 3, 5′-end), SEQ ID NOs: 37-70 (Exon 3, 3′-end), SEQ ID NOs: 77-110 (Exon 4, 5′-end), SEQ ID NOs: 112-145 (Exon 4, 3′-end), SEQ ID NOs: 164-197 (Exon 2, 5′-end), or SEQ ID NOs: 199-232 (Exon 2, 3′-end), or fragment thereof sufficient to hybridize to PMP22 pre-mRNA; or
wherein the ASO comprises or consists of a nucleotide sequence of SEQ ID NOs: 3-34 (Exon 3, 5′-end), SEQ ID NOs: 37-70 (Exon 3, 3′-end), SEQ ID NOs: 77-110 (Exon 4, 5′-end), SEQ ID NOs: 112-145 (Exon 4, 3′-end), SEQ ID NOs: 164-197 (Exon 2, 5′-end), or SEQ ID NOs: 199-232 (Exon 2, 3′-end), except for having one, two, or three nucleotide substitutions, or fragment thereof sufficient to hybridize to PMP22 pre-mRNA.

14. The composition of claim 1:
wherein the ASO comprises or consists of the nucleic acid sequence of:
SEQ ID NO: 71 (SHC-006 25-mer),
SEQ ID NO: 72 (SHC-001 24-mer),
SEQ ID NO: 73 (SHC-005 25-mer),
SEQ ID NO: 74 (SHC-010 21-mer),
SEQ ID NO: 75 (SHC-012 20-mer),
SEQ ID NO: 146 (SHC-029 21-mer),
SEQ ID NO: 147 (SHC-028 20-mer),
SEQ ID NO: 148 (SHC-027 20-mer),
SEQ ID NO: 149 (SHC-031 21-mer),
SEQ ID NO: 150 (SHC-030 20-mer),
SEQ ID NO: 151 (SHC-032 20-mer),
SEQ ID NO: 156,
SEQ ID NO: 159,
SEQ ID NO: 162,
SEQ ID NO: 235, or
SEQ ID NO: 238;
or wherein the ASO comprises or consists of the nucleic acid sequence of:
SEQ ID NO: 71 (SHC-006 25-mer),
SEQ ID NO: 72 (SHC-001 24-mer),
SEQ ID NO: 73 (SHC-005 25-mer),
SEQ ID NO: 74 (SHC-010 21-mer),
SEQ ID NO: 75 (SHC-012 20-mer),
SEQ ID NO: 146 (SHC-029 21-mer),
SEQ ID NO: 147 (SHC-028 20-mer),
SEQ ID NO: 148 (SHC-027 20-mer),
SEQ ID NO: 149 (SHC-031 21-mer),
SEQ ID NO: 150 (SHC-030 20-mer),
SEQ ID NO: 151 (SHC-032 20-mer),
SEQ ID NO: 156,
SEQ ID NO: 159,
SEQ ID NO: 162,
SEQ ID NO: 235, or
SEQ ID NO: 238,
except for having one, two, or three nucleotide substitutions.

15. A method of decreasing the amount of full-length PMP22 mRNA expression in a cell, the method comprising administering to the cell a composition comprising an antisense oligonucleotide (ASO) of any one of claims 1 to 14;
optionally, wherein an PMP22 exon-skipped mRNA is produced; and/or
optionally, wherein the amount of functional PMP22 protein produced in the cell is decreased.

16. The method of claim 15,
wherein the amount of full-length PMP22 mRNA in the cell is decreased by at least about 5%, 10%, 15%, 20%, 25%, 30%, 40%, 50%, 60%, 75%, or 95% in response to the ASO,
and/or
wherein the amount of PMP22 mRNA in the cell is decreased by not more than about 25%, 30%, 40%, 50%, 60%, 70%, 80%, 90%, or 95% in response to the ASO.

17. The method of claim 15 or 16, wherein the amount of full-length PMP22 mRNA in the cell is decreased by from any of about 5%, 10%, 15%, 20%, 25%, 30%, 40%, 50%, 60%, or 75% to any of about 25%, 30%, 40%, 50%, 60%, 70%, 80%, 90%, or 95% in response to the ASO.

18. The method of any one of claims 15 to 17,
wherein the amount of functional PMP22 protein in the cell is decreased by at least about 5%, 10%, 15%, 20%, 25%, 30%, 40%, 50%, 60%, 75%, or 95% in response to the ASO,
and/or
wherein the amount of functional PMP22 protein in the cell is decreased by not more than about 25%, 30%, 40%, 50%, 60%, 70%, 80%, 90%, or 95% in response to the ASO.

19. The method of claim 17 or 18, wherein the amount of functional PMP22 protein in the cell is decreased by from any of about 5%, 10%, 15%, 20%, 25%, 30%, 40%, 50%, 60%, or 75% to any of about 25%, 30%, 40%, 50%, 60%, 70%, 80%, 90%, or 95% in response to the ASO.

20. The method of any one of claims 15 to 19, wherein the method comprises targeting a junction between an intron and an exon within a PMP22 pre-mRNA.

21. A method of producing an exon-skipped PMP22 pre-mRNA, the method comprising administering to a cell a composition comprising an antisense oligonucleotide (ASO) of any one of claims 1 to 14.

22. The method of claim 21, wherein the amount of full-length PMP22 mRNA expression in the cell is decreased;
optionally, wherein the amount of functional PMP22 protein produced in the cell is decreased.

23. The method of claim 21 or 22, wherein the method comprises targeting a junction between an intron and an exon within a PMP22 pre-mRNA.

24. A method of treating Charcot-Marie-Tooth disease, the method comprising administering to a subject in need thereof a composition comprising an antisense oligonucleotide (ASO) of any one of claims 1 to 14.

25. The method of claim 24, wherein the ASO is administered as a pharmaceutically acceptable salt.

26. The method of claim 24 or 25, wherein the ASO is administered in a pharmaceutically acceptable carrier or diluent.

27. The method of any one of claims 24 to 26, wherein at least one symptom of the disease is alleviated.

28. The method of any one of claims 24 to 26, wherein the rate of progression of at least one symptom of the disease is decreased.

29. The method of any one of claims 24 to 28, wherein the dosing regimen for administering the composition is based on the age of the subject.

30. The method of any one of claims 24 to 29, wherein the dosing regimen for administering the composition is based on the symptom progression of the subject.

31. The method of any one of claims 24 to 30, wherein the dosing regimen for administering the composition is based on the overall body weight of the subject.

32. The method of any one of claims 24 to 31, wherein the dosing regimen for administering the composition is based on the physical performance of the subject.

33. The method of any one of claims 24 to 32, wherein a subject is given a higher dose or a loading dose of the composition based on greater symptom severity and/or older age for a period of time, which is later changed to a lower does.

34. A composition comprising an antisense oligonucleotide (ASO), wherein the ASO comprises or consists of a complementary region that is complementary, or complementary except for one, two, three, four, or five mismatched nucleotides, to a target region of a PMP22 pre-mRNA;
wherein the target region of the PMP22 pre-mRNA comprises an intron/exon junction of one of the coding exons.

35. The composition of claim 34,
wherein the ASO comprises or consists of a complementary region of at least about 12, 13, 14, 15, 16, 17, 18, 19, 20, 21, 22, 23, or 24 contiguous nucleotides that are complementary, or complementary except for one, two, three, four, or five mismatched nucleotides, to the PMP22 pre-mRNA target region; or
wherein the PMP22 pre-mRNA target region comprises two separate segments of the PMP22 pre-mRNA, and optionally, wherein the ASO comprises or consists of a complementary region of at least about 6, 8, 9, 10, 11, or 12 nucleotides that are complementary, or complementary except for one, two, or three mismatched nucleotides, to a first segment of contiguous sequence of the PMP22 pre-mRNA target region and wherein the ASO comprises or consists of a complementary region of at least about 6, 8, 9, 10, 11, or 12 nucleotides that are complementary, or complementary except for one, two, or three mismatched nucleotides, to a second segment of contiguous sequence of the PMP22 pre-mRNA target region.

36. The composition of claim 34 or 35, wherein the ASO comprises or consists of a complementary region between any of about 12, 13, 14, 15, 16, 17, 18, 19, 20, 21, 22, 23, 24, 25, 30, 35, 40, or 45 and any of about 13, 14, 15, 16, 17, 18, 19, 20, 21, 22, 23, 24, 25, 26, 27, 28, 29, 30, 35, 40, 45, or 50 contiguous nucleotides that are complementary, or complementary except for one, two, three, four, or five mismatched nucleotides, to the PMP22 pre-mRNA target region;
optionally, wherein the ASO comprises or consists of a complementary region of 16, 17, 18, 19, 20, 21, 22, 23, 24, or 25 contiguous nucleotides that are complementary, or complementary except for one, two, three, four, or five mismatched nucleotides, to the PMP22 pre-mRNA target region.

37. The composition of any one of claims 34 to 36,
wherein the exon portion of the intron/exon junction comprises PMP22 Exon 3; and/or
wherein the exon portion of the intron/exon junction comprises PMP22 Exon 4.

38. The composition of any one of claims 34 to 37, wherein the target region of the PMP22 pre-mRNA comprises the 3′-end of an exon.

39. The composition of any one of claims 34 to 37, wherein the target region of the PMP22 pre-mRNA comprises the 5′-end of an exon.

40. The composition of any one of claims 34 to 39, wherein the target region of the PMP22 pre-mRNA comprises an intron/exon junction comprising at least 2, 3, 4, 5, 6, 7, 8, 9, 10, 11, or 12 nucleotides of the intron and a portion of the exon;
optionally, where the target region of the PMP22 pre-mRNA comprises or consists of 2, 3, 4, 5, 6, 7, 8, 9, 10, 11, or 12 nucleotides of the intron and a portion of the exon.

41. The composition of any one of claims 34 to 40, wherein the target region of the PMP22 pre-mRNA comprises an intron/exon junction comprising at least 2, 3, 4, 5, 6, 7, 8, 9, 10, 11, or 12 nucleotides of the exon and a portion of the intron;
optionally, where the target region of the PMP22 pre-mRNA comprises or consists of 2, 3, 4, 5, 6, 7, 8, 9, 10, 11, or 12 nucleotides of the exon and a portion of the intron.

42. The composition of any one of claims 34 to 41, wherein ASO is a modified and/or synthetic oligonucleotide;
optionally, wherein the ASO is a phosphorodiamidate morpholino oligomer (PMO).

43. The composition of any one of claims 34 to 42, wherein the PMP22 pre-mRNA target region comprises or consists of SEQ ID NO: 2 (Exon 3, 5′-end), SEQ ID NO: 35 (Exon 3, 3′-end), SEQ ID NO: 76 (Exon 4, 5′-end), SEQ ID NO: 111 (Exon 4, 3′-end), SEQ ID NO: 163 (Exon 2, 5′-end), and/or SEQ ID NO: 198 (Exon 2, 3′-end).


SEQUENCE LISTING (239 sequences)

SEQ ID NO: 1 - DNA, 31748 bp, Homo sapiens
  source 1..31748: mol_type = genomic DNA
gttttgcctaggctaggaggagcccaatcccagaccatgctatccagtagagttacgggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggtaagtgatttggtggggagagtgaaggagatggaggagaaatgtgggcatcttccagtgagggtgccagaaagcgcagcgcaggcgcggggctttggccagctccctggggcttctgtttaggggcacagggtcccctgtgtgtcctgtttccctccaatgggtcttggagtaatacaacgaggagagcctttatggtctataagaccttacagggcaagggtgatggtgctggtgtctggatagcggataagggcggcccagttctcgccttgctgaagaggcgttgactctgggacacactggcaaaacagtccctgtggcggacatcccgagagtgttggactcctgtggttccccagttcccagcctcctgccatcggcttaattcaaaccctctgggagtcatcagaaatccttgtttatttcttccagtgcattccagagtttctttgaatgtgatgcttgtggagaggaacagaggggcctgggattgggtactttccaaactgggttattgaaacgttgtctccctggcccaagactctgcctaaaatggtccccagagtctcagccagatctttcacagccatgcatgtcctctaacaggtcatcccagccttcagttcccccaaagtttaaaatgaggggcgaggaccgcattacttgggataaatgtcctgccggctctgaccggctgaaattctcggactcagccttttaccctctgtctctctctcccgtccgctggacatctactcccttcggtccaggctcctgggctcctgtcccaccgcacccagaccggagctcaggcttgttggggttcgtgtcctggctttgagtgcctggggtgcagactggacccctaggcgagcacgtgggtctagcgcaagagcagaatgccccctgaccccaggggctgcctggggaagcgcgcggtggacgggaagcgcagggtccgggcaaccctctcgagccattcttttctttccacactactctggctctgcaccacctctccggagccgccagagtctgcgcgaggctgagctggggccaggaccgttcctctacgctggcagagttgggtggaaacttggagacaaacggaatgcggagagcactggggtctgggaaaccagcctcctcgccgctgtctcccccaccgcatgccacggctgtctccaggtctcagacggaaatctgggcacctactcccctcacccctacctccaccccataggggaattcaccatctgaaccggggtctcggagaacgtgacctctcaccttccagggaggtcgccgggaggtgcttggggtgggtggaagcgtgcagtggcctctgctcatattttctggaaacccctccgttccctgggtggttttagacgtgcgaaccgcttgttttgtttccaaaagcaaaagatgttccgttgcaggcgggcccggctgggcgctgggcactgggcgctggtcctgcaggcggctgctgccccctctcggcggcaggcggcgcgaaggctcctgacccgcgcgggcggtcgggctgcgggcgctgggccaggccgggccttccgctagtgcgcgggaccctccctctgcgcgcgcctccgtcgctcggcccagtgcgttcggcctcacgcccagcgctctcctcgcagatgctcctcctgttgctgagtatcatcgtcctccacgtcgcggtgctggtgctgctgttcgtctccacgatcgtcagcgtgagtgcctggcggggaggctccctgcgcggcccgcccttccccatctgggttcccagcccgtgctcctgctggttcaggactgtgttatttgcagacagttggaagtctcagacgtcccagggaagtttctggcaatctgcccccttccagttgctttgagaaaacgagagcagattcagtgataggaaccaagcgagcgctgggctgggttagctgcgcaggtctctatttaagccaagtaacttcagagccgatctagggtccccgaccttcatttgacaagattgtttaacttttttttttcttgatgcagcctcgttgaagaagaggagtatttatgatttttttttctccaaaccatagtcagatgggtgaattaatttataaaacacccttttaggacttgaaattggaaagttaaaatgggcttttctggaaaagagttcacaggtcccatgtcggctttatggcatgaacacaacacattgttagtaaagcttccactggtaggaacaggccatcagagtaacttctgttacaaaactgtccagcccattagatcttaaagttattttcttggcgatgaagatgagcagacattcaggcagttttccagtggtggctttcacttcttaatttaaggcacattgtaagcatgattttctttttcgttttcttttcttttcttttctttttttttttttttttgagatggagtctccagcctgtcacccgggctggagtgcagtgccatgatcttggcttactgcaacctctgccttccgggttcaagccattctcctgcctcagcctgggattacaggtgcccgccaccacgcctggctaattttttttttttttgtatttttagtagagacggggtttcactatgttggccaggcttgtctcaaactcctgacctcatgatccgcccacctctgcctcccaaagtgctgggattacaggtgtgagccaccgtgcccggcctgtaagcatgattttctagtctctgtgagaaataattatgttggggatttgtgacttagtttaacatttacaagcatcttcacagtatctccattgtgcctggtgatgatgatatcagactgataggagctataaacaaggaaatagactgagagaggcgggcttggggaatgatcccgagatgctgggaggagagaaggcttgaatgcaggtgtctaaggttgagttcatggctccttctactatagtaaacatctctgcacataatcgtttctgtgtgcatgtggaacttctccatttacaaggtgcttttaagtcataaaacgttggctcttaccatgcaggggtgggcggtgtggctaggtggatgcgggtgcttttcgccatccctgggcctttctccttccccttttccttcactcctccctccctccctgactcaggatatctatctgattctctctagcaatggatcgtgggcaatggacacgcaactgatctctggcagaactgtagcgcctcttcctcaggaaatgtccaccactgtttctcatcatcaccaaacggtgaggctggttttgtgctccatgagcttgtcctcagacgcctgtaacacgtttctcagcccagagctggaaacgcttattggaagacacaagccagaatgtcgttgctggggtggggtgggatgtgacagggagccatgagtccagggaaaatgggaggggtggtgatctcgctggggaagctgagatcctgggcatcattgtgattcactctacctagagacatgcccttagtgccctcctgatcacttaggataatgctttttgtgattgaaaaaaatatcgacctgggagctgtgaaggtggtatgatagctaagcctttggcagacccccttgggacattggatcaaacgtgtatctttggtttggtggctgtctctattcagaaatgtcctgcccctttctgctgcagtgacagtggggctagctcactgcaccgtcagttacatggacacaagactggctgcctttccaggtcttgggcctcagtgatgccacctagcatcaaaacctttgaatgtttccttgttgcaagatggggacttgaagctatttgcaaaaccctaagctaagccttggggtccttaatccagaagatcgtatttctcctctgtcctcagcatctttgcttgtgagatatggaagccaggcttcatgagggtcaaattaagggataattggcagcagaagcaccaactaggtacccaaggcacccagagggaaaggaacacccagccaggagccctgtgtgtttggatgcagacaggtggaggtaaacaggatgagctgttttggtggctgggtaggccatagcgattgatgttaagagcccgtggacctggatccacatgatcttttgttccagaagatgtgtcctcaggctggaaggacctagagatacagcagagctttcctaacatcctgggtttaaaaggccacggaaatacagctttaaatgttgctggagctactcatagatccagtatatatgttcaagatgctacaccagacgagagaaaccagtttagatccagttcagtaaaacaggcagtgaagtggcctttggtcctggctttgcataatccagttgctttgctggactttccctcttacagctcagtacccagtaatacccaagcatgtccccagctgaagcattataaaccaaattccttgaaccacagtacaaatagaaactgctaaaataataatttggtaatttacatggagaagatacgcaaggagtagaaaaccctgctttatgtttttaccttctgggttggggagggagcagagaaaaggtcccagtcctattggagtgagaacagtgcttaatcttgtctctgagtgtgtgaaaggcattgaattaaagccatcccagaaattacgtggtgggtttgcatggtgagttggcaactgagtaaatgaaatactcatgagtgcctgagaatgtgaccgtagcactgcttgtccccaaagccacagcatccggtttctccccctttagggtctggctgaagttcccggagcactccggatgggagtctccgtactcgctactgacacctggtggttacaagtagtgactcagcccgaaaagggcaggctttggtgctcacaggcgccatccccaagtggcatctggccacgcggcttggcacagaatcctggactcttgggcccggaagggggcagttttggcaggcgtgttaaaacccggcgaagtttcagcagaaacgaggcggcagaggagttgctaagttgtgcttaaaccatctgcaggaaagaaagcaatattgacctgtagcatgttacagattgacatttggtgtcttctgccgttggaaatagctgtcagttggtgctagaagcagatctcaaagaggcactagggttattttaatcaggaaacttcttatctctcaacttgttccttctgtgccaagctcatgttcttgagttcatttacaatgcctccttagtgtgggtttcaaatctgcctcgttgctttccgtgaggaccccagagtgtctgttttccacatctcccttctcagctctcacaatcagggcattttgaaaacatctggacactgcacctgaacatgctgggtttgttttcacgccactcaggcttaatctaaatttgaaatttccatttacacttccccagtagtggttatctctgccgctatcttccccaacgtggcagcacttgctacgacttcagccttaagtggcgaatcctccagggcctctttgattgagtttaacaattgtggctgcagacaataagctgaaaaaaatgtgtactttttaaaacattatactgtctttacaaatgaactatgctcattgcgaaccatttaacttgtctgtatttgctcttagaaagtatatttgaaaaaaaatacttgttaagtaatcaaaagtaatttgttattgacatcgatttgtgaagaatatgttgcaagtgtgaaaaaataaatagtggtgcaatctcggctgactacaacctccacctcctgggttcaagcgattttcctgcctcagcctcccgagtagctgggattataggcgcccaacaccacgcctggctaatttttgtatttttagtagagaggggtttcatcatgttgggcaggctggtctcgaactcctgacctcaggagatccacccacctcacctcccaaagtgctgggattagaggtgtgagccactgcgccatgtgacttggtatttttttctgaaaggactcatttttctggtgcatgaccatgacacttcacctctctctgggggttgccactcatgttgttttctacatgaatggggcctcctggcatcgtgccatgaggcagtctaacatctaggttgtgtagtttgaaagagacacaccagatcttgcccccaggctacatcttaatattagaaatagttttagatttttaactaactgactttgactccgcctgccttctcgtttttctaaatgtgtttgtatactttctggcaccccctccttatctgcctcgtgtgggcagaggtaagagaggcttagtgtggggatattcgttatgatattgtatccagtgcctctccggcccagggaatacaaggatctggaatgcatgggccttgccttgatggtcccgagcctggtcgaagagtcaggagatagatatggaagggtgaatcactcccttggaaggcagagcaggccaggtgcccaaagggaggcaggcagacagcgtctgagttcagggaggaagcggatgggaaatctctggtgggaggtagaatctgatccatcctccaaaggatgggcaggatttaagtcaatacaggcaacggggaggagaatattccctgtgacctaaggtaagggtagaacaaggcaggggatggggtagactggttggctagtgttctggttaggattaggattgattgtgtataagagaacatcttcaaatgataggggcttaagtaagatcatttatttttctctcttttcaaagaagtctggaagtaggtagcctgagagaggtatggaggttccatgaaattgtcagagatgcagacactttctagctcatttctttgccatctcttgggtttggctgtcatcttcaggaaacaaaaggctgctagagctccagccatctcatccatgattcaagcagtagggtggagaaaggaggtggggaagaatgaatctccttttcttttaatgaaatgtgacatggtcacacctacacctatttacaagagaggctgagaaatatagtcattcggccttgggggaagcatggcttttcaaatcatagaaacctggcagcaaatacctctctgtaagcctggtgtggaatactttccttgagggtttgaccatcccctcccatacccaatcatactcttttgcaatagctcagttgggccctatgcagttccataagtcccagttatttcagcattcttcaaagagtccatctctgctgcatcctgggctctgtcagccaccccaccattctcacatgggctcttctcagaaactttaggacctgttgaggcttctctctctctatttctaccacctctccaggtataagtaagtcttattattattattgagacagagtttcactcttgttgcccaggctggagttcagtggcgccatcttggctcactgcaacctccgcttcccgggttcaagcaattctcctgcctcagcctcccgggtagctgggattacaggcatgcgccaccacgcttggctaattttttgtatttttagtagagacgggctttctccatgttggtcaggctggtctcgaactcccgacctcaggtgatccacctgcctcagcctcccaaagtgctgagattacaagcgtgagccaccatgcctggccatcttattattatttaatgagtccagacctctcttctgaatcccagacctgtttatccagctgtctcctggccacttcccttgggatatctaagagaggcccccaagtcactaggccagagacagtccttatcttcctctgccaattcaacccctctgtcactttctagtattttagtgaatgctgcttcaccagccagcaagccaaacatctagagtctgccttgacttgacgcgtttccttctttctcttctcagcattgtcacgggtccctcaggccaccccattgttccaatgcctgttgcccacctgtggtcacgctgcttcctcgtttgtcagaggcgccagggcagcctcctaactagtttgcctgaaccctttcttgctcctctgtgactcatttttcatatggaatccaggctgatatttaaaaaagcacaaacctaattatatcactcctcattttgaaacctttcagaggccgttcattggtcttaatcttcagatgaaaatctttaacaaggcccatcggaatctacctgaatgatccactcctgcttctgttaagtctcacctcccatctctcttcccctcatcttggaatttcagcgttttctcacgtcctgaaatgtgaacttcccacttcgggagtctctccatttctcttctgatctttcatgcaggactatttcttgaacaactctcgtgtaccagagactattgcaggtgctaaggttacaagagtgaacaagagcttatgttttactggacaaaccagtcagtaaacatggagaatttgggggcaatggtaagaactatataagacagggtaatgggataaagagggattgcagtggtgaaggaaggtctccttgaggagtggccaggagccagccatatgaacatctcggggaggttccatacagaaggaccgtcaagtgcaagatcgctggggagggaaggagcttggcgtagttgagggacagagggcagggcagactagtgggaacagagagtgaggggtaagtagaatcacagaccatctcaaaggtaccccccacccccactaggaaggagttgggtgcacatggatgcatttctaatctttgtgatttccttagacctgctgtcttccacgggtttctcagtggtccatttgattccaagttggagtgacagtccagaagcctctataaggcagaatatatcagaaattgtacaaggtagaaatgcatcggtatattttcagtgattattttgggtgacgggattcttatcactttggctgttttctttcttttgctgccttacctgttttcctcattgagatcttttattttcacaatcagaaaataactgtgaacattttatgttagaaaattggttttgagtagtaagtaggttccctctgccttgattaagagagtggagccctggggcatggcacgcagggtccatggcgtcgctttgaatgaggccagttcctggagctttagtttgcaaactcatacaatctggaaggctctggtgagggggaggggtgtgccaagcctcctccccaagaccctatgggtgagtttaaaatctactgacccaaaggattcaggcaaaacatttgccctgctattgaatgaaccagtgctttttgttttgaagtctccaagatccaaatatcaagtccaagttctcacaagcattcccctgctttttgtttaatttgtgtggacctgacgtgtagcagaatgggtcagaatactggaattaagccacaggcggcttgggagcccaagagctagaagggcacagtcctggcttgccctggcccctggctggtcttcatcagcttcagtgatgaggcaggacccaggcagtttgtcttcagtaaaatctgtctctcagggcctgtccctgctcacaaagtcctctcttgtctcagaagaaaaggtagttgataaactttttcatgctgaggtgttaggcacaaaaaatgtgcccctttttgaatcacttttgtttttaaaaaggaggccagtgctcaacaccaaagcatgaagggaattgttgagctttcctgtaatctgtccccaaacgtccttggccacctccattgtctaagggcagtacctgcagcccctcatttcccagtgacaacaggtgggtctgctctgccacagtgtgtgggcgggaccagcaccagacgctgagaagatgaccacttactactcttcttttccatttaaacagcagcagcagcagcagcagcagcagcagcagcagcaacagcaaactcatgactatcaataggttgtgtgaggaattagatcaatgctttgggttgaaaattagagaaactaaatcttgccagtctcagcagcccccaacctgagcccttggtggtctcctatcatcgctgtcttcaaaacgatcccagacttgtttatcaatttggtgacactgtcattttggcaaggacaccagacaatttccttgtctgtggctcagcttttggccagattttagaaaacacgggagaccttctgcatttatgggttcatgttttacattttaaaatcttcgtggctccctcagccaacccataatctctacccaaggaatggctctggattttacaggctcttatgagatgctgagaccctggtggtacaagggaggtacagaggacggaatgtgttgtcaacatgccaggatttcaacccttagggtttctctgatgccaatacccaggaattagtatgaaatgtttgtttgaacctgtgtctgaactgtatgacctggaatctatagtctttgctttaaaaaaacttcaattgtatccccaagatttcaccagcaccaagaaaacatcccttgcatttcaggagcagggactggccatggtgcagcctgaggttgtggatgttgatctttggtgcccgagagaaggctgtctgggatgaagccttctgactgtggtcaggtccctgccagtgctgggggccacatgagtgtgcagtcatccacacacaagtggcccccaacactggcagggacccggagtcatctggcccatccctccttcctatcccatcgagccacagacccctctgcaatatgcctaccacatgttcatcctaccactgatggggaacttattaccttcaagagagcctcttttgtttttgaagagctaatagtgagatccattcatgtgttcttcaactctattgagcctgtcctatgtactgggcactgttctagaaatggggtgcacagagatgaacaagaaagagcaggtccctgccattgtgcagctcacaaggagttggaggaaacaggcaatacacaatacatgcatcactatgggtagtggaacatgctctgcaggaaacaaatagggtgatccaatggaaagtcatcactggaggaatgagctgctcagggaaggcctcttccttccatagtgatgtggtctgagatttagaagacagggaaggaccagttataggaagaaccatgggaagaaaattctaggaggtaaagggaagaaccaaggccctgagttagctgaagaaaggagggtggggaatgcgggagagagggatgggagcaggatcgctggtgcctcacaggctgtggtactgagaaggacagttgggacatcattgaagggttttaagcagaagagtgtcatgatttgattcatgttttgtaagctcattctggctgttgagaatgaattctggggagaggcaagagtggaaactggggaccagtgaggagtttgtggtcatagtccagactggacatgatgatgacactgagatgacaatgatgacaatgacaatgatgttaagaatacaaacagctcacgtaagtgccaggcattctttgaaaagcaatgcatttaatcctcacaagaactctaggaggtagctggtggcttagctagggaggggaagtagagaagaaaacaggtctcatgacagggtttgctgatgggttagatgtagggagttagggaagaatgattctagatgactctacgttttgggcttgagcaactggtgtgtaactgaatcttggactgagatggggagactggaggaggaacagaagatttgcgggaaggtgtagaaatcaggatttatattttgaccatcagagaggtcagcaaatcacttcaatttagaagtctttggcttaggggagaggtctgggctggaaattcaaagtcaagagtcattcctctataccgaagggtattcaatgttgtggtgctgaatacagttacttggggaggagttgagagcagaaggcagaaaaggggaacagggttcagagctaagtcttgggcatgccaggggttaacgagggtacaggaaggagaggagactggggatgaacagccagtgatgagggaggaatgccaggagatattgtcacttaagaaattacctcggctgggcgcggtggctcacgcctataatcccagcactttgggaggctgaggcgggcggattacaaggtcaggagattgagaccatcctggctaacacggtgaaaccctgtctctactaaaaatacaaaacaaaattagccgggcgtggtggccggtgcctgtagtcccagctactcgggaggctgaggcaggagaatggcgtgaacccgggaggcggagcttgcagtgagccgagatcgcaccactgcacgccagcctgagcgacagagtgagactccgtctcaaaaaaagaaattaccttgttaaacccaacactgcccctcttttgtccattcacttctagttgccttcattcatgcctgatgtgctggacttgggttttatcaagttgcttggcacgtccttaaagaatttaaatctccacagtgcctcctggacaatgactgcagggtgacccccgtctctcctctacacgatgactcttcaaacagttgacgattgcagtttttctttccttccttctttcagtggcttctctggtgctggtcccagctgcataagcagggcctcttcttgcccttctgaagtttggtcagtggtttggcttggcaggctgatgagcagagaataagggaagccgctctctctttgccacaccattctccttccaccctctctagtttttgtggattctaagtctgaagaatggtggtagaataatttaattcattagattgcaagcatatcattgacttctctgcacccccagtgtgttgtagtggaaaaactagtagtctgggcatcagggaatccttgtttttctgcaccgggtctgcctgtaacaaattaggtaacctggggcaggtaccctggtttcctaaaagcagagcaggcattgaggtaggacctgaagttgaggtaggacctgcacattgcatgggtcccggagcctctgttcatgttcaggatgtaattgtggcatcgagggatcctaaaaggaggtggcctctgagctgggccttggcaatggggtagaattgcagtaggcagagggagcagagatgggttttgtgcttgtaaacgggcattggtttactgtcactggtgggggtagcagctcttttgttctagctcttcatttccataatgcgtgttcttttttcatactttaggggaaagaaggaggagaactctattatttctatggggagaattcctcctaaacctgaagatcttaaaactaagcgaattattccctgttcattctccactgatgcagttcatgactgcaattgcaactgcctttcccgtcattttctatggccaaactcagtgtttttaagtgagctcttcttttaaaaaacaaaaacaaaaacaagtctcctgataaatccgtttgaaagacactaagttttgaaagttaagcaggctttgatcattcatggtacattgtgaattaccaaggagggagattgatatccttcatcgtacaatgcactcccctcctttttcttttgtatctggtggagtaagtcttccaagagcaattcttagagaaacaaagcctactctgtttccctgtttctagagttttccaacaagggtataaaaaagcctcagacagcttactattatccagtaacctcaggtacctcagtggctgcgtgttattccctttagaagtccaaaaactcactagcagagcaagaatatggatgtcaaagtgcagaactagaccctgacaaactagctctgtgcctgccacaaagcctcttaattaggaatgcagttcattgaataactgtatcaaagttagctggaataccttgacatgaagattccttccacttactgagcagtttgtgcccactagtggccagacacaggctttgctctagctaaaatacccccaggattacctgttgagagagccgcccacgtcaccttcatcgcctttgtgagctccatgctggcacatagttgtctccatctgttttgctttccgcatgaatcataggcaaagttagcctgaccagcaagcccatttcaaagccaccagctgggggagaagttgaagcccagggggcaagacccacgctgggctttgggcatgtttgagctggtgggcgaagcatatgggcaaaggccacattgtttaggatggaggcctttcagagactcagctatttctggaatgacattcatactgagaaataaggaaaatggcgatctgtgtgatggttgtgggttgggaggtttgggcgtgggagtcctggtcttggggtcatgtgttttgaaaacagtctagcactatgcaaatgggaggtgttaataactctttgcctctgtgatttaccctctcatggctttttctctcttggcctctccaaggtctcttctgactcttgcaactgtccttcctttcactttcaacaaaacctgttctcttgagtcagaacagttttatgaattgcccagagtgggacttgatatggggcagggttggttccatgctggtaatgaaagatgcacataaactgttacattgaaaagtgcttcatactttctgagcccttaactcactaggttgtaggccctgggctaagaatatagctgagggggctgatgtatttccttcattgctcagctgctcctacagcaggaatctgaccttgaaatgagctttgatctttgtgcaactcgagcttcccactatctgctgggggagctaggtctgggtgctgccacggcccatgtcagaaagctgtcagcagctcttataccggacactctcagaacaggaggcctgcaaagagctttagctttagttttgttgttgttgttgtttttgagacagtctcgctctgtcgcccaggctggagtgcagtggcgccatctcggctcactgcaagctccacctcccgggttcacaccattctcctgcctcagcctcccaagtagttgggactacaggtgcccgccaccacgcccagctaattttttgtatttttagtagagacgaggtttcaccgtgttagccaggatggtctcaatctcctgacctcgtgatccacccgccttggcctcccaaagtgctgggattacaggtgtgagccacggcgcccggccttttttttttttttttttttgagacgagtcttgctctgtttccaggctggagtgcagtggtgtgatctcggctcactgcagtctccgccttccgggttcaagtgattctcctgctccagcctcccaaatagctgggattacaggcacatgccaccacacccagctaatttttgtatttttagtagagacggggtttcaccatgttggccagtatgatctcgatctcctgaccttgtgatccacccgctttggcctcccaaagtgctgggattacaggcatgagccaccacgcccggccagagctttagtttttaattccagtgctcagcaccaaggttggatattggcttaaaacttccactggaaactccacaaatgtttagcaggcctccactttgcaccaggaactgagtgtggcctggaggacagcaggtactcaacataagttctactatttattgcatacttgcaaattgggtggggcgtggggaagagtatcgccacactggggcttgtgttaatctatagtttagccacttgggatgttggtgtcacactaggtttgatctaaatagaacaggtttgtaattgacagcattttcacttgcattatatttctctgaactcgaatcattattttctgtacagctgacttgttttgctgtagcagagtttggcagataatagcttgcatgtcgaatccaacctgcttttgtaaataagattttattggataaatggctattgaaggaatgggtcacacccactcatttacatcttgtccatggctatggctgattttgtgatacaaaggcagaattgagtagttgttaagaagaccgtatgacctgcaaagcctaaagtatttgctctctgattctttatagatagtttgctatctgtgcagatatttgcactctgactctgtagcacccttatgctggagaggagatatgaattgataccaaagttgtatcaaaacacttttggagttctggccaatgtgctaataacctggcttgtgtgaacattgtggctctcattttcttttttcttttttttttttttgagacgagtcttgctctgtcgcccaggctggagtgcagtggcgcagtggcgcgatctcggctcactgcaagctccgcctccagggttcacgccattctcctgcctcagcctccagagtagctgggactacaggcgcccgccaccacgcccggctaatttttttttttttgtatttttagtagagacgggttttcactgtgttagccagcatggtctcgatctcctgacctcgtgatctgccctcctgggcctcccaagtgctgggattacaggcgtgacccactgcgcccggcctgtggctcccattttcatactgcttggtgtgacccagatggaaatgctccagaacctggcccctcaataacagcctgtaaatgcttgtttaccctgttttctcacatcagccttttaggtggggttatttgcactatcctttaacagcgcttcaataacaactcttttgtttgatcattatgacaacccaataaaatatattagtgcctcaagtacgtaaagcagaattcaaggccaaaaaaatgtatgtggcaagttaggtgggcggtcagaagaacttggcaacagagcccagccctgcctgggctgtcctaaccagagcacataaatgctggagagagtcaggctttgcacgtgctcacacgggacccttaccttggacaggatcaaagccctgtcatttctggagtgtgtgatatctgctgtctcttactgcgctggttgttacctgtctattccagttctgatgaatggctctgattggggagcagcatcatctgagcttagaaatgtcctcagggccctgtggggttccctttgagcaacaacgagagctcttgaatcttgcaccagccttttggagctggaatggagtttgctttctcctgggctgggctctgttttttttcctctcgctgtccttgtgtagcttttgatcaatggcactggaggaaaggaaaatcccttgttgttttcctgggcatgtggaagcccagtcctgcccagaattccactagccctttgagttttggaatcctgactcttagtaagtcaatagagtgctgcatctgtttttacctttgtaatcttttaaattggttttaattatttaagtaatacaagtattataatacatgaaaatatcctcaactgaaaggcttcttgaccaaactcttcttcgttgcaacagtagttcctgggggaacccactgacatcaatttgacacaactttggctctttctctacgaatatttgcatgcctgtggatgtctatacgcaattacatagttttttggtgtttgttttcataatgatgacatgttgtgtgtgcaccttactcttttcattgtcttggagacctttcaaagactcttcattttctaactcatgatttttttaactgctgcaaaatatttcattgtgaggatataccataatttattttccctcatagaaatgctacaatcaatgtccttatttattttcctctgagtatatgttcactgtttttccaggatatagtagataagcacttgaacacatatagtgtcattacgctagaggtgttacaatttttaaactacttttaatggcaaaaattacaattacttttgtatcaacctaatatattctgccacaaaatggctggtcacaaaaggattataataataatattattattattcctgtgaatggtgtctgcaggtagctatcacttatttgttctgctgatattatccattttttttgaattttgcctattggctggatgaaaattggtgtcaccctcctcatgggtttctcttgcatttccttggttgctagtgaggttgggcaccttttacgatgtttattggctgttcatatttctcctttgctaatcacatggcttttcaattttatacttagctgttttctctttgttctgtctgtaatttaattttgttaatgatgcgaactagagatttaattttatgttttaccaaatgaatagccaacactatgtattgtctgttgtatttttccccactggtttatatgatgttgtgtattatctaccaaatttacatgtatgcttccaaagtcttattccatcccattcttttatttttctttcatgattaacaccatatttttgaattgctaaaactttataatataaatataagctttgtatgtctataaataaaatacattttattaattataattatatgttatataattataatataagcacttgaacatatatacttctataattatatattatataattatatatcatacttctatgatatgttctataattatatattgtatatatcatagttctatgatatgttctataattatatattataaagttacaattaattgtatatttaatttataccatatactgtaataagcaaaatatagaaatatgttttaaatctggtaggtggtatcttgactcattggttttatatgtttttctgattttctttttgttcatttgttcgttttggtttttagttgctgttgtgtttacttcaaattttttcttatctgtcattgctcattgttttttagatgaatttttaattgatttttttccagttccagttgcaatatggattaggacagcattgaatttgtagattattttggatgaaattggcatctttataactttgagtgtttctatccaggaacttagtatgtttctttagtacttcttttatgtacttgaataaaatttgtctaacttgttgatgtatccccaaagaaccagacatttttattaaatgtattttaatatttttttctgtctcattagtctgtgcttttatccatattaagtcaattaatttttggacttatttcattgttttttttctggcttcttgagttgaaagtgtagttcactctttttaaggatttttctagcaaatgcttagaagtgtatacattgtcctctgagaactgctttggttacaacccacaggtgtgccatgtggctctctatataattccttgctgaagaagtttttacttttcatcttgttttttttaatccgaggaattatctggaacactgtttttaaatttccaaatttattttggctagtttgtttttaatgtctaatttctgtttgcctttaggtatatgttgagcctacatcatttctgcttctatatttattgagatattctttgttgcccttatagtcagtttgaaaagctgtcccatgtgtgtttgaaaataactttgctatatagacacaagttttctatacatctattaggacagatttgttaatcctcttatgtaatctatattctttcttctttttggtctacctgacatctattttgaggaaggaagctaaagtcttctactgttattgtgattttaacaatttcttctcatattttgattactttttgattttaactttttcaggccatgttattatatgcataatgatttattttcttcagtcatagatcatgtaaaccagaagtaaagatcaaataaaccaaatctttcctctatgaaacaaccaaacaactctctggccacttttatcctttcaacataagttctattttttattattaatttatcttctttcttttttgttaacatttgcctgaagcttcttttcagaatcttttttgtttttgactgttgcttgttttaggtgtatctattatattagaatatagctacaatgtctctcttttttttttttttttttttttttgagacagagtctctctctgtgcccaggctggagtgcagtggtgtgatcttggctcactacaacctccacctcctgagttcaagcgattctctgcctcaacctcccaagtagctgggactataggtgtccaccagcacacctggccaatttttttgtatttttcgtagagatggggttttggcatgttggccaggctggtcttgaactcctggcctcaagtgatctacctgcctcagcctcccaaaatgtcttaatcaaagataaaattccatatcttatgagtgccttgaacccattcacatataatgtggttgatgatagacttactatcttaatgcatgtgttttatttattgtgcttatggttgaaaaccatcttcttcatctctggatcatcagagtcaaccagagggcttggctcaaagcagtaggtattcaacaaatgctcattgaataaaaatgtccttgatcctctaattctgatttgattcgagaaataacttatggcaaagagctcaattgtaatggggttgggactaaaattcaagttaatgttgaaagatgtttcaagaagtatttaggttatattattaaatattagacactttctgtttatatagatttaacatataaagattttaagttttctgtgtatgtgaggtctcgtagtagtttctgctgaaaatgacaggggcgcggggttaagggtgaagacagtatctcaggattggagaaggaaatctaagatattgtctccaccagccatactcctcatgaacatgcacatggaaggacatttcccagccaaagtgaatccctttatttgttttagtttcagatttgagtctcagatttctttgtaattccagctgcatctaccaaagtgctagataatttttttattttaagcaaacaatattatgagaacattatctttattgcaaagtgctcctccagaagaacctctatgctggataaagacaaacatttgattaaggttatgtaagagtgaaatcaggatggctcacaacccattagccgtcattttcttactttaattgacaaagaaattgaaaaaatgaaagaagcatatgttgtgcttgaatgtgaagagtgctagactagaatcaaaggcctcttagatttggctgtgtcatttgggctcatgaaaacacctctggaaatctctttcagtgcaaaggagctggactagttgatctctcaggtttcttccaactctaaaatatctccagtctgtgccttggaaacatcttaggtgaaaatctaggaacagttaacctaatttgcacccttaaaattctgccatgagctgcttacaactcaaaacaagtttatcttactcagttactaattataaaccatccagatttcagagctgtgagtactgggtgaagcattgaaggtatgcttttgaagccattacatatggcagttactgagctgaaaggattaaatgctgcagcttccccagttgcccttcctccatgagagcagtgcctgcccccagcattctgtggcacttggaagacaagacagaggccaaatgcagatttttaccctgggcttccctctacagtgtggaactcaggttgtttcttctttcctccctgaaatgacatgagtttgcagcggatggtgaactgaagaaaccataggaggctctgtcttcttgcctgaatttcagttggaagcttggagatttggggttcaacagagataaggaagtgtaagccttcatcccgtctggtggttggcgatcacacaccgctctgtgctgaggctaatggccatgatcagagttgaccaaaaaaaaaaaaaaaaaaaaatacgggttgtccaagcaaattgatttccatacctatagagagcatacctttcttcatcagtattttcttccattcttccaaaaaattactttgggctctaacagccattcccgtgatctttacctctccttggggaatgcagataatttgaatagtggttttaagctatttttcttggaatacagaagttctgataagcccttcaaagacccctgagggcaagaagagggaaggtagtaggcagggctcaggccctctttaacacagacacatgtacataagtaacacatttgcaccaacactggaaggaaattcaatatattttaaaatgactttaactcctagtgatggaactatagatatttttttacagcccttccctgaatgttcacatgtgttttttatatatatgtgtgtgtcacgtatttgtatatgtgtatatacacatatatatagaaacacgtacatatttgtatgcatatttgtacatataggtatatatgtatgtaatgtgtacatattggcacatgtgtgtgtttgtatatgtatatatcagtacatatagatgtatgtatatatgtatatattttttcctccaaaataaacaataaagaatgtcttcttagattgcaaaagtgaattgtctgagttctgctaaggaaaaaggacttctgcttctgctgcctgtgaggactgtgctgtcagcagtcatggcccttcaggccctgcacctcgaggcagagccctcccccggccatggccagctctcctaaccaagtgtctctttcccccagaatggctgcagtctgtccaggccaccatgatcctgtcgatcatcttcagcattctgtctctgttcctgttcttctgccaactcttcaccctcaccaaggggggcaggttttacatactggaatcttccaaattcttgctggtaagttgtggatggtaaagtccatgtggaagcggggtgcatccaagtctgcggaatgattagtttagtagaaggatgtggcctcagaatgactgatgttcatgagtctccccactggatgctttccataaagtgagggtgggtgcttgtatgtgtgggtgtgtacctgtatgtgtcttagaacttgggacttagaactctccccttctccctggaatgagatgcatatgaaagagaacttagaggatctggaaggaaggtccccacccaagccaggcgtatcaacaggaatgaaactgcaatctggacacataatcagaggtgaatactgaggctatctgtagagcaaaggtcaggcttgagagctgtttctgtagattacattatgcctccagaaaatggccctgatgtgctaagaactagcaaagtagttatcaggtatgtgtcttccaccaataggtagtgatgaagccacactgacaaattctcaccttccttgcttccagttcctagattctactgggctttgattgactgttgtcatcctctggtgtcttcattttgacactcttgtgtcacatattgtcatttccaaacatggggctatgacaacacatgaaaacacatgagaggtctccttaatctcctgcctaaactgtcttcaagttccctctttaaatacgttattaatatgcatagtgtgcagagtcctagaaacttctgttacacagggtgacatcttccaactttgtctctggattctgcctagcatcttacatgcttacatcttacatcttacatcttacatcttgcattctgcctagcatcttacattagctcttacatgtctgtctgttgacttactgttgactgaaccagcagggcattggagagaagtaagagctagatgtagtggtggattctgtggtccaaattcatagatcacaaacttcatatgtaccagagtatgtctaggtactgggagatgttctcaattctgaccctctgagagggcaaaggatgtagcatctcttctctgagttggttgtcagaatgcccatggtaccatttcaccactctgtccccaggagcagtcattggaaggttgacgtaaatagggttgtatgggaagacacagcccaaggttagatgttggtgaccttgtctagaagacagagagttcccctttcctgaaaaaaggaagtaaatgattaaccacttctcattaaacactcaaatacaacatttcaatactcatggttttgagatttcaaaaccagacagtgctttgctacttacacatgtcttatgacaccaagccaagctcctggatggttgctggctctgttaaatgactaattatgcaaggagatatcatttctaggtacgttaaagtgaagagttacccttactcaattttcagttggaataaaaacaactgtaacatattctggggtttctttttttttttctcactcgttttagtttgatatcaaatcaaataatgatcatatccattgcatcagtggatatgccctcaagataatatggatttagaaccagaactttcataatgtatttctattgaaatgttagtttcataagcgatgattgggttttcatgcccatgtgtgagatgtgcctcgctcaaaccttgttatgatttggcacgttacccatctgatgtgaaaaaaattacattttatttgtacaggctcgttattttactgatgaataatttgagcccaccagaggataaatgaatgaccaaggtcacccagctcatgacagggacggttgagtgttacactgaatatagtgaggtacttcttatattttaaagacagaatgcaccaaaaaatttaaagaacacaaaatccaaggcagaagctctgccttttatattatcttttattggaactgatttacaatggaaggtaaatgcaaatttgcaccatgtattattctgaagttccaaacatctgtgatgaatacaagcctgtactataagacccagtcacattgaaaatatggagctgagaagaggtaagctgctgttgaatgggctccttgggatagccagtaccttcatcttcattcatcctgctgagctgtttcggctttaagttctttaacaatgtctttttagcaacatcattacatcattttaggccaaaactcaaagtccagagataagaacccttaagtcactcatgtaactgcactgtgtgttaaaagtatttcagttcagccaaacactcttctcctaggtattgcgatttaagtatatttactaatccactcttgcttcactattttcattctcctccaaagtcaatacaagatgtttagaactgtgctggaagtgcagaattccgaatgtaaaagcgcatgactttgtcctctttatcccctttacatctagctgcttacgtctcatgaaactgaattttcagttatctgttggtccacatttgaataagaaattatcttgaatttgaaatgctgagctgtaatagcagttgtaatttgtaagtcctgagagtgtcctgtcctccgttgttaatcccagtcaagatcatctgagagttggtctccagggaacctcagatctctaggatgttgcactggaaatggctgcaggatctttccactaattctgagaactgaaagagttaggaactctatttggagagttcctggttcccttatcgtgtgacagttctaagtcaattttgtcatgtggttttctgactcacagactagtaagaagttagtaattagagagctaagtagattagggttgttggagatgagaaacccacgttttgggaaaacctggcaagtgacaacttaacatcaaggaagtagtcagaaaagctaaaactgacaaacagaaggtagaagaaagtagctccacactcatgggatgtgaaatctacaagcgtgcatgcccggcaaatgcctctccaatgcactggagcgttttaagtggaaaccactagaatctctgtgtagtctccggaagtggctgtgagggctgcattatctctgcacagcttcctcttggtgggcccagctgtgatctttatggatggcacacatcagctttcaggaaaagcacatgaaaggtgctagggctcttggagctgactgtaggtttgggagttgtctgtctccttgctctagattacagctctgtgtgttgtgtggggtctccatggtttgccaaattatctcttcctcacttagccacaaggctgacagttaggaactatctcttcttgattgcattaggttggctgcttcctgaatgcatatcaaaaggctccttcctttagttcagtgctttcaacctgagctgtgcatcagaatcacctgaggtcctttaaaaaaaaaaaaaagacagtgggcggggcgcagtggctcacgcctgtaatcccagcactttgggaggctgaggtgggcagatcacttgaggtcaggagttcaagaccagcccggccaacatggtgaaaccctgtctctactaaaaataaaaataaaaaaatagccaggcgtggtggtgcgtgcctgtagtcccagctacttgggaaactgaggcagagaagaatcacttgaacctaggaggtggaggttgcagtgagccaagatcatgccactgcactccagcctgggggtgacagagtaagactgtctcaaaaaaaaagaaaaaagaaaagaaaaaagacagtattcaggtctcatccctggagactctcatttaatagttgtgggatggatccactgcctaggtgactctgatgtgcatttagggttgggaaccactgacatagccattaaactgtccctaatcccactgtaaggttttctaggatattttcccagaaataactaaaccaccttcttagagaaggaacatcctgatcctgcgtctggactttggagtcattcttattttcagaacctatagccatctattccttgacaagatctgttgggttgggttgcactagactggaaaacatcaagaaattaatctagacacagacctaaaaggaagatttgcacgttttgatttattttactgcttacacccagcatcaagctccatgtggggcacatagaggagcctgaataaaactattattggttggatgattagataattgtgtttcctcggcagaatcaaactcaagtcaaatgtgtggttaccgggattaagaacagaaagaatagaggggactcctgagtgagtttcactttctctctctttttgttattgttgttcatttgttttgttaatagaaagatcaatatagctatagagcatttaaaataaaaggatttgggccaggtgtggtggcgaatgcctgtaatcccaacactttgaaagactgaggtgagaggattgcttaaagccaggagttcaagaccagcctgggcaacaaagcgagatgccatctctacaaaaaatgatttaaaaattagtggggtgccttggagcacgcctgtggtcccagctattcaggaggctgaggcaggaggattgcttgaggctgagaggtcaagactacagtgaactatgatcaggcccctgcacttgctccagcctggtgacagagtgagaccctgcctctctaggaaaaaaaaaaaaagaatttggtggggggggattaataaaagttcagtgccatcctgttcagagtgattcaaaggtggcttaagtcaaataccacatttagaaatttttgttagggacggatgattatggatgtctcactggccatgcccaaatcagagtcaatgctatgggtggctttctaggcagcaatggtcatttggatactgaaaatgtaaaaggagtgggcttcaatcacagaaacacagagaagtctcttgctttcagaggaacaggcctcacagccccttcctgccctcccttgttgccctgaggatgaagtgggagggagaaaaaggcacccactcatcaacccaacatcatagctgcaccttccagtgaccaccctggtggtctcactgacatccctttccagccaacttctctctgacttctgccccaggctctcaaatgaaagtgcttttgcgaggttaccaaagtacactttgaagtctttaatttgttggatcactctgctcctttcaatactgttagctacttactctttgaagctctattcccctggcttctgagacatcgctgtgttcccacttgcctgagtgaacacaccaggcggtcatgctggcctctcctctctccctcaccccacagccagccttctactgaatcctgcatttgggtagcatcctccgtaggcattacgcccatttctttgtcttgactcactaaccccagtgcagctatgcaaaggcctcctcatgcaccttccagctacctgccttgtccctgcccctgaatccattttctgcaccatggccagagtgatcattctaaaacgtgcgtctgatcatgtttctcctctgctgaagtttcctcagaggcttctcatcacagcttccttaacatgtcatgtaaggccctctctttcctgacccttagtgagctaatcagcctattatatgagagtccgtgtggcttctccagttctccagtttggactcagcgcccgtctatgctcttggggcatctggtgcatttccttccctctctcgaaattccatcgaactatattgaaattatatgcaaatgtctgtcttctagacacacagtccttaaggtaggagccatgtctcacttatcttggtacatctagagcagcagctagaacagggcccgcatgcaataggcactcattaaatgttcactgaaccaaagggaatggagatgataaggatgtactgggaattccctcagctacttcctttgaccttggccccttgggtgcttccttccagaggctctgggctctaatcactctcatggggttgcagtcactgttaaaggagcttaagactttctcttcaaaagggctttaggcagaacttgcaggaagttgttgcagacctacccatcctgagacagggagaaactcttgtaagttgaatgctcagcacatttgtattgtctggacagggtcagtgtccttctgcttaaagatgacctatgctcccagactcaggcccctaccagggagtcccggtcattgccaaagagcagaacatctgcctgtgcctcagggcctcacttacccttgccacagggacctggtgatacatctccacctactggttgctgagaaagtgagtcacagtccacttactaggggtatttggcttttggagatgactactggatgacattaactgtcttgggttgcagacagagggaacccagccaaagaatcatccttttctctatttcagggtacatctattgctttacatgcagacaatcttgtaataatatattccctaaaacatcagtctcatgacaacaatcatataaagtgtgtatcctctctttggctttgtagatatctaagtttactgcttatcacggttaagcttagagtcattcacttctgagattctactaagatgaaagtcatacacattaagctgtgtagttagtcgctgcacttctctatactgcctctttctatccctcttgttcaaggatggaatctggtatctcctcttagtcagtgaaatcggggagtagctggtccactcagctcctgaggcatttctcatgccagatctggttagacagcccatttgggagccctgcctgcatgatgataagtgttgtcctgccctgctagcactctgttaacacctttctctctgccaaggtcatgtttccttttttatagccctgccttcattagataagtcagacagagcccaagagacaacttcagtatttcttaaagacaagagttagcactttggagatgcaagccgtggaagagtggggcaagataattaaggatcctggacctcaaggcaaataaagtgtgtccccagcagatcaaaactgctgttttctgcaatcaacaattaacccgcaatgatttcgctcctgacatggccatctttgctgtcttctgaacttcctgtaggtggcaggtcctggatgctgtcccattttggttccaagactattgtgtggctctcttgacaggggccagtcaagtggcagttttctcctctgagttgcaggaggacaggcttaacagggtggttcatgtggcgtattcccacttctgcactgtcttcaaacaaagttgcagtgtccagtaaaacagcagcccaaatagcagtgaggttagatgtaggtggccgggcggtgggggggcgcgggtgcattgggaactctggaactgcatgaatcccacacggcatactggatttataatagagtcactcagagcttctcaaagaccctgttttcagggaggaaacagaccaggctttgtgcagctccaggctggggtgctgacatcactggacccctccttgtggggggacgctgtctgtcacgtgagctggggccatcagtttgagcgtggctgctccacagggcccaagatggaggcttttcttctctttccaacaccgaagggctgcattggtgggccagggagggtggcctgacccacctgcatatccagtgccctttagccttctttgggcttcaaatgcaaccttggggcacccagggaaatggagcttgcctttgatcatcttccccctctgcagcgtggcttcccagtcatactgtcggctgcatccttattcctcactttctttttccctctccacacatttctttgtctgtttggcagtgcccataggcattgcttgcttcacctggtatgtggcaactctgttagggggaccataggcccctgtggtctgcatggagcttaatccttttaccatggcgtgactccaataatggtaaaatgtcacttcacttccattggctttaggtaatgccaccactcacttgtagagtggcatatagatttccaggggtttccatacccaaccacccttggaaagagacaggtaggtttttcttgcagcctgaatgcctgctggccatgtagtgtggcctgggaattggtgacagtgaaagaaagagaaatagggcagaaaatgtaaagaaaaccctcaggtattaaggcctaggtggccaagattggaaatagagagcaagttgttttccttgttcccatatttttgtgctacctcctttcaattctggacctggaagcagatttccttccagaggtagaaatgttcttcctacccagcaattgtcagcatccggggtggcggagagggggccgctctgccatggactctccgtcacccagcttgtccctctccttccccaggtctgtgcgtgatgagtgctgcggccatctacacggtgaggcacccggagtggcatctcaactcggattatcctacggtttcgcctacatcctggcctgggtggccttccccctggcccttctcagcggtgtcatctatgtgatcttgcggaaacgcgaatgatctcttgagtttttgtgggaacttgggtgtgggtacaaggtgcctgttgg

SEQ ID NO: 2 - DNA, 58 bp, Homo sapiens
  source 1..58: mol_type = genomic DNA
tcaggatatctatctgattctctctagcaatggatcgtgggcaatggacacgcaactg

SEQ ID NO: 3 - DNA, 25 bp, Homo sapiens
  source 1..25: mol_type = other DNA
agtcctatagatagactaagagaga

SEQ ID NO: 4 - DNA, 25 bp, Homo sapiens
  source 1..25: mol_type = other DNA
gtcctatagatagactaagagagat

SEQ ID NO: 5 - DNA, 25 bp, Homo sapiens
  source 1..25: mol_type = other DNA
tcctatagatagactaagagagatc

SEQ ID NO: 6 - DNA, 25 bp, Homo sapiens
  source 1..25: mol_type = other DNA
cctatagatagactaagagagatcg

SEQ ID NO: 7 - DNA, 25 bp, Homo sapiens
  source 1..25: mol_type = other DNA
ctatagatagactaagagagatcgt

SEQ ID NO: 8 - DNA, 25 bp, Homo sapiens
  source 1..25: mol_type = other DNA
tatagatagactaagagagatcgtt

SEQ ID NO: 9 - DNA, 25 bp, Homo sapiens
  source 1..25: mol_type = other DNA
atagatagactaagagagatcgtta

SEQ ID NO: 10 - DNA, 26 bp, Homo sapiens
  source 1..26: mol_type = other DNA
tagatagactaagagagatcgttacc

SEQ ID NO: 11 - DNA, 26 bp, Homo sapiens
  source 1..26: mol_type = other DNA
agatagactaagagagatcgttacct

SEQ ID NO: 12 - DNA, 26 bp, Homo sapiens
  source 1..26: mol_type = other DNA
gatagactaagagagatcgttaccta

SEQ ID NO: 13 - DNA, 26 bp, Homo sapiens
  source 1..26: mol_type = other DNA
atagactaagagagatcgttacctag

SEQ ID NO: 14 - DNA, 27 bp, Homo sapiens
  source 1..27: mol_type = other DNA
tagactaagagagatcgttacctagca

SEQ ID NO: 15 - DNA, 27 bp, Homo sapiens
  source 1..27: mol_type = other DNA
agactaagagagatcgttacctagcac

SEQ ID NO: 16 - DNA, 27 bp, Homo sapiens
  source 1..27: mol_type = other DNA
gactaagagagatcgttacctagcacc

SEQ ID NO: 17 - DNA, 27 bp, Homo sapiens
  source 1..27: mol_type = other DNA
actaagagagatcgttacctagcaccc

SEQ ID NO: 18 - DNA, 27 bp, Homo sapiens
  source 1..27: mol_type = other DNA
ctaagagagatcgttacctagcacccg

SEQ ID NO: 19 - DNA, 27 bp, Homo sapiens
  source 1..27: mol_type = other DNA
taagagagatcgttacctagcacccgt

SEQ ID NO: 20 - DNA, 27 bp, Homo sapiens
  source 1..27: mol_type = other DNA
aagagagatcgttacctagcacccgtt

SEQ ID NO: 21 - DNA, 27 bp, Homo sapiens
  source 1..27: mol_type = other DNA
agagagatcgttacctagcacccgtta

SEQ ID NO: 22 - DNA, 27 bp, Homo sapiens
  source 1..27: mol_type = other DNA
gagagatcgttacctagcacccgttac

SEQ ID NO: 23 - DNA, 27 bp, Homo sapiens
  source 1..27: mol_type = other DNA
agagatcgttacctagcacccgttacc

SEQ ID NO: 24 - DNA, 27 bp, Homo sapiens
  source 1..27: mol_type = other DNA
gagatcgttacctagcacccgttacct

SEQ ID NO: 25 - DNA, 27 bp, Homo sapiens
  source 1..27: mol_type = other DNA
agatcgttacctagcacccgttacctg

SEQ ID NO: 26 - DNA, 27 bp, Homo sapiens
  source 1..27: mol_type = other DNA
gatcgttacctagcacccgttacctgt

SEQ ID NO: 27 - DNA, 27 bp, Homo sapiens
  source 1..27: mol_type = other DNA
atcgttacctagcacccgttacctgtg

SEQ ID NO: 28 - DNA, 27 bp, Homo sapiens
  source 1..27: mol_type = other DNA
tcgttacctagcacccgttacctgtgc

SEQ ID NO: 29 - DNA, 27 bp, Homo sapiens
  source 1..27: mol_type = other DNA
cgttacctagcacccgttacctgtgcg

SEQ ID NO: 30 - DNA, 27 bp, Homo sapiens
  source 1..27: mol_type = other DNA
gttacctagcacccgttacctgtgcgt

SEQ ID NO: 31 - DNA, 27 bp, Homo sapiens
  source 1..27: mol_type = other DNA
ttacctagcacccgttacctgtgcgtt

SEQ ID NO: 32 - DNA, 27 bp, Homo sapiens
  source 1..27: mol_type = other DNA
tacctagcacccgttacctgtgcgttg

SEQ ID NO: 33 - DNA, 27 bp, Homo sapiens
  source 1..27: mol_type = other DNA
acctagcacccgttacctgtgcgttga

SEQ ID NO: 34 - DNA, 27 bp, Homo sapiens
  source 1..27: mol_type = other DNA
cctagcacccgttacctgtgcgttgac

SEQ ID NO: 35 - DNA, 58 bp, Homo sapiens
  source 1..58: mol_type = genomic DNA
gtccaccactgtttctcatcatcaccaaacggtgaggctggttttgtgctccatgagc

SEQ ID NO: 36 - DNA, 58 bp, Homo sapiens
  source 1..58: mol_type = other DNA
caggtggtgacaaagagtagtagtggtttgccactccgaccaaaacacgaggtactcg

SEQ ID NO: 37 - DNA, 25 bp, Homo sapiens
  source 1..25: mol_type = other DNA
caggtggtgacaaagagtagtagtg

SEQ ID NO: 38 - DNA, 25 bp, Homo sapiens
  source 1..25: mol_type = other DNA
aggtggtgacaaagagtagtagtgg

SEQ ID NO: 39 - DNA, 25 bp, Homo sapiens
  source 1..25: mol_type = other DNA
ggtggtgacaaagagtagtagtggt

SEQ ID NO: 40 - DNA, 25 bp, Homo sapiens
  source 1..25: mol_type = other DNA
gtggtgacaaagagtagtagtggtt

SEQ ID NO: 41 - DNA, 25 bp, Homo sapiens
  source 1..25: mol_type = other DNA
tggtgacaaagagtagtagtggttt

SEQ ID NO: 42 - DNA, 25 bp, Homo sapiens
  source 1..25: mol_type = other DNA
ggtgacaaagagtagtagtggtttg

SEQ ID NO: 43 - DNA, 25 bp, Homo sapiens
  source 1..25: mol_type = other DNA
gtgacaaagagtagtagtggtttgc

SEQ ID NO: 44 - DNA, 25 bp, Homo sapiens
  source 1..25: mol_type = other DNA
tgacaaagagtagtagtggtttgcc

SEQ ID NO: 45 - DNA, 25 bp, Homo sapiens
  source 1..25: mol_type = other DNA
gacaaagagtagtagtggtttgcca

SEQ ID NO: 46 - DNA, 25 bp, Homo sapiens
  source 1..25: mol_type = other DNA
acaaagagtagtagtggtttgccac

SEQ ID NO: 47 - DNA, 25 bp, Homo sapiens
  source 1..25: mol_type = other DNA
caaagagtagtagtggtttgccact

SEQ ID NO: 48 - DNA, 25 bp, Homo sapiens
  source 1..25: mol_type = other DNA
aaagagtagtagtggtttgccactc

SEQ ID NO: 49 - DNA, 25 bp, Homo sapiens
  source 1..25: mol_type = other DNA
aagagtagtagtggtttgccactcc

SEQ ID NO: 50 - DNA, 25 bp, Homo sapiens
  source 1..25: mol_type = other DNA
agagtagtagtggtttgccactccg

SEQ ID NO: 51 - DNA, 25 bp, Homo sapiens
  source 1..25: mol_type = other DNA
gagtagtagtggtttgccactccga

SEQ ID NO: 52 - DNA, 25 bp, Homo sapiens
  source 1..25: mol_type = other DNA
agtagtagtggtttgccactccgac

SEQ ID NO: 53 - DNA, 25 bp, Homo sapiens
  source 1..25: mol_type = other DNA
gtagtagtggtttgccactccgacc

SEQ ID NO: 54 - DNA, 25 bp, Homo sapiens
  source 1..25: mol_type = other DNA
tagtagtggtttgccactccgacca

SEQ ID NO: 55 - DNA, 25 bp, Homo sapiens
  source 1..25: mol_type = other DNA
agtagtggtttgccactccgaccaa

SEQ ID NO: 56 - DNA, 25 bp, Homo sapiens
  source 1..25: mol_type = other DNA
gtagtggtttgccactccgaccaaa

SEQ ID NO: 57 - DNA, 25 bp, Homo sapiens
  source 1..25: mol_type = other DNA
tagtggtttgccactccgaccaaaa

SEQ ID NO: 58 - DNA, 25 bp, Homo sapiens
  source 1..25: mol_type = other DNA
agtggtttgccactccgaccaaaac

SEQ ID NO: 59 - DNA, 25 bp, Homo sapiens
  source 1..25: mol_type = other DNA
gtggtttgccactccgaccaaaaca

SEQ ID NO: 60 - DNA, 25 bp, Homo sapiens
  source 1..25: mol_type = other DNA
tggtttgccactccgaccaaaacac

SEQ ID NO: 61 - DNA, 25 bp, Homo sapiens
  source 1..25: mol_type = other DNA
gctttgccactccgaccaaaacacg

SEQ ID NO: 62 - DNA, 25 bp, Homo sapiens
  source 1..25: mol_type = other DNA
gtttgccactccgaccaaaacacga

SEQ ID NO: 63 - DNA, 25 bp, Homo sapiens
  source 1..25: mol_type = other DNA
tttgccactccgaccaaaacacgag

SEQ ID NO: 64 - DNA, 25 bp, Homo sapiens
  source 1..25: mol_type = other DNA
ttgccactccgaccaaaacacgagg

SEQ ID NO: 65 - DNA, 25 bp, Homo sapiens
  source 1..25: mol_type = other DNA
tgccactccgaccaaaacacgaggt

SEQ ID NO: 66 - DNA, 25 bp, Homo sapiens
  source 1..25: mol_type = other DNA
gccactccgaccaaaacacgaggta

SEQ ID NO: 67 - DNA, 25 bp, Homo sapiens
  source 1..25: mol_type = other DNA
ccactccgaccaaaacacgaggtac

SEQ ID NO: 68 - DNA, 25 bp, Homo sapiens
  source 1..25: mol_type = other DNA
cactccgaccaaaacacgaggtact

SEQ ID NO: 69 - DNA, 25 bp, Homo sapiens
  source 1..25: mol_type = other DNA
actccgaccaaaacacgaggtactc

SEQ ID NO: 70 - DNA, 25 bp, Homo sapiens
  source 1..25: mol_type = other DNA
ctccgaccaaaacacgaggtactcg

SEQ ID NO: 71 - DNA, 25 bp, Homo sapiens
  source 1..25: mol_type = other DNA
atagatagactaagagagatcgtta

SEQ ID NO: 72 - DNA, 24 bp, Homo sapiens
  source 1..24: mol_type = other DNA
ctaagagagatcgttacctagcac

SEQ ID NO: 73 - DNA, 25 bp, Homo sapiens
  source 1..25: mol_type = other DNA
atcgttacctagcacccgttacctg

SEQ ID NO: 74 - DNA, 21 bp, Homo sapiens
  source 1..21: mol_type = other DNA
tagtggtttgccactccgacc

SEQ ID NO: 75 - DNA, 20 bp, Homo sapiens
  source 1..20: mol_type = other DNA
gccactccgaccaaaacacg

SEQ ID NO: 76 - DNA, 58 bp, Homo sapiens
  source 1..58: mol_type = genomic DNA
tcctaaccaagtgtctctttcccccagaatggctgcagtctgtccaggccaccatgat

SEQ ID NO: 77 - DNA, 25 bp, Homo sapiens
  source 1..25: mol_type = other DNA
aggattggttcacagagaaaggggg

SEQ ID NO: 78 - DNA, 25 bp, Homo sapiens
  source 1..25: mol_type = other DNA
ggattggttcacagagaaagggggt

SEQ ID NO: 79 - DNA, 25 bp, Homo sapiens
  source 1..25: mol_type = other DNA
gattggttcacagagaaagggggtc

SEQ ID NO: 80 - DNA, 25 bp, Homo sapiens
  source 1..25: mol_type = other DNA
attggttcacagagaaagggggtct

SEQ ID NO: 81 - DNA, 25 bp, Homo sapiens
  source 1..25: mol_type = other DNA
ttggttcacagagaaagggggtctt

SEQ ID NO: 82 - DNA, 25 bp, Homo sapiens
  source 1..25: mol_type = other DNA
tggttcacagagaaagggggtctta

SEQ ID NO: 83 - DNA, 25 bp, Homo sapiens
  source 1..25: mol_type = other DNA
ggttcacagagaaagggggtcttac

SEQ ID NO: 84 - DNA, 25 bp, Homo sapiens
  source 1..25: mol_type = other DNA
gttcacagagaaagggggtcttacc

SEQ ID NO: 85 - DNA, 25 bp, Homo sapiens
  source 1..25: mol_type = other DNA
ttcacagagaaagggggtcttaccg

SEQ ID NO: 86 - DNA, 25 bp, Homo sapiens
  source 1..25: mol_type = other DNA
tcacagagaaagggggtcttaccga

SEQ ID NO: 87 - DNA, 25 bp, Homo sapiens
  source 1..25: mol_type = other DNA
cacagagaaagggggtcttaccgac

SEQ ID NO: 88 - DNA, 25 bp, Homo sapiens
  source 1..25: mol_type = other DNA
acagagaaagggggtcttaccgacg

SEQ ID NO: 89 - DNA, 25 bp, Homo sapiens
  source 1..25: mol_type = other DNA
cagagaaagggggtcttaccgacgt

SEQ ID NO: 90 - DNA, 25 bp, Homo sapiens
  source 1..25: mol_type = other DNA
agagaaagggggtcttaccgacgtc

SEQ ID NO: 91 - DNA, 25 bp, Homo sapiens
  source 1..25: mol_type = other DNA
gagaaagggggtcttaccgacgtca

SEQ ID NO: 92 - DNA, 25 bp, Homo sapiens
  source 1..25: mol_type = other DNA
agaaagggggtcttaccgacgtcag

SEQ ID NO: 93 - DNA, 25 bp, Homo sapiens
  source 1..25: mol_type = other DNA
gaaagggggtcttaccgacgtcaga

SEQ ID NO: 94 - DNA, 25 bp, Homo sapiens
  source 1..25: mol_type = other DNA
aaagggggtcttaccgacgtcagac

SEQ ID NO: 95 - DNA, 25 bp, Homo sapiens
  source 1..25: mol_type = other DNA
aagggggtcttaccgacgtcagaca

SEQ ID NO: 96 - DNA, 25 bp, Homo sapiens
  source 1..25: mol_type = other DNA
agggggtcttaccgacgtcagacag

SEQ ID NO: 97 - DNA, 25 bp, Homo sapiens
  source 1..25: mol_type = other DNA
gggggtcttaccgacgtcagacagg

SEQ ID NO: 98 - DNA, 25 bp, Homo sapiens
  source 1..25: mol_type = other DNA
ggggtcttaccgacgtcagacaggt

SEQ ID NO: 99 - DNA, 25 bp, Homo sapiens
  source 1..25: mol_type = other DNA
gggtcttaccgacgtcagacaggtc

SEQ ID NO: 100 - DNA, 25 bp, Homo sapiens
  source 1..25: mol_type = other DNA
ggtcttaccgacgtcagacaggtcc

SEQ ID NO: 101 - DNA, 25 bp, Homo sapiens
  source 1..25: mol_type = other DNA
gtcttaccgacgtcagacaggtccg

SEQ ID NO: 102 - DNA, 25 bp, Homo sapiens
  source 1..25: mol_type = other DNA
tcttaccgacgtcagacaggtccgg

SEQ ID NO: 103 - DNA, 25 bp, Homo sapiens
  source 1..25: mol_type = other DNA
cttaccgacgtcagacaggtccggt

SEQ ID NO: 104 - DNA, 25 bp, Homo sapiens
  source 1..25: mol_type = other DNA
ttaccgacgtcagacaggtccggtg

SEQ ID NO: 105 - DNA, 25 bp, Homo sapiens
  source 1..25: mol_type = other DNA
taccgacgtcagacaggtccggtgg

SEQ ID NO: 106 - DNA, 25 bp, Homo sapiens
  source 1..25: mol_type = other DNA
accgacgtcagacaggtccggtggt

SEQ ID NO: 107 - DNA, 25 bp, Homo sapiens
  source 1..25: mol_type = other DNA
ccgacgtcagacaggtccggtggta

SEQ ID NO: 108 - DNA, 25 bp, Homo sapiens
  source 1..25: mol_type = other DNA
cgacgtcagacaggtccggtggtac

SEQ ID NO: 109 - DNA, 25 bp, Homo sapiens
  source 1..25: mol_type = other DNA
gacgtcagacaggtccggtggtact

SEQ ID NO: 110 - DNA, 25 bp, Homo sapiens
  source 1..25: mol_type = other DNA
acgtcagacaggtccggtggtacta

SEQ ID NO: 111 - DNA, 58 bp, Homo sapiens
  source 1..58: mol_type = genomic DNA
tacatcactggaatcttccaaattcttgctggtaagttgtggatggtaaagtccatgt

SEQ ID NO: 112 - DNA, 25 bp, Homo sapiens
  source 1..25: mol_type = other DNA
atgtagtgaccttagaaggtttaag

SEQ ID NO: 113 - DNA, 25 bp, Homo sapiens
  source 1..25: mol_type = other DNA
tgtagtgaccttagaaggtttaaga

SEQ ID NO: 114 - DNA, 25 bp, Homo sapiens
  source 1..25: mol_type = other DNA
gtagtgaccttagaaggtttaagaa

SEQ ID NO: 115 - DNA, 25 bp, Homo sapiens
  source 1..25: mol_type = other DNA
tagtgaccttagaaggtttaagaac

SEQ ID NO: 116 - DNA, 25 bp, Homo sapiens
  source 1..25: mol_type = other DNA
agtgaccttagaaggtttaagaacg

SEQ ID NO: 117 - DNA, 25 bp, Homo sapiens
  source 1..25: mol_type = other DNA
gtgaccttagaaggtttaagaacga

SEQ ID NO: 118 - DNA, 25 bp, Homo sapiens
  source 1..25: mol_type = other DNA
tgaccttagaaggtttaagaacgac

SEQ ID NO: 119 - DNA, 25 bp, Homo sapiens
  source 1..25: mol_type = other DNA
gaccttagaaggtttaagaacgacc

SEQ ID NO: 120 - DNA, 25 bp, Homo sapiens
  source 1..25: mol_type = other DNA
accttagaaggtttaagaacgacca

SEQ ID NO: 121 - DNA, 25 bp, Homo sapiens
  source 1..25: mol_type = other DNA
ccttagaaggtttaagaacgaccat

SEQ ID NO: 122 - DNA, 25 bp, Homo sapiens
  source 1..25: mol_type = other DNA
cttagaaggtttaagaacgaccatt

SEQ ID NO: 123 - DNA, 25 bp, Homo sapiens
  source 1..25: mol_type = other DNA
ttagaaggtttaagaacgaccattc

SEQ ID NO: 124 - DNA, 25 bp, Homo sapiens
  source 1..25: mol_type = other DNA
tagaaggtttaagaacgaccattca

SEQ ID NO: 125 - DNA, 25 bp, Homo sapiens
  source 1..25: mol_type = other DNA
agaaggtttaagaacgaccattcaa

SEQ ID NO: 126 - DNA, 25 bp, Homo sapiens
  source 1..25: mol_type = other DNA
gaaggtttaagaacgaccattcaac

SEQ ID NO: 127 - DNA, 25 bp, Homo sapiens
  source 1..25: mol_type = other DNA
aaggtttaagaacgaccattcaaca

SEQ ID NO: 128 - DNA, 25 bp, Homo sapiens
  source 1..25: mol_type = other DNA
aggtttaagaacgaccattcaacac

SEQ ID NO: 129 - DNA, 25 bp, Homo sapiens
  source 1..25: mol_type = other DNA
ggtttaagaacgaccattcaacacc

SEQ ID NO: 130 - DNA, 25 bp, Homo sapiens
  source 1..25: mol_type = other DNA
gtttaagaacgaccattcaacacct

SEQ ID NO: 131 - DNA, 25 bp, Homo sapiens
  source 1..25: mol_type = other DNA
tttaagaacgaccattcaacaccta

SEQ ID NO: 132 - DNA, 25 bp, Homo sapiens
  source 1..25: mol_type = other DNA
ttaagaacgaccattcaacacctac

SEQ ID NO: 133 - DNA, 25 bp, Homo sapiens
  source 1..25: mol_type = other DNA
taagaacgaccattcaacacctacc

SEQ ID NO: 134 - DNA, 25 bp, Homo sapiens
  source 1..25: mol_type = other DNA
aagaacgaccattcaacacctacca

SEQ ID NO: 135 - DNA, 25 bp, Homo sapiens
  source 1..25: mol_type = other DNA
agaacgaccattcaacacctaccat

SEQ ID NO: 136 - DNA, 25 bp, Homo sapiens
  source 1..25: mol_type = other DNA
gaacgaccattcaacacctaccatt

SEQ ID NO: 137 - DNA, 25 bp, Homo sapiens
  source 1..25: mol_type = other DNA
aacgaccattcaacacctaccattt

SEQ ID NO: 138 - DNA, 25 bp, Homo sapiens
  source 1..25: mol_type = other DNA
acgaccattcaacacctaccatttc

SEQ ID NO: 139 - DNA, 25 bp, Homo sapiens
  source 1..25: mol_type = other DNA
cgaccattcaacacctaccatttca

SEQ ID NO: 140 - DNA, 25 bp, Homo sapiens
  source 1..25: mol_type = other DNA
gaccattcaacacctaccatttcag

SEQ ID NO: 141 - DNA, 25 bp, Homo sapiens
  source 1..25: mol_type = other DNA
accattcaacacctaccatttcagg

SEQ ID NO: 142 - DNA, 25 bp, Homo sapiens
  source 1..25: mol_type = other DNA
ccattcaacacctaccatttcaggt

SEQ ID NO: 143 - DNA, 25 bp, Homo sapiens
  source 1..25: mol_type = other DNA
cattcaacacctaccatttcaggta

SEQ ID NO: 144 - DNA, 25 bp, Homo sapiens
  source 1..25: mol_type = other DNA
attcaacacctaccatttcaggtac

SEQ ID NO: 145 - DNA, 25 bp, Homo sapiens
  source 1..25: mol_type = other DNA
ttcaacacctaccatttcaggtaca

SEQ ID NO: 146 - DNA, 21 bp, Homo sapiens
  source 1..21: mol_type = other DNA
cacagagaaagggggtcttac

SEQ ID NO: 147 - DNA, 20 bp, Homo sapiens
  source 1..20: mol_type = other DNA
aaagggggtcttaccgacgt

SEQ ID NO: 148 - DNA, 20 bp, Homo sapiens
  source 1..20: mol_type = other DNA
ggtcttaccgacgtcagaca

SEQ ID NO: 149 - DNA, 21 bp, Homo sapiens
  source 1..21: mol_type = other DNA
gaacgaccattcaacacctac

SEQ ID NO: 150 - DNA, 20 bp, Homo sapiens
  source 1..20: mol_type = other DNA
cgaccattcaacacctacca

SEQ ID NO: 151 - DNA, 20 bp, Homo sapiens
  source 1..20: mol_type = other DNA
cattcaacacctaccatttc

SEQ ID NO: 152 - DNA, 35 bp, Homo sapiens
  source 1..35: mol_type = genomic DNA
tctgattctctctagcaatggatcgtgggcaatgg

SEQ ID NO: 153 - DNA, 30 bp, Homo sapiens
  source 1..30: mol_type = genomic DNA
catcatcaccaaacggtgaggctggttttg

SEQ ID NO: 154 - DNA, 13 bp, Homo sapiens
  source 1..13: mol_type = genomic DNA
ctagcaatggatc

SEQ ID NO: 155 - DNA, 11 bp, Homo sapiens
  source 1..11: mol_type = genomic DNA
cggtgaggctg

SEQ ID NO: 156 - DNA, 24 bp, Homo sapiens
  source 1..24: mol_type = other DNA
gatcgttacctaggccactccgac

SEQ ID NO: 157 - DNA, 13 bp, Homo sapiens
  source 1..13: mol_type = genomic DNA
gcaatggatcgtg

SEQ ID NO: 158 - DNA, 10 bp, Homo sapiens
  source 1..10: mol_type = genomic DNA
caccaaacgg

SEQ ID NO: 159 - DNA, 23 bp, Homo sapiens
  source 1..23: mol_type = other DNA
cgttacctagcacgtggtttgcc

SEQ ID NO: 160 - DNA, 13 bp, Homo sapiens
  source 1..13: mol_type = genomic DNA
ctctctagcaatg

SEQ ID NO: 161 - DNA, 11 bp, Homo sapiens
  source 1..11: mol_type = genomic DNA
caaacggtgag

SEQ ID NO: 162 - DNA, 24 bp, Homo sapiens
  source 1..24: mol_type = other DNA
gagagatcgttacgtttgccactc

SEQ ID NO: 163 - DNA, 58 bp, Homo sapiens
  source 1..58: mol_type = other DNA
gcctcacgcccagcgctctcctcgcaggcagaaactccgctgagcagaacttgccgcc

SEQ ID NO: 164 - DNA, 25 bp, Homo sapiens
  source 1..25: mol_type = other DNA
cggagtgcgggtcgcgagaggagcg

SEQ ID NO: 165 - DNA, 25 bp, Homo sapiens
  source 1..25: mol_type = other DNA
ggagtgcgggtcgcgagaggagcgt

SEQ ID NO: 166 - DNA, 25 bp, Homo sapiens
  source 1..25: mol_type = other DNA
gagtgcgggtcgcgagaggagcgtc

SEQ ID NO: 167 - DNA, 25 bp, Homo sapiens
  source 1..25: mol_type = other DNA
agtgcgggtcgcgagaggagcgtcc

SEQ ID NO: 168 - DNA, 25 bp, Homo sapiens
  source 1..25: mol_type = other DNA
gtgcgggtcgcgagaggagcgtccg

SEQ ID NO: 169 - DNA, 25 bp, Homo sapiens
  source 1..25: mol_type = other DNA
tgcgggtcgcgagaggagcgtccgt

SEQ ID NO: 170 - DNA, 25 bp, Homo sapiens
  source 1..25: mol_type = other DNA
gcgggtcgcgagaggagcgtccgtc

SEQ ID NO: 171 - DNA, 25 bp, Homo sapiens
  source 1..25: mol_type = other DNA
cgggtcgcgagaggagcgtccgtct

SEQ ID NO: 172 - DNA, 25 bp, Homo sapiens
  source 1..25: mol_type = other DNA
gggtcgcgagaggagcgtccgtctt

SEQ ID NO: 173 - DNA, 25 bp, Homo sapiens
  source 1..25: mol_type = other DNA
ggtcgcgagaggagcgtccgtcttt

SEQ ID NO: 174 - DNA, 25 bp, Homo sapiens
  source 1..25: mol_type = other DNA
gtcgcgagaggagcgtccgtctttg

SEQ ID NO: 175 - DNA, 25 bp, Homo sapiens
  source 1..25: mol_type = other DNA
tcgcgagaggagcgtccgtctttga

SEQ ID NO: 176 - DNA, 25 bp, Homo sapiens
  source 1..25: mol_type = other DNA
cgcgagaggagcgtccgtctttgag

SEQ ID NO: 177 - DNA, 25 bp, Homo sapiens
  source 1..25: mol_type = other DNA
gcgagaggagcgtccgtctttgagg

SEQ ID NO: 178 - DNA, 25 bp, Homo sapiens
  source 1..25: mol_type = other DNA
cgagaggagcgtccgtctttgaggc

SEQ ID NO: 179 - DNA, 25 bp, Homo sapiens
  source 1..25: mol_type = other DNA
gagaggagcgtccgtctttgaggcg

SEQ ID NO: 180 - DNA, 25 bp, Homo sapiens
  source 1..25: mol_type = other DNA
agaggagcgtccgtctttgaggcga

SEQ ID NO: 181 - DNA, 25 bp, Homo sapiens
  source 1..25: mol_type = other DNA
gaggagcgtccgtctttgaggcgac

SEQ ID NO: 182 - DNA, 25 bp, Homo sapiens
  source 1..25: mol_type = other DNA
aggagcgtccgtctttgaggcgact

SEQ ID NO: 183 - DNA, 25 bp, Homo sapiens
  source 1..25: mol_type = other DNA
ggagcgtccgtctttgaggcgactc

SEQ ID NO: 184 - DNA, 25 bp, Homo sapiens
  source 1..25: mol_type = other DNA
gagcgtccgtctttgaggcgactcg

SEQ ID NO: 185 - DNA, 25 bp, Homo sapiens
  source 1..25: mol_type = other DNA
agcgtccgtctttgaggcgactcgt

SEQ ID NO: 186 - DNA, 25 bp, Homo sapiens
  source 1..25: mol_type = other DNA
gcgtccgtctttgaggcgactcgtc

SEQ ID NO: 187 - DNA, 25 bp, Homo sapiens
  source 1..25: mol_type = other DNA
cgtccgtctttgaggcgactcgtct

SEQ ID NO: 188 - DNA, 25 bp, Homo sapiens
  source 1..25: mol_type = other DNA
gtccgtctttgaggcgactcgtctt

SEQ ID NO: 189 - DNA, 25 bp, Homo sapiens
  source 1..25: mol_type = other DNA
tccgtctttgaggcgactcgtcttg

SEQ ID NO: 190 - DNA, 25 bp, Homo sapiens
  source 1..25: mol_type = other DNA
ccgtctttgaggcgactcgtcttga

SEQ ID NO: 191 - DNA, 25 bp, Homo sapiens
  source 1..25: mol_type = other DNA
cgtctttgaggcgactcgtcttgaa

SEQ ID NO: 192 - DNA, 25 bp, Homo sapiens
  source 1..25: mol_type = other DNA
gtctttgaggcgactcgtcttgaac

SEQ ID NO: 193 - DNA, 25 bp, Homo sapiens
  source 1..25: mol_type = other DNA
tctttgaggcgactcgtcttgaacg

SEQ ID NO: 194 - DNA, 25 bp, Homo sapiens
  source 1..25: mol_type = other DNA
ctttgaggcgactcgtcttgaacgg

SEQ ID NO: 195 - DNA, 25 bp, Homo sapiens
  source 1..25: mol_type = other DNA
tttgaggcgactcgtcttgaacggc

SEQ ID NO: 196 - DNA, 25 bp, Homo sapiens
  source 1..25: mol_type = other DNA
ttgaggcgactcgtcttgaacggcg

SEQ ID NO: 197 - DNA, 25 bp, Homo sapiens
  source 1..25: mol_type = other DNA
tgaggcgactcgtcttgaacggcgg

SEQ ID NO: 198 - DNA, 58 bp, Homo sapiens
  source 1..58: mol_type = genomic DNA
ggtgctgctgttcgtctccacgatcgtcagcgtgagtgcctggcggggaggctccctg

SEQ ID NO: 199 - DNA, 25 bp, Homo sapiens
  source 1..25: mol_type = other DNA
ccacgacgacaagcagaggtgctag

SEQ ID NO: 200 - DNA, 25 bp, Homo sapiens
  source 1..25: mol_type = other DNA
cacgacgacaagcagaggtgctagc

SEQ ID NO: 201 - DNA, 25 bp, Homo sapiens
  source 1..25: mol_type = other DNA
acgacgacaagcagaggtgctagca

SEQ ID NO: 202 - DNA, 25 bp, Homo sapiens
  source 1..25: mol_type = other DNA
cgacgacaagcagaggtgctagcag

SEQ ID NO: 203 - DNA, 25 bp, Homo sapiens
  source 1..25: mol_type = other DNA
gacgacaagcagaggtgctagcagt

SEQ ID NO: 204 - DNA, 25 bp, Homo sapiens
  source 1..25: mol_type = other DNA
acgacaagcagaggtgctagcagtc

SEQ ID NO: 205 - DNA, 25 bp, Homo sapiens
  source 1..25: mol_type = other DNA
cgacaagcagaggtgctagcagtcg

SEQ ID NO: 206 - DNA, 25 bp, Homo sapiens
  source 1..25: mol_type = other DNA
gacaagcagaggtgctagcagtcgc

SEQ ID NO: 207 - DNA, 25 bp, Homo sapiens
  source 1..25: mol_type = other DNA
acaagcagaggtgctagcagtcgca

SEQ ID NO: 208 - DNA, 25 bp, Homo sapiens
  source 1..25: mol_type = other DNA
caagcagaggtgctagcagtcgcac

SEQ ID NO: 209 - DNA, 25 bp, Homo sapiens
  source 1..25: mol_type = other DNA
aagcagaggtgctagcagtcgcact

SEQ ID NO: 210 - DNA, 25 bp, Homo sapiens
  source 1..25: mol_type = other DNA
agcagaggtgctagcagtcgcactc

SEQ ID NO: 211 - DNA, 25 bp, Homo sapiens
  source 1..25: mol_type = other DNA
gcagaggtgctagcagtcgcactca

SEQ ID NO: 212 - DNA, 25 bp, Homo sapiens
  source 1..25: mol_type = other DNA
cagaggtgctagcagtcgcactcac

SEQ ID NO: 213 - DNA, 25 bp, Homo sapiens
  source 1..25: mol_type = other DNA
agaggtgctagcagtcgcactcacg

SEQ ID NO: 214 - DNA, 25 bp, Homo sapiens
  source 1..25: mol_type = other DNA
gaggtgctagcagtcgcactcacgg

SEQ ID NO: 215 - DNA, 25 bp, Homo sapiens
  source 1..25: mol_type = other DNA
aggtgctagcagtcgcactcacgga

SEQ ID NO: 216 - DNA, 25 bp, Homo sapiens
  source 1..25: mol_type = other DNA
ggtgctagcagtcgcactcacggac

SEQ ID NO: 217 - DNA, 25 bp, Homo sapiens
  source 1..25: mol_type = other DNA
gtgctagcagtcgcactcacggacc

SEQ ID NO: 218 - DNA, 25 bp, Homo sapiens
  source 1..25: mol_type = other DNA
tgctagcagtcgcactcacggaccg

SEQ ID NO: 219 - DNA, 25 bp, Homo sapiens
  source 1..25: mol_type = other DNA
gctagcagtcgcactcacggaccgc

SEQ ID NO: 220 - DNA, 25 bp, Homo sapiens
  source 1..25: mol_type = other DNA
ctagcagtcgcactcacggaccgcc

SEQ ID NO: 221 - DNA, 25 bp, Homo sapiens
  source 1..25: mol_type = other DNA
tagcagtcgcactcacggaccgccc

SEQ ID NO: 222 - DNA, 25 bp, Homo sapiens
  source 1..25: mol_type = other DNA
agcagtcgcactcacggaccgcccc

SEQ ID NO: 223 - DNA, 25 bp, Homo sapiens
  source 1..25: mol_type = other DNA
gcagtcgcactcacggaccgcccct

SEQ ID NO: 224 - DNA, 25 bp, Homo sapiens
  source 1..25: mol_type = other DNA
cagtcgcactcacggaccgcccctc

SEQ ID NO: 225 - DNA, 25 bp, Homo sapiens
  source 1..25: mol_type = other DNA
agtcgcactcacggaccgcccctcc

SEQ ID NO: 226 - DNA, 25 bp, Homo sapiens
  source 1..25: mol_type = other DNA
gtcgcactcacggaccgcccctccg

SEQ ID NO: 227 - DNA, 25 bp, Homo sapiens
  source 1..25: mol_type = other DNA
tcgcactcacggaccgcccctccga

SEQ ID NO: 228 - DNA, 25 bp, Homo sapiens
  source 1..25: mol_type = other DNA
cgcactcacggaccgcccctccgag

SEQ ID NO: 229 - DNA, 25 bp, Homo sapiens
  source 1..25: mol_type = other DNA
gcactcacggaccgcccctccgagg

SEQ ID NO: 230 - DNA, 25 bp, Homo sapiens
  source 1..25: mol_type = other DNA
cactcacggaccgcccctccgaggg

SEQ ID NO: 231 - DNA, 25 bp, Homo sapiens
  source 1..25: mol_type = other DNA
actcacggaccgcccctccgaggga

SEQ ID NO: 232 - DNA, 25 bp, Homo sapiens
  source 1..25: mol_type = other DNA
ctcacggaccgcccctccgagggac

SEQ ID NO: 233 - DNA, 13 bp, Homo sapiens
  source 1..13: mol_type = genomic DNA
gcaatggatcgtg

SEQ ID NO: 234 - DNA, 11 bp, Homo sapiens
  source 1..11: mol_type = genomic DNA
gtgaggctggt

SEQ ID NO: 235 - DNA, 24 bp, Homo sapiens
  source 1..24: mol_type = other DNA
cgttacctagcaccactccgacca

SEQ ID NO: 236 - DNA, 13 bp, Homo sapiens
  source 1..13: mol_type = genomic DNA
gattctctctagc

SEQ ID NO: 237 - DNA, 11 bp, Homo sapiens
  source 1..11: mol_type = genomic DNA
tcaccaaacgg

SEQ ID NO: 238 - DNA, 24 bp, Homo sapiens
  source 1..24: mol_type = other DNA
ctaagagagatcgagtggtttgcc

SEQ ID NO: 239 - DNA, 21 bp, Homo sapiens
  source 1..21: mol_type = other DNA
gaagaggtgctacagttctgc